(19) United States
(12) Patent Application Publication
(10) Pub. No.: US 2006/0024794 A1
(43) Pub. Date: Feb. 2, 2006

(54) Novel methods for production of di-chain botulinum toxin

(76) Inventors: Shengwen Li, Irvine, CA (US); Kei Roger Aoki, Coto de Caza, CA (US)

Correspondence Address: ALLERGAN, INC., LEGAL DEPARTMENT, 2525 DUPONT DRIVE, T2-7H, IRVINE, CA, 92612-1599, US

(21) Appl. No.: 10/903,375

(22) Filed: Jul. 30, 2004

(51) Int. Cl. C07H 21/04; C12P 21/04; C12N 15/74; C07K 14/33

(52) U.S. Cl. 435/69.7; 435/472; 435/252.3; 530/350; 536/23.7

(57) ABSTRACT

The present invention relates to methods of manufacturing a di-chain botulinum toxin, wherein the methods do not involve the process of producing a single chain botulinum toxin that is followed by nicking to form a di-chain botulinum toxin.

### FIELD OF THE INVENTION

This invention broadly relates to recombinant DNA technology. Particularly, the invention is directed to methods of manufacturing a di-chain botulinum toxin, wherein the methods do not involve the process of producing a single chain botulinum toxin which is followed by nicking to form a di-chain botulinum toxin.

### BACKGROUND OF THE INVENTION

Botulinum toxins have been used in clinical settings for the treatment of neuromuscular disorders characterized by hyperactive skeletal muscles. In 1989 a botulinum toxin serotype A complex has been approved by the U.S. Food and Drug Administration for the treatment of blepharospasm, strabismus and hemifacial spasm. Subsequently, a botulinum toxin serotype A was also approved by the FDA for the treatment of cervical dystonia and for the treatment of glabellar lines, and a botulinum toxin serotype B was approved for the treatment of cervical dystonia. Non-type A botulinum toxin serotypes apparently have a lower potency and/or a shorter duration of activity as compared to botulinum toxin serotype A. Clinical effects of peripheral intramuscular botulinum toxin serotype A are usually seen within one week of injection. The typical duration of symptomatic relief from a single intramuscular injection of botulinum toxin serotype A averages about three months, although significantly longer periods of therapeutic activity have been reported.

It has been reported that botulinum toxin serotype A has been used in clinical settings as follows:
(1) about 75-125 units of BOTOX® per intramuscular injection (multiple muscles) to treat cervical dystonia;
(2) 5-10 units of BOTOX® per intramuscular injection to treat glabellar lines (brow furrows) (5 units injected intramuscularly into the procerus muscle and 10 units injected intramuscularly into each corrugator supercilii muscle);
(3) about 30-80 units of BOTOX® to treat constipation by intrasphincter injection of the puborectalis muscle;
(4) about 1-5 units per muscle of intramuscularly injected BOTOX® to treat blepharospasm by injecting the lateral pre-tarsal orbicularis oculi muscle of the upper lid and the lateral pre-tarsal orbicularis oculi of the lower lid.
(5) to treat strabismus, extraocular muscles have been injected intramuscularly with between about 1-5 units of BOTOX®, the amount injected varying based upon both the size of the muscle to be injected and the extent of muscle paralysis desired (i.e. amount of diopter correction desired).
(6) to treat upper limb spasticity following stroke by intramuscular injections of BOTOX® into five different upper limb flexor muscles, as follows:
   (a) flexor digitorum profundus: 7.5 U to 30 U
   (b) flexor digitorum sublimus: 7.5 U to 30 U
   (c) flexor carpi ulnaris: 10 U to 40 U
   (d) flexor carpi radialis: 15 U to 60 U
   (e) biceps brachii: 50 U to 200 U.
      Each of the five indicated muscles has been injected at the same treatment session, so that the patient receives from 90 U to 360 U of upper limb flexor muscle BOTOX® by intramuscular injection at each treatment session.
(7) to treat migraine, pericranial injected (injected symmetrically into glabellar, frontalis and temporalis muscles) injection of 25 U of BOTOX® has showed significant benefit as a prophylactic treatment of migraine compared to vehicle as measured by decreased measures of migraine frequency, maximal severity, associated vomiting and acute medication use over the three month period following the 25 U injection.

Additionally, intramuscular botulinum toxin has been used in the treatment of tremor in patient's with Parkinson's disease, although it has been reported that results have not been impressive. Marjama-Jyons, J., et al., Tremor-Predominant Parkinson's Disease, Drugs & Aging 16(4); 273-278:2000.

It is known that botulinum toxin serotype A can have an efficacy for up to 12 months (European J. Neurology 6 (Supp 4): S111-S1150:1999), and in some circumstances for as long as 27 months. The Laryngoscope 109:1344-1346:1999. However, the usual duration of an intramuscular injection of Botox® is typically about 3 to 4 months.

The success of botulinum toxin serotype A to treat a variety of clinical conditions has led to interest in other botulinum toxin serotypes. Two commercially available botulinum serotype A preparations for use in humans are BOTOX® available from Allergan, Inc., of Irvine, Calif., and Dysport® available from Beaufour Ipsen, Porton Down, England. A Botulinum toxin serotype B preparation (MyoBloc®) is available from Elan Pharmaceuticals of San Francisco, Calif.

In addition to having pharmacologic actions at the peripheral location, botulinum toxins may also have inhibitory effects in the central nervous system. Work by Weigand et al, Nauny-Schmiedeberg's Arch. Pharmacol. 1976; 292, 161-165, and Habermann, Nauny-Schmiedeberg's Arch. Pharmacol. 1974; 281, 47-56 showed that botulinum toxin is able to ascend to the spinal area by retrograde transport. As such, a botulinum toxin injected at a peripheral location, for example intramuscularly, may be retrograde transported to the spinal cord.

A botulinum toxin has also been proposed for the treatment of rhinorrhea, hyperhydrosis and other disorders mediated by the autonomic nervous system (U.S. Pat. No. 5,766,605), tension headache, (U.S. Pat. No. 6,458,365), migraine headache (U.S. Pat. No. 5,714,468), post-operative pain and visceral pain (U.S. Pat. No. 6,464,986), pain treatment by intraspinal toxin administration (U.S. Pat. No. 6,113,915), Parkinson's disease and other diseases with a motor disorder component, by intracranial toxin administration (U.S. Pat. No. 6,306,403), hair growth and hair retention (U.S. Pat. No. 6,299,893), psoriasis and dermatitis (U.S. Pat. No. 5,670,484), injured muscles (U.S. Pat. No. 6,423,319, various cancers (U.S. Pat. No. 6,139,845), pancreatic disorders (U.S. Pat. No. 6,143,306), smooth muscle disorders (U.S. Pat. No. 5,437,291, including injection of a botulinum toxin into the upper and lower esophageal, pyloric and anal sphincters)), prostate disorders (U.S. Pat. No. 6,365,164), inflammation, arthritis and gout (U.S. Pat. No. 6,063,768), juvenile cerebral palsy (U.S. Pat. No. 6,395,277), inner ear disorders (U.S. Pat. No. 6,265,379), thyroid disorders (U.S. Pat. No. 6,358,513), parathyroid disorders (U.S. Pat. No. 6,328,977). Additionally, controlled release toxin implants are known (see e.g. U.S. Pat. Nos. 6,306,423 and 6,312,708).

Seven generally immunologically distinct botulinum neurotoxins have been characterized: botulinum neurotoxin serotypes (types) A, B, C₁, D, E, F and G. These serotypes are distinguished by neutralization with serotype-specific antibodies. The different serotypes of botulinum toxin vary in the animal species that they affect and in the severity and duration of the paralysis they evoke. For example, it has been determined that botulinum toxin serotype A is 500 times more potent, as measured by the rate of paralysis produced in the rat, than is botulinum toxin serotype B. Additionally, botulinum toxin serotype B has been determined to be non-toxic in primates at a dose of 480 U/kg which is about 12 times the primate LD₅₀ for botulinum toxin serotype A. Moyer E et al., Botulinum Toxin serotype B: Experimental and Clinical Experience, being chapter 6, pages 71-85 of “Therapy With Botulinum Toxin”, edited by Jankovic, J. et al. (1994), Marcel Dekker, Inc. Botulinum toxin apparently binds with high affinity to cholinergic motor neurons, is translocated into the neuron and blocks the release of acetylcholine.

Regardless of serotype, the molecular mechanism of toxin intoxication appears to be similar and to involve at least three steps or stages. In the first step of the process, the toxin binds to the presynaptic membrane of the target neuron through a specific interaction between the heavy chain, H chain, and a cell surface receptor; the receptor is thought to be different for each serotype of botulinum toxin and for tetanus toxin. The carboxyl end segment of the H chain, H_{C}, appears to be important for targeting of the toxin to the cell surface.

In the second step, the toxin crosses the plasma membrane of the poisoned cell. The toxin is first engulfed by the cell through receptor-mediated endocytosis, and an endosome containing the toxin is formed. The toxin then escapes the endosome into the cytoplasm of the cell. This step is thought to be mediated by the amino end segment of the H chain, H_{N}, which triggers a conformational change of the toxin in response to a pH of about 5.5 or lower. Endosomes are known to possess a proton pump which decreases intra-endosomal pH. The conformational shift exposes hydrophobic residues in the toxin, which permits the toxin to embed itself in the endosomal membrane. The toxin (or at a minimum the light chain) then translocates through the endosomal membrane into the cytoplasm.

The last step of the mechanism of botulinum toxin activity appears to involve reduction of the disulfide bond joining the heavy chain, H chain, and the light chain, L chain. The entire toxic activity of botulinum and tetanus toxins is contained in the L chain of the holotoxin; the L chain is a zinc (Zn++) endopeptidase which selectively cleaves proteins essential for recognition and docking of neurotransmitter-containing vesicles with the cytoplasmic surface of the plasma membrane, and fusion of the vesicles with the plasma membrane. Tetanus neurotoxin, botulinum toxin serotypes B, D, F, and G cause degradation of synaptobrevin (also called vesicle-associated membrane protein (VAMP)), a synaptosomal membrane protein. Most of the VAMP present at the cytoplasmic surface of the synaptic vesicle is removed as a result of any one of these cleavage events. Botulinum toxin serotype A and E cleave SNAP-25. Botulinum toxin serotype C₁ was originally thought to cleave syntaxin, but was found to cleave syntaxin and SNAP-25. Each of the botulinum toxins specifically cleaves a different bond, except botulinum toxin serotype B (and tetanus toxin) which cleave the same bond.

Although all the botulinum toxins serotypes apparently inhibit release of the neurotransmitter acetylcholine at the neuromuscular junction, they do so by affecting different neurosecretory proteins and/or cleaving these proteins at different sites. For example, botulinum serotypes A and E both cleave the 25 kiloDalton (kD) synaptosomal associated protein (SNAP-25), but they target different amino acid sequences within this protein. Botulinum toxin serotypes B, D, F and G act on vesicle-associated protein (VAMP, also called synaptobrevin), with each serotype cleaving the protein at a different site. Finally, botulinum toxin serotype C₁ has been shown to cleave both syntaxin and SNAP-25. These differences in mechanism of action may affect the relative potency and/or duration of action of the various botulinum toxin serotypes. Apparently, a substrate for a botulinum toxin can be found in a variety of different cell serotypes. See e.g. Biochem, J 1; 339 (pt 1):159-65:1999, and Mov Disord, 10(3):376:1995 (pancreatic islet B cells contains at least SNAP-25 and synaptobrevin).

The molecular weight of the botulinum toxin protein molecule, for all seven of the known botulinum toxin serotypes, is about 150 kD. Interestingly, the botulinum toxins are released by Clostridial bacterium as complexes comprising the 150 kD botulinum toxin protein molecule along with associated non-toxin proteins. Thus, the botulinum toxin serotype A complex can be produced by Clostridial bacterium as 900 kD, 500 kD and 300 kD forms. Botulinum toxin serotypes B and C₁ is apparently produced as only a 700 kD or 500 kD complex. Botulinum toxin serotype D is produced as both 300 kD and 500 kD complexes. Finally, botulinum toxin serotypes E and F are produced as only approximately 300 kD complexes. The complexes (i.e. molecular weight greater than about 150 kD) are believed to contain a non-toxin hemagglutinin protein and a non-toxin and non-toxic nonhemagglutinin protein. These two non-toxin proteins (which along with the botulinum toxin molecule comprise the relevant neurotoxin complex) may act to provide stability against denaturation to the botulinum toxin molecule and protection against digestive acids when toxin is ingested. Additionally, it is possible that the larger (greater than about 150 kD molecular weight) botulinum toxin complexes may result in a slower rate of diffusion of the botulinum toxin away from a site of intramuscular injection of a botulinum toxin complex.

In vitro studies have indicated that botulinum toxin inhibits potassium cation induced release of both acetylcholine and norepinephrine from primary cell cultures of brainstem tissue. Additionally, it has been reported that botulinum toxin inhibits the evoked release of both glycine and glutamate in primary cultures of spinal cord neurons and that in brain synaptosome preparations botulinum toxin inhibits the release of each of the neurotransmitters acetylcholine, dopamine, norepinephrine (Habermann E., et al., Tetanus Toxin and Botulinum A and C Neurotoxins Inhibit Noradrenaline Release From Cultured Mouse Brain, J Neurochem 51 (2);522-527:1988) CGRP, substance P and glutamate (Sanchez-Prieto, J., et al., Botulinum Toxin A Blocks Glutamate Exocytosis From Guinea Pig Cerebral Cortical Synaptosomes, Eur J. Biochem 165;675-681:1897. Thus, when adequate concentrations are used, stimulus-evoked release of most neurotransmitters is blocked by botulinum toxin. See e.g. Pearce, L. B., Pharmacologic Characterization of Botulinum Toxin For Basic Science and Medicine, Toxicon 35 (9);1373-1412 at 1393; Bigalke H., et al., Botulinum A Neurotoxin Inhibits Non-Cholinergic Synaptic Transmission in Mouse Spinal Cord Neurons in Culture, Brain Research 360;318-324:1985; Habermann E., Inhibition by Tetanus and Botulinum A Toxin of the release of [³H]Noradrenaline and [³H]GABA From Rat Brain Homogenate, Experientia 44;224-226: 1988, Bigalke H., et al., Tetanus Toxin and Botulinum A Toxin Inhibit Release and Uptake of Various Transmitters, as Studied with Particulate Preparations From Rat Brain and Spinal Cord, Naunyn-Schmiedeberg's Arch Pharmacol 316;244-251:1981, and; Jankovic J. et al., Therapy With Botulinum Toxin, Marcel Dekker, Inc., (1994), page 5.

A commercially available botulinum toxin containing pharmaceutical composition is sold under the trademark BOTOX® (available from Allergan, Inc., of Irvine, Calif.). BOTOX® consists of a purified botulinum toxin serotype A complex, albumin and sodium chloride packaged in sterile, vacuum-dried form. The botulinum toxin serotype A is made from a culture of the Hall strain of Clostridium botulinum grown in a medium containing N-Z amine and yeast extract. The botulinum toxin serotype A complex is purified from the culture solution by a series of acid precipitations to a crystalline complex consisting of the active high molecular weight toxin protein and an associated hemagglutinin protein. The crystalline complex is re-dissolved in a solution containing saline and albumin and sterile filtered (0.2 microns) prior to vacuum-drying. The vacuum-dried product is stored in a freezer at or below −5° C. BOTOX® can be reconstituted with sterile, non-preserved saline prior to intramuscular injection. Each vial of BOTOX® contains about 100 units (U) of Clostridium botulinum toxin serotype A purified neurotoxin complex, 0.5 milligrams of human serum albumin and 0.9 milligrams of sodium chloride in a sterile, vacuum-dried form without a preservative.

To reconstitute vacuum-dried BOTOX®, sterile normal saline without a preservative; (0.9% Sodium Chloride Injection) is used by drawing up the proper amount of diluent in the appropriate size syringe. Since BOTOX® may be denatured by bubbling or similar violent agitation, the diluent is gently injected into the vial. For sterility reasons BOTOX® is preferably administered within four hours after the vial is removed from the freezer and reconstituted. During these four hours, reconstituted BOTOX® can be stored in a refrigerator at about 2° C. to about 8° C. Reconstituted, refrigerated BOTOX® has been reported to retain its potency for at least about two weeks. Neurology, 48:249-53:1997.

Generally, commercial botulinum toxins are produced by establishing and growing cultures of Clostridium botulinum, E. coli cells or recombinantly engineered yeast cells in a fermenter and then harvesting and purifying the fermented mixture in accordance with known procedures. All the botulinum toxin serotypes are initially synthesized as inactive single chain proteins. To be converted into their active forms, the single chain botulinum toxins are subsequently nicked by proteases, e.g. trypsin.

Although the use of trypsin is an effective way to make di-chain botulinum toxins, the use of trypsin poses several difficulties. For example, the trypsin nicking digestion is hard to control. If over-digested, the toxin loses its therapeutic effect due to the degradation. If under-digested, the toxin is partially activated, which result in low efficacy. Moreover, in order for botulinum toxin to be used as a protein drug, the FDA requires that the botulinum toxin is free from trypsin, which may introduce immunogenic problems in patients.

Thus, there remains a need to have improved methods for manufacturing a di-chain botulinum toxin, which do not require the use of a protease (i.e. trypsin) to nick the single chain chain botulinum toxin.

### SUMMARY OF THE INVENTION

The present invention meets this need and provides for more effective methods of manufacturing di-chain botulinum toxins. In accordance with the present invention, methods of manufacturing a di-chain botulinum toxin comprising expressing a botulinum toxin light chain and a botulinum toxin heavy chain separately in a same cell are provided.

In some embodiments, one or more vectors are used for expressing the botulinum toxin light chain and the botulinum heavy chain in the cell. For example, a single vector may be used for expressing the botulinum toxin light chain and the botulinum toxin heavy chain. In another example, two vectors may be used, wherein the first vector is employed for expressing the botulinum toxin light chain and a second vector is employed for expressing the botulinum toxin heavy chain.

In some embodiments, the vectors used in accordance with the present invention are viral-based expression vector, plasmid-based expression vector, yeast expression vector, bacterial expression vector, a plant expression vector, amphibian expression vector, mammalian expression vector and/or recombinant baculovirus vector.

In some embodiments, cells used in accordance with the present invention include prokaryotic cells and eukaryotic cells. Non-limiting examples of prokaryotic cell are Escherichia coli cells, Clostridium botulinum cell, Clostridium tetani cells, Clostridium beratti cells, Clostridium butyricum cells, or Clostridium perfringens cells.

In some embodiments, a light chain and a heavy chain are separately expressed in an Escherichia coli cell, wherein the light chain and heavy chain form a disulfide bridge with each other after they are separately expressed in the Escherichia coli cell.

Non-limiting examples of eukaryotic cells are insect cells, yeast cells, amphibian cells, mammalian cell, plant cells. Non-limiting examples of insect cells are Spodoptera frugiperda cells, Aedes albopictus cells, Trichoplusia ni cells, Estigmene acrea cells, Bombyx mori cells and Drosophila melanogaster cells. Non-limiting examples of yeast cells are Saccharomyces cerevisiae cells, Schizosaccharomyces pombe cells, Pichia pastoris cells, Hansenula polymorpha cells, Kluyveromyces lactis cells and Yarrowia lipolytica cells.

In some embodiments, a botulinum toxin light chain is a light chain of Clostridium botulinum toxin serotypes A, B, C1, D, E, F or G. In some embodiments, a botulinum toxin heavy chain is a heavy chain of Clostridium botulinum toxin serotypes A, B, C1, D, E, F or G.

In some embodiments, one or more accessory proteins are co-expressed with the light chain and heavy chain in the cell, whereby the accessory protein facilitates the disulfide bridge formation between the light chain and the heavy chain. Non-limiting examples of accessory proteins include NTNH, HA70, HA34, HA17, GroES, GroEL, disulfide isomerase or heat shock protein.

In accordance with the present invention, a vector comprising a baculovirus promoter operably linked to a light chain of a botulinum toxin or a heavy chain of a botulinum toxin is provided. In some embodiments, the promoter may be a polyhedrin or polypeptide 10 (p10) promoter.

In accordance with the present invention, a host cell comprising a vector which comprises a baculovirus promoter operably linked to a light chain of a botulinum toxin or a heavy chain of a botulinum toxin is provided. In some embodiments, the host cell may be a prokaryotic cell or a eukaryotic cell. In some embodiments, the host cell is an insect cell, for example an Sf9 cell, an Sf21 cell, or a BTI-Tn-5B1-4 cell.

In accordance with the present invention, a di-chain botulinum toxin is provided, wherein said toxin is made by expressing a botulinum toxin light chain and a botulinum toxin heavy chain separately in a same cell, whereby the light chain forms a disulfide bridge with the heavy chain to form a di-chain botulinum toxin.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

Definitions

The term “promoter” means a DNA sequence at the 5′-end of a structural gene that is capable of initiating transcription. For example, one promoter of the present invention is the promoter for the Baculovirus nonessential gene, polyhedrin. Other Baculovirus promoters include the p10 promoter and those described by Vialard et al. J. Virol. 64:37-50 (1990); and Vlak et al. Virology 179:312-320 (1990). In order for the promoter to initiate transcription, the coding sequence for a desired protein must be inserted “downstream,” “3″” or “behind” the promoter.

The term “operably linked” means two sequences of a nucleic acid molecule which are linked to each other in a manner which either permits both sequences to be transcribed onto the same RNA transcript, or permits an RNA transcript, begun in one sequence, to be extended into the second sequence. Thus, two sequences, such as a promoter and any other “second” sequence of DNA (or RNA) are operably linked if transcription commencing in the promoter sequence will produce an RNA (or cDNA) transcript of the operably linked second sequence. In order to be “operably linked” it is not necessary that two sequences be immediately adjacent to one another.

The term “vector” means a nucleic acid sequence used as a vehicle for cloning or expressing a fragment of a foreign nucleic acid sequence. And a “vector operably harboring a nucleic acid sequence” means a vector comprising the nucleic acid sequence and is capable of expressing such nucleic acid sequence.

The term “transforming” means the act of causing a cell to contain a nucleic acid molecule or sequence not originally part of that cell. This is the process by which DNA is introduced into a cell. Methods of transformation are known in the art. See e.g., Maniatis et al., Molecular Cloning: A Laboratory Manual, (Cold Spring Harbor Laboratory, Publisher, N.Y. (2d ed. 1989).

The term “transfecting” is intended the introduction of viral DNA or RNA, e.g., a vector, into any cell.

The term “host” or “host cell” means the cell in which a vector is transformed. Once the foreign DNA is incorporated into the host cell, the host cell may express the foreign DNA. For example, the “host cell” of the present invention includes Sf9, a clonal isolate of the IPLB-Sf21-AE line established from Spodoptera frugiperda, commonly known as the fall army worm.

The term “baculovirus” means a member of the Baculoviridae family of viruses with covalently closed double-stranded DNA genome and which are pathogenic for invertebrates, primarily insects of the order Lepidoptera.

The term “botulinum toxin” (“BoNT”) means active or inactive botulinum toxin, unless it is specifically designated as inactive botulinum toxin (“iBoNT) or active BoNT.

The term “single chain botulinum toxin” means a BoNT having a light chain and a heavy chain being within a single peptide.

The term “di-chain botulinum toxin” means a BoNT having two peptides, i.e., the light chain and the heavy chain, being linked by a disulfide bridge.

The term “heavy chain” (HC) means the heavy chain of a BoNT. It has a molecular weight of about 100 kDa and can be referred to herein as heavy chain or as H.

The term “light chain” (LC) means the light chain of a BoNT. It has a molecular weight of about 50 kDa, and can be referred to as light chain, LC or as the proteolytic domain (amino acid sequence) of a BoNT. The light chain is believed to be effective as an inhibitor of exocytosis, including as an inhibitor of neurotransmitter (i.e. acetylcholine) release when the light chain is present in the cytoplasm of a target cell.

The term “active botulinum toxin” means a BoNT that is capable of substantially inhibiting release of neurotransmitters from nerve terminals or cells.

The term “inactive botulinum toxin” (“iBoNT”) means a BoNT that is not toxic to a cell. For example, an iBoNT has minimal or no ability to interfere with the release of neurotransmitters from a cell or nerve endings. In some embodiments, the iBoNT has no neurotoxic effect (e.g., no ability to inhibit release of neurotransmitter or no ability to cleavage substrates). In some embodiments, the iBoNT has less than about 50% of the neurotoxic effect of an identical BoNT that is active. For example, an iBoNT/A has less than about 50% of the neurotoxic effect of an identical BoNT/A that is active. In some embodiments, the iBoNT has less than about 25% of the neurotoxic effect of an identical BoNT that is active. In some embodiments, the iBoNT has less than about 10% of the neurotoxic effect of an identical BoNT that is active. In some embodiments, the iBoNT has less than about 5% of the neurotoxic effect of an identical BoNT that is active. Inactive botulinum toxins are well known to those skilled in the art. For example, see U.S. Pat. No. 6,051,239 to Simpson et al. In some embodiments, the iBoNT comprises a heavy chain and a light chain, wherein the light chain is mutated as to have minimal or no ability to directly interfere with the release of neurotransmitters from a cell or a nerve ending. However, the iBoNT may have the ability to compete with an active BoNT. In some embodiments, the heavy chain is modified as to reduce antigenicity. In some embodiments, iBoNT is a single chain peptide.

The term “mammal” as used herein includes, for example, humans, rats, rabbits, mice and dogs.

The term “local administration” means direct administration by a non-systemic route at or in the vicinity of the site of an affliction, disorder or perceived pain.

### BRIEF DESCRIPTION OF THE DRAWINGS

FIG. 1 shows a PCR amplified BoNT/A-LC. Lane M is the DNA 1 Kb Ladder; lane 1 is the Wild type LCA; lane 2 is the Mutant LCA; and lane 3 is the Negative Control.

FIGS. 2A and 2B show the selection and confirmation of the positive clones by PCR Screening and restriction enzymes digestion, respectively.

FIG. 3 shows the Glucuronidase enzymatic activity assay of rLC/A (wt, mt), which indicated the generation of the recombinant baculoviruses.

FIG. 4 shows the expression of rLC/A revealed by SDS-PAGE and Coomassie blue staining. Lane M is the Blue Plus2 marker; lane 1 is the pBAC-1/LC/A, H227Y; lane 2 is the pBAC-1/LC/A; lane 3 is the pBACgus-1/LC/A, H227Y; lane 4 is the pBACgus-1/LC/A; lane 5 is the AcNPV, vector alone, negative control; lane 6 is the Sf9 insect cells only; and lane 7 is the E. coli expressed LC/A.

FIG. 5 shows that the rLC/A expressed in BEVS was confirmed by Western Blotting. Two duplicating protein blots were probed with either anti-LC polyclonal antibody (FIG. 5A) or anti-His tag monoclonal antibody (FIG. 5B). Lane 1 is the pBAC-1/LC/A, H227Y; lane 2 is the pBAC-1/LC/A; lane 3 is the pBACgus-1/LC/A, H227Y; lane 4 is the pBACgus-1/LC/A; lane 5 is the AcNPV, negative control; lane 6 is the Sf9 insect cells only; lane M is the MagicMark, molecular marker.

FIG. 6 shows the endopeptidase enzymatic activity of baculovirally-expressed recombinant LC/A. 1 is the activity of pBAC-1/LC/A, H227Y; 2 is the activity of pBAC-1/LC/A; 3 is the activity of pBACgus-1/LC/A, H227Y; 4 is the activity of pBACgus-1/LC/A; 5 is the activity of AcNPV, negative control; 6 is the activity of Sf9 insect cell lysate only; 7 is the activity of rLC/A, positive control; and 8 is the activity of Substrate only.

FIG. 7 shows the subcloning of BoNT/A-HC into pBAC-1 or pBACgus-1 vector as confirmed by PCR. The insert of 2.6 kb was shown by PCR screening (the left panel, indicated by the arrow). It is also confirmed by restriction digestion (BamHI/XhoI) (the right panel): 2.6 kb is the insert and the slower migrated band is the vectors: either pBAC-1 or pBACgus-1.

FIG. 8 shows the PCR analysis of baculovirus recombinants: 1 is the Negative control; 2 is #6 HC/pBAC-1 transfection; and 3 is #36 HC/pBACgus-1 transfection.

FIG. 9 shows the determination of rBoNT/A HC expression by Western blotting with anti-Toxin pAb (1:5000). C is the Negative control (Baculovirus vector alone) and S is the sample from rBoNT/A HC.

FIG. 10. Both iLC and HC were expressed in Sf21 insect cells when co-infecting with iLC and HC recombinant baculovirus. Left panel: Western blot with anti-toxin A polyclonal antibody; Right panel: Western blot with anti-LC/A polyclonal antibody. Lanes: M1, Magic Marker; 1, iLC, 1 ml virus stock; 3, iBoNT/A, 1 ml virus stock; 3, iBoNT/A, 1 ml virus stock; 4, AcNPV, 1 ml virus stock; 5, iLC (1 ml) and HC (1 ml); 6, iLC (1 ml) and HC (2 ml); 7, iLC (1 ml) and HC (3 ml); 8, iLC (2 ml) and HC (1 ml); 9, iLC (3 ml) and HC (1 ml): 10, uninfected Sf21 cell lysate; M2, Seeblue Plus2 Marker.

FIG. 11. BEVS has the capacity of di-chain formation of iBoNT/A in co-infection of iLC and HC recombinant baculovirus. Left panel: Western blot with anti-toxin A polyclonal antibody; Right panel: Western blot with anti-LC/A polyclonal antibody. Lanes: M1, Magic Marker; 1, iLC, 1 ml virus stock; 3, iBoNT/A, 1 ml virus stock; 3, iBoNT/A, 1 ml virus stock; 4, AcNPV, 1 ml virus stock; 5, iLC (1 ml) and HC (1 ml); 6, iLC (1 ml) and HC (2 ml); 7, iLC (1 ml) and HC (3 ml); 8, iLC (2 ml) and HC (1 ml); 9, iLC (3 ml) and HC (1 ml): 10, uninfected Sf21 cell lysate; M2, Seeblue Plus2 Marker.

### DESCRIPTION OF EMBODIMENTS

The present invention is based, in part, upon the discovery that a BoNT light chain can form a disulfide bridge with a BoNT heavy chain in a cellular environment, thereby forming a di-chain BoNT. In some embodiments, a disulfide bridge may be formed between a cysteine residue located on the light chain and a cysteine residue located on the heavy chain.

The locations of the cysteine residues on the light chain and heavy chain are not always conserved, except for those at the C-terminus of the light chain, and the N-terminus of the heavy chain. For example, BoNT serotype A has a cysteine residue at position 431 corresponding to C-terminus of the light chain and position 454 corresponding to the N-terminus of the heavy chain; and BoNT serotype E presumably has a cysteine residue at position 412 corresponding to C-terminus of the light chain and position 426 corresponding to the N-terminus of the heavy chain).

In some embodiments, one or more disulfide bridges are formed between the light chain and the heavy chain. In some embodiments, only one disulfide bridge is formed between the light chain and the heavy chain. In some embodiments, a disulfide bridge may be formed between a cysteine residue at the C-terminus of the light chain and the N-terminus of the heavy chain. In some embodiments, a disulfide bridge may be formed between a cysteine residue at the C-terminus of the light chain and the N-terminus of the heavy chain, wherein the light chain and heavy chain are of the same serotype. For example, a cystein residue of light chain of BoNT serotype A at position 431 may form a disulfide bridge with a cysteine residue of BoNT serotype A at position 454, 791, 967, 1060 or 1280. In some embodiments, a disulfide bridge may be formed between a cysteine residue at the C-terminus of the light chain and the N-terminus of the heavy chain, wherein the light chain and heavy chain are of the same serotype, and wherein the disulfide bridge is formed between amino acid residues identical to that of the naturally existing botulinum toxin. In some embodiments, a disulfide bridge may be formed between a cysteine residue at the C-terminus of the light chain and the N-terminus of the heavy chain, wherein the light chain and heavy chain are each from a different serotype. For example, a chimera toxin may be formed with a BoNT serotype A light chain and a BoNT serotype E heavy chain, wherein the cysteine at postion 431 of the light chain forms a disulfide bridge with a cysteine at position 426 of the heavy chain. In some embodiments, a chimera toxin may be formed with a BoNT serotype E light chain and a BoNT serotype A heavy chain, wherein the cysteine at postion 412 of the light chain forms a disulfide bridge with a cysteine at position 454 of the heavy chain.

In some embodiments, a method of manufacturing a di-chain BoNT comprises expressing a BoNT light chain and a BoNT heavy chain separately in a same cell. Commonly known techniques may be employed for expressing a light chain and a heavy chain in a cell. For example, the light chain and the heavy chain may be expressed by transfecting a cell with an mRNA encoding for a light chain and an mRNA encoding for a heavy chain. Also, the light chain and the heavy chain may be expressed by transfecting a cell with a vector encoding for a light chain and heavy chain.

In some embodiments, a single vector may be used for expressing the BoNT light chain and the BoNT heavy chain in a cell. For example, a vector that is capable of expressing a light chain and a heavy chain may comprise two promoters, each followed by a coding sequence for the light chain or the heavy chain.

In some embodiments, two vectors may be used for expressing a light chain and a heavy chain in a cell. For example, a cell may be transfected with a first and a second vector, wherein the first vector expresses the light chain, and the second vector expresses the heavy chain.

In some embodiments, a vector used in accordance with this invention may be a viral-based expression vector. In some embodiments, a vector used in accordance with this invention may be a plasmid-based expression vector. The viral-based or plasmid-based expression vector may be a yeast expression vector, a bacterial expression vector, a plant expression vector, an amphibian expression vector or a mammalian expression vector.

In some embodiments, the vector is a recombinant baculovirus. The use of recombinant Baculoviruses as expression vectors is well known. Typically, the use of recombinant Baculovirus vectors involves the construction and isolation of recombinant Baculoviruses in which the coding sequence for a chosen gene, e.g., a gene encoding for a light chain or heavy chain of a BoNT, is inserted behind the promoter for a nonessential viral gene, e.g., a polyhedrin. Also, one advantage of the Baculovirus vectors over bacterial and yeast expression vectors includes the expression of recombinant proteins that are essentially authentic and are antigenitally and/or biologically active. In addition, Baculoviruses are not pathogenic to vertebrates or plants and do not employ transformed cells or transforming elements as do the mammalian expression systems. Although mammalian expression systems result in the production of fully modified, functional protein, yields are often low. E. coli systems result in high yields of recombinant protein but the protein is not modified and may be difficult to purify in a nondenatured state.

In some embodiments, a vector of the present invention comprises a baculovirus promoter operably linked to a nucleic acid sequence encoding a light chain or a heavy chain. The baculovirus expression vectors commonly employ very late promoters, such as the polyhedrin or polypeptide 10 (p10) promoters to drive foreign gene expression. These promoters are regulated during the course of virus infection and are activated very late in the infectious process usually beginning 18 to 24 hours post-infection. In some embodiments, a vector of the present invention comprises a polyhedrin promoter operably linked to a nucleic acid sequence encoding a light chain or a heavy chain.

The light chain and heavy chain may be expressed in any type of cells. In some embodiments, the light chain and heavy chain may be expressed in a prokaryotic host cell. Non-limiting examples of prokaryotic host cells include Escherichia coli cell, Clostridium botulinum cell, Clostridium tetani cell, Clostridium beratti cell, Clostridium butyricum cell, and Clostridium perfringens cell.

In some embodiments, a light chain and a heavy chain are separately expressed in an Escherichia coli cell, wherein the light chain and heavy chain form a disulfide bridge with each other after they are separately expressed in the Escherichia coli cell. An Escherichia coli cell system that may be employed include those that are disclosed by Andersen et al., Current Opinion in Biotechnology, 2002, 13: 117-123, the disclosure of which is incorporated in its entirety by reference herein.

In some embodiments, the light chain and heavy chain may be expressed in a eukaryotic host cell. Non-limiting examples of eukaryotic host cells include yeast cells, plant cells, amphibian cells, mammalian cells, and insect cells. Non-limiting examples of yeast cells include a Saccharomyces cerevisiae cell, Schizosaccharomyces pombe cell, Pichia pastoris cell, Hansenula polymorpha cell, Kluyveromyces lactis cell and Yarrowia lipolytica cell. Non-limiting example a mammalian cell includes CHO cells. Non-limiting examples of insect cell include a Spodoptera frugiperda cell (e.g., Mimic Sf9 and Sf21 Insect cell line, discussed below), Aedes albopictus cell, Trichoplusia ni cell (e.g., BTI-Tn-5B1-4 cell line), Estigmene acrea cell, Bombyx mori cell and Drosophila melanogaster cell.

The above mentioned host cells may be transfected with any expression vector operably harboring a light chain and/or heavy chain. In some embodiments, an insect cell is transfected with a baculovirus vector. Generally, an insect cell transfected with a baculovirus vector may be referred to as the baculovirus expression system (BEVS). See for example, U.S. Pat. No. 6,210,966, No. 6,090,584, No. 5,871,986, No. 5,759,809, No. 5,753,220, No. 5,750,383, No. 5,731,182, No. 5,728,580, No. 5,583,023, No. 5,571,709, No. 5,521,299, No. 5,516,657, No. 5,475,090, No. 5,472,858, No. 5,348,886, No. 5,322,774, No. 5,278,050, No. 5,244,805, No. 5,229,293, No. 5,194,376, No. 5,179,007, No. 5,169,784, No. 5,162,222, No. 5,155,037, No. 5,147,788, No. 5,110,729, No. 5,077,214, No. 5,023,328, No. 4,879,236, and No. 4,745,051. The disclosures of these reference are incorporated in their entirety by reference herein.

The baculovirus expression system is commonly used to produce recombinant proteins. A significant advantage of this system is the high expression levels-up to 250-fold greater than in mammalian expression systems, which can be achieved very rapidly. In addition, insect cells perform most of the post-translational modifications of mammalian cells, including glycosylation, and most of the proteins expressed retain biological function.

High levels of some recombinant proteins have been achieved, approaching the levels of the native polyhedrin protein from the baculovirus (1000 mg/L). However, expression of glycosylated, secreted proteins in the commonly used Spodoptera frugiperda cell lines SF9 and SF21 may be lower lower. SF9 is a clonal isolate of SF21 but in general produces about the same levels of recombinant proteins. Many secreted glycosylated proteins are produced in SF9 cells at levels below about 10 mg/L.

One of the insect cell lines that may be employed in accordance with the present invention includes the BTI-Tn-5B1-4, hereafter referred to as TN5B1-4, established at Boyce Thompson Institute, Ithaca, N.Y. and commercially available for use in research as High Five™ cells from Invitrogen Corp. The cell line is on deposit at the American serotype Culture Collection as ATCC CRL 10859. These cells were derived from eggs of the Cabbage Looper (Trichoplusia ni) and have been found to be particularly susceptible to baculoviruses, which are adaptable to genetic modifications which lead to high levels of secretion of proteins and have been shown to be superior to SF9 for expression of both cytoplasmic and secreted glycosylated proteins. TN5B1-4 optimally produced 7-fold more b-galactosidase, 26-fold more human secreted alkaline phosphatase (SEAP), and 28-fold more soluble tissue factor per cell than SF9 in monolayer cultures. However, TN5B1-4 clumps severely in suspension while SF9 does not. TN5B1-4 can be readily grown in suspension and infected at high cell density without significantly affecting their per cell production.

For cells (e.g., insect cells) that are transfected with a recombinant baculovirus, the expression of the foreign gene is usually driven by the strong polyhedrin promoter of the Autographa californica nuclear polyhedrosis virus (AcNPV) which is transcribed during the late stages of infection. The recombinant proteins are often expressed at high levels in cultured insect cells or infected larvae and are, in most cases functionally similar to their authentic counterparts.

AcNPV has a large (130 kb) circular double-stranded DNA (dsDNA) genome with multiple recognition sites for many restriction endonucleases, and as a result, recombinant baculoviruses are traditionally constructed in a two-stage process. First, a foreign gene is cloned into a plasmid downstream from a baculovirus promoter and flanked by baculovirus DNA derived from a nonessential locus, usually the polyhedrin gene. This resultant plasmid DNA, is called a transfer vector and is introduced into insect cells along with wild-type genomic viral DNA. About 1% of the resulting progeny are recombinant, with the foreign gene inserted into the genome of the parent virus by homologous recombination in vivo. The recombinant virus is purified to homogeneity by sequential plaque assays, and recombinant viruses containing the foreign gene inserted into the polyhedrin locus can be identified by an altered plaque morphology characterized by the absence of occluded virus in the nucleus of infected cells.

The construction of recombinant baculoviruses by standard transfection and plaque assay methods can take as long as four to six weeks and many methods to speed up the identification and purification of recombinant viruses have been tried in recent years. These methods include plaque lifts, serial limiting dilutions of virus and cell affinity techniques. Each of these methods require confirmation of the recombination event by visual screening of plaque morphology, DNA dot blot hybridization, immunoblotting, or amplification of specific segments of the baculovirus genome by polymerase chain reaction techniques. The identification of recombinant viruses can also be facilitated by using improved transfer vectors or through the use of improved parent viruses. Co-expression vectors are transfer vectors that contain another gene, such as the lacZ gene, under the control of a second vital or insect promoter. In this case, recombinant viruses form blue plaques when the agarose overlay in a plaque assay contains X-gal, a chromogenic substrate for .beta.-galactosidase. Although blue plaques can be identified after 3-4 days, compared to 5-6 days for optimal vizualization of occlusion minus plaques, multiple plaque assays are still required to purify the virus. It is also possible to screen for colorless plaques in a background of blue plaques, if the parent virus contains the beta-galactosidase gene at the same locus as the foreign gene in the transfer vector.

The fraction of recombinant progeny virus that results from homologous recombination between a transfer vector and a parent virus can be also be significantly improved from 0.1-1.0% to nearly 30% by using parent virus that is linearized at one or more unique sites near the target site for insertion of the foreign gene into the baculovirus genome. Linear viral DNA by itself is 15- to 150-fold less infectious than the circular viral DNA. A higher proportion of recombinant viruses (80% or higher) can be achieved using linearized viral DNA (marketed as BacPAK6, Clonetech; or as BaculoGold, Pharmingen) that is missing an essential portion of the baculovirus genome downstream from the polyhedrin gene.

Peakman et al., (1992) described the use of the Crelox sytem of bacteriophage P1 to perform cre-mediated site-specific recombination in vitro between a transfer vector and a modified parent virus that both contain the lox recombination sites. Up to 50% of the viral progeny are recombinant. Two disadvantages of this method are that there can be multiple insertions of the transfer vector into the parent virus, and that multiple plaque assays are still required to purify a recombinant virus.

A rapid method for generating recombinant baculoviruses based on homologous recombination between a baculovirus genome propagated in the yeast Saccharomyces cervisiae and a baculovirus transfer vector that contains a segment of yeast DNA is known. The shuttle vector contains a yeast ARS sequence that permits autonomous replication in yeast, a CEN sequence that contains a mitotic centromere and ensures stable segregation of plasmid DNAs into daughter cells, and two selectable marker genes (URA3 and SUP4-o) downstream from the polyhedrin promoter (Pₚₒₗₕ) in the order Pₚₒₗₕ, SUP4-o, ARS, URA3, and CEN. The transfer vector contains the foreign gene flanked on the 5′ end by baculovirus sequences and on the 3′ end by the yeast ARS sequence. Recombinant shuttle vectors which lack the SUP4-o gene can be selected in an appropriate yeast strain in the presence of a toxic amino acid analogue. Insect cells transfected with DNA isolated from selected yeast colonies produce virus and express the foreign gene under control of the polyhedrin promoter. Since all of the viral DNA isolated from yeast contains the foreign gene inserted into the baculovirus genome and there is no background of contaminating parent virus, the time-consuming steps of plaque purification are eliminated. With this method, it is possible to obtain stocks of recombinant virus within 10-12 days. Two drawbacks, however, are the relatively low transformation efficiency of S. cervisiae, and the necessity for purification of the recombinant shuttle vector DNA by sucrose gradient prior to its introduction into insect cells.

Without wishing to limit the invention to any theory or mechanism of operation, it is believed that the formation of a disulfide bridge between the light chain and heavy chain may be facilitated by one or more accessory protein. In some embodiments, the method of forming a di-chain BoNT comprises co-expressing one or more accessory protein with the light chain and heavy chain. Non-limiting examples of accessory proteins include a Nontoxic nonhemagglutinin (NTNH), hemaglutinin components (HA70, HA34, HA17), GroES, GroEL, a disulfide isomerase or a heat shock protein.

NTNH is a 130-kDa peptide which forms a complex with the BoNT after the BoNT is expressed in the anaerobic Clostridial botulinum. For BoNT/A-Hall, the NTNH may be 138 kDa. In some embodiments, the vector which operably harbors a nucleic acid sequence encoding for the light chain and/or the heavy chain also operably harbors a nucleic acid sequence encoding for the NTNH.

A light chain of the present invention include a light chain of a Clostridium botulinum toxin serotype A, B, C1, D, E, F, or G. In some embodiments, the light chain of the present invention is about 75% homologous to the nucleic acid sequence region of a Clostridium botulinum toxin serotype A, B, C1, D, E, F, or G that encodes for the light chain. In some embodiments, the light chain of the present invention is about 85% homologous to the nucleic acid sequence region of a Clostridium botulinum toxin serotype A, B, C1, D, E, F, or G that encodes for the light chain. In some embodiments, the light chain of the present invention is about 95% homologous to the nucleic acid sequence region of a Clostridium botulinum toxin serotype A, B, C1, D, E, F, or G that encodes for the light chain. Percent homology can be determined by, for example, the Gap program (Wisconsin Sequence Analysis Package, Version 8 for Unix, Genetics Computer Group, University Research Park, Madison Wis.), which uses the algorithm of Smith and Waterman (Adv. Appl. Math., 1981, 2, 482-489, which is incorporated herein by reference in its entirety) using the default settings.

In some embodiments, the light chain used in accordance with the present invention may be modified, e.g. to become inactive. For example, an active wild serotype light chain comprises a sequence encoding the zinc binding motif His-Glu-x-x-His (SEQ ID NO: 1). This wild serotype light chain may be mutated to become inactive by modifying to zinc binding motif to become Gly-Thr-x-x-Asn, (SEQ ID NO: 2), wherein x is any amino acid. See U.S. Pat. No. 6,051,239, the disclosure of which is incorporated in its entirety herein by reference. In some embodiments, a point mutant H227Y at LC of BoNT/A has been shown to abolish LC activity.

A heavy chain of the present invention may be a heavy chain of a Clostridium botulinum toxin serotypes A, B, C1, D, E, F or G. In some embodiments, the heavy chain of the present invention is about 75% homologous to the nucleic acid sequence region of a Clostridium botulinum toxin serotype A, B, C1, D, E, F, or G that encodes for the heavy chain. In some embodiments, the heavy chain of the present invention is about 85% homologous to the nucleic acid sequence region of a Clostridium botulinum toxin serotype A, B, C1, D, E, F, or G that encodes for the heavy chain. In some embodiments, the heavy chain of the present invention is about 95% homologous to the nucleic acid sequence region of a Clostridium botulinum toxin serotype A, B, C1, D, E, F, or G that encodes for the heavy chain. The nucleic acid sequences of Clostridium botulinum toxin serotype A, B, C1, D, E, F, or G are well known in the art. Further, one of ordinary skill in the art would know which regions of the nucleic acid sequence encode for the light chain and heavy chain. See, for example, Binz, T., Kurazono, H., Popoff, M. R., Eklund, M. W., Sakaguchi, G., Kozaki, S., Krieglstein, K., Henschen, A., Gill, D. M. and Niemann, H. Nucleotide sequence of the gene encoding Clostridium botulinum neurotoxin type D. Nucleic Acids Res. 18 (18), 5556 (1990); Binz, T., Kurazono, H., Wille, M., Frevert, J., Wernars, K. and Niemann, H., The complete sequence of botulinum neurotoxin type A and comparison with other clostridial neurotoxins. J. Biol. Chem. 265 (16), 9153-9158 (1990); East, A. K., Richardson, P. T., Allaway, D., Collins, M. D., Roberts, T. A., and Thompson, D. E. Sequence of the gene encoding type F neurotoxin of Clostridium botulinum. FEMS Microbiol. Lett. 96, 225-230 (1992); Campbell, K. D. (a), Collins, M. D. and East, A. K. (a) Gene probes for identification of the botulinal neurotoxin gene and specific identification of neurotoxin types B, E, and F. J. Clin. Microbiol. 31 (9), 2255-2262 (1993); Campbell, K. (b), Collins, M. D. and East, A. K. (b) Nucleotide sequence of the gene coding for Clostridium botulinum (Clostridium argentinense) type G neurotoxin: genealogical comparison with other clostridial neurotoxins. Biochim. Biophys. Acta 1216 (3), 487-491 (1993); Hutson, R. A. and Collins, M. D. The sequence of the gene encoding type F neurotoxin of clostridium botulinum NCTC 10281; Comparative analysis with other botulinal neurotoxins. Unpublished REFERENCE 2 (bases 1 to 4209) Hutson, R. A. Direct Submission. Submitted (19-Sep.-1994); Hutson, R. A., Collins, M. D., East, A. K. and Thompson, D. E. Nucleotide sequence of the gene coding for non-proteolytic Clostridium botulinum type B neurotoxin: comparison with other clostridial neurotoxins. Curr. Microbiol. 28 (2), 101-110 (1994); Kouguchi, H., Watanabe, T., Sagane, Y., Sunagawa, H. and Ohyama, T. In vitro reconstitution of the Clostridium botulinum type D progenitor toxin. J. Biol. Chem. 277 (4), 2650-2656 (2002); Moriishi, K., Koura, M., Fujii, N., Fujinaga, Y., Inoue, K., Syuto, B. and Oguma, K. Molecular cloning of the gene encoding the mosaic neurotoxin, composed of parts of botulinum neurotoxin types C1 and D, and PCR detection of this gene from Clostridium botulinum type C organisms. Appl. Environ. Microbiol. 62 (2), 662-667 (1996); Sagane, Y., Kouguchi, H., Watanabe, T., Sunagawa, H., Inoue, K., Fujinaga, Y., Oguma, K. and Ohyama, T. Role of C-terminal region of HA-33 component of botulinum toxin in hemagglutination. Biochem. Biophys. Res. Commun. 288 (3), 650-657 (2001); Moriishi, K., Koura, M., Abe, N., Fujii, N., Fujinaga, Y., Inoue, K. and Ogumad, K. Mosaic structures of neurotoxins produced from Clostridium botulinum types C and D organisms. Biochim. Biophys. Acta 1307 (2), 123-126 (1996); Poulet, S., Hauser, D., Quanz, M., Niemann, H. and Popoff, M. R. Sequences of the botulinal neurotoxin E derived from Clostridium botulinum type E (strain Beluga) and Clostridium butyricum (strains ATCC 43181 and ATCC 43755). Biochem. Biophys. Res. Commun. 183 (1), 107-113 (1992); Sagane, Y., Watanabe, T., Kouguchi, H., Yamamoto, T., Kawabe, T., Murakami, F., Nakatsuka, M. and Ohyama, T. Organization of Gene Encoding Components of the Botulinum Progenitor Toxin in Clostridium botulinum Type C Strain 6814: Evidence of Chimeric Sequence in the Gene Encoding Each Component. Published Only in DataBase (2000); Sagane, Y., Watanabe, T., Kouguchi, H., Yamamoto, T., Kawabe, T., Murakami, F., Nakatsuka, M. and Ohyama, T. Direct Submission. Submitted (17-Jan.-2000); Thompson, D. E., Brehm, J. K., Oultram, J. D., Swinfield, T. J., Shone, C. C., Atkinson, T., Melling, J. and Minton, N. P. The complete amino acid sequence of the Clostridium botulinum type A neurotoxin, deduced by nucleotide sequence analysis of the encoding gene. Eur. J. Biochem. 189 (1), 73-81 (1990); Thompson, D. E., Hutson, R. A., East, A. K., Allaway, D., Collins, M. D. and Richardson, P. T. Nucleotide sequence of the gene coding for Clostridium barati type F neurotoxin: comparison with other clostridial neurotoxins. FEMS Microbiol. Lett. 108 (2), 175-182 (1993); Whelan, S. M., Elmore, M. J., Bodsworth, N. J., Brehm, J. K., Atkinson, T. and Minton, N. P. Complete nucleotide sequence of the Clostridium botulinum gene encoding the type B neurotoxin. Unpublished (1991); Whelan, S. M., Elmore, M. J., Bodsworth, N. J., Atkinson, T. and Minton, N. P. The complete amino acid sequence of the Clostridium botulinum type-E neurotoxin, derived by nucleotide-sequence analysis of the encoding gene. Eur. J. Biochem. 204 (2), 657-667 (1992); Willems, A., East, A. K., Lawson, P. A. and Collins, M. D. Sequence of the gene coding for the neurotoxin of Clostridium botulinum type A associated with infant botulism: comparison with other clostridial neurotoxins. Res. Microbiol. 144 (7), 547-556 (1993); Zhang, L., Lin, W. J., Li, S. and Aoki, K. R. Complete DNA sequences of the botulinum neurotoxin complex of Clostridium botulinum type A-Hall (Allergan) strain. Gene 315, 21-32 (2003). The disclosures of these references are incorporated in their entirety herein by reference.

Table 1 shows the light chain and heavy chain nucleic acid sequence that may be expressed in a host cell.

TABLE 1

| | | | | |
|---|---|---|---|---|
| TOXIN | NUCLEIC ACID | SEQ | | SEQ |
| ACC NO | SEQUENCE OF LC | ID # | NUCLEIC ACID SEQUENCE OF HC | ID # |

| | | | | |
|---|---|---|---|---|
| BONT/A | ATGCCATTTGTTAATAAA | 29 | | 30 |
| AF488749 | CAATTTAATTATAAAGAT | | GCATTAAATGATTTATGTATCAAAG | |
| | CCTGTAAATGGTGTTGAT | | TTAATAATTGGGACTTGTTTTTTAG | |
| | ATTGCTTATATAAAAATT | | TCCTTCAGAAGATAATTTTACTAAT | |
| | CCAAATGCAGGACAAAT | | GATCTAAATAAAGGAGAAGAAATT | |
| | GCAACCAGTAAAAGCTTT | | ACATCTGATACTAATATAGAAGCA | |
| | TAAAATTCATAATAAAATA | | GCAGAAGAAAATATTAGTTTAGATT | |
| | TGGGTTATTCCAGAAAGA | | TAATACAACAATATTATTTAACCTT | |
| | GATACATTTACAAATCCT | | TAATTTTGATAATGAACCTGAAAAT | |
| | GAAGAAGGAGATTTAAAT | | ATTTCAATAGAAAATCTTTCAAGTG | |
| | CCACCACCAGAAGCAAA | | ACATTATAGGCCAATTAGAACTTAT | |
| | ACAAGTTCCAGTTTCATA | | GCCTAATATAGAAAGATTTCCTAAT | |
| | TTATGATTCAACATATTTA | | GGAAAAAAGTATGAGTTAGATAAA | |
| | AGTACAGATAATGAAAAA | | TATACTATGTTCCATTATCTTCGTG | |
| | GATAATTATTTAAAGGGA | | CTCAAGAATTTGAACATGGTAAAT | |
| | GTTACAAAATTATTTGAG | | CTAGGATTGCTTTAACAAATTCTGT | |
| | AGAATTTATTCAACTGAT | | TAACGAAGCATTATTAAATCCTAGT | |
| | CTTGGAAGAATGTTGTTA | | CGTGTTTATACATTTTTTTCTTCAG | |
| | ACATCAATAGTAAGGGG | | ACTATGTAAAGAAAGTTAATAAAGC | |
| | AATACCATTTTGGGGTG | | TACGGAGGCAGCTATGTTTTTAGG | |
| | GAAGTACAATAGATACAG | | CTGGGTAGAACAATTAGTATATGA | |
| | AATTAAAAGTTATTGATA | | TTTTACCGATGAAACTAGCGAAGT | |
| | CTAATTGTATTAATGTGA | | AAGTACTACGGATAAAATTGCGGA | |
| | TACAACCAGATGGTAGTT | | TATAACTATAATTATTCCATATATA | |
| | ATAGATCAGAAGAACTTA | | GGACCTGCTTTAAATATAGGTAAT | |
| | ATCTAGTAATAATAGGAC | | ATGTTATATAAAGATGATTTTGTAG | |
| | CCTCAGCTGATATTATAC | | GTGCTTTAATATTTTCAGGAGCTGT | |
| | AGTTTGAATGTAAAAGCT | | TATTCTGTTAGAATTTATACCAGAG | |
| | TTGGACATGAAGTTTTGA | | ATTGCAATACCTGTATTAGGTACTT | |
| | ATCTTACGCGAAATGGTT | | TTGCACTTGTATCATATATTGCGAA | |
| | ATGGCTCTACTCAATACA | | TAAGGTTCTAACCGTTCAAACAATA | |
| | TTAGATTTAGCCCAGATT | | GATAATGCTTTAAGTAAAAGAAATG | |
| | TTACATTTGGTTTTGAGG | | AAAAATGGGATGAGGTCTATAAAT | |
| | AGTCACTTGAAGTTGATA | | ATATAGTAACAAATTGGTTAGCAAA | |
| | CAAATCCTCTTTTAGGTG | | GGTTAATACACAGATTGATCTAATA | |
| | CAGGCAAATTTGCTACA | | AGAAAAAAAATGAAAGAAGCTTTA | |
| | GATCCAGCAGTAACATTA | | GAAAATCAAGCAGAAGCAACAAAG | |
| | GCACATGAACTTATACAT | | GCTATAATAAACTATCAGTATAATC | |
| | GCTGGACATAGATTATAT | | AATATACTGAGGAAGAGAAAAATA | |
| | GGAATAGCAATTAATCCA | | ATATTAATTTTAATATTGATGATTTA | |
| | AATAGGGTTTTTAAAGTA | | AGTTCGAAACTTAATGAGTCTATAA | |
| | AATACTAATGCCTATTAT | | ATAAAGCTATGATTAATATAAATAA | |
| | GAAATGAGTGGGTTAGA | | ATTTTTGAATCAATGCTCTGTTTCA | |
| | AGTAAGCTTTGAGGAACT | | TATTTAATGAATTCTATGATCCCTT | |
| | TAGAACATTTGGGGGAC | | ATGGTGTTAAACGGTTAGAAGATT | |
| | ATGATGCAAAGTTTATAG | | TTGATGCTAGTCTTAAAGATGCATT | |
| | ATAGTTTACAGGAAAACG | | ATTAAAGTATATATATGATAATAGA | |
| | AATTTCGTCTATATTATTA | | GGAACTTTAATTGGTCAAGTAGAT | |
| | TAATAAGTTTAAAGATAT | | AGATTAAAAGATAAAGTTAATAATA | |
| | AGCAAGTACACTTAATAA | | CACTTAGTACAGATATACCTTTTCA | |
| | AGCTAAATCAATAGTAGG | | GCTTTCCAAATACGTAGATAATCAA | |
| | TACTACTGCTTCATTACA | | AGATTATTATCTACATTTACTGAAT | |
| | GTATATGAAAAATGTTTT | | ATATTAAGAATATTATTAATACTTCT | |
| | TAAAGAGAAATATCTCCT | | ATATTGAATTTAAGATATGAAAGTA | |
| | ATCTGAAGATACATCTGG | | ATCATTTAATAGACTTATCTAGGTA | |
| | AAAATTTTCGGTAGATAA | | TGCATCAAAAATAAATATTGGTAGT | |
| | ATTAAAATTTGATAAGTT | | AAAGTAAATTTTGATCCAATAGATA | |
| | ATACAAAATGTTAACAGA | | AAAATCAAATTCAATTATTTAATTTA | |
| | GATTTACACAGAGGATAA | | GAAAGTAGTAAAATTGAGGTAATTT | |
| | TTTTGTTAAGTTTTTTAAA | | TAAAAAATGCTATTGTATATAATAG | |
| | GTACTTAACAGAAAAACA | | TATGTATGAAAATTTTAGTACTAGC | |
| | TATTTGAATTTTGATAAA | | TTTTGGATAAGAATTCCTAAGTATT | |
| | GCCGTATTTAAGATAAAT | | TTAACAGTATAAGTCTAAATAATGA | |
| | ATAGTACCTAAGGTAAAT | | ATATACAATAATAAATTGTATGGAA | |
| | TACACAATATATGATGGA | | AATAATTCAGGATGGAAAGTATCA | |
| | TTTAATTTAAGAAATACA | | CTTAATTATGGTGAAATAATCTGGA | |
| | AATTTAGCAGCAAACTTT | | CTTTACAGGATACTCAGGAAATAA | |
| | AATGGTCAAAATACAGAA | | AACAAAGAGTAGTTTTTAAATACAG | |
| | ATTAATAATATGAATTTTA | | TCAAATGATTAATATATCAGATTAT | |
| | CTAAACTAAAAAATTTTA | | ATAAACAGATGGATTTTTGTAACTA | |
| | CTGGATTGTTTGAATTTT | | TCACTAATAATAGATTAAATAACTC | |
| | ATAAGTTGCTATGTGTAA | | TAAAATTTATATAAATGGAAGATTA | |
| | GAGGGATAATAACTTCTA | | ATAGATCAAAAACCAATTTCAAATT | |
| | | | TAGGTAATATTCATGCTAGTAATAA | |
| | | | TATAATGTTTAAATTAGATGGTTGT | |
| | | | AGAGATACACATAGATATATTTGG | |
| | | | ATAAAATATTTTAATCTTTTTGATAA | |
| | | | GGAATTAAATGAAAAAGAAATCAA | |
| | | | AGATTTATATGATAATCAATCAAAT | |
| | | | TCAGGTATTTTAAAAGACTTTTGGG | |
| | | | GTGATTATTTACAATATGATAAACC | |
| | | | ATACTATATGTTAAATTTATATGAT | |
| | | | CCAAATAAATATGTCGATGTAAATA | |
| | | | ATGTAGGTATTAGAGGTTATATGTA | |
| | | | TCTTAAAGGGCCTAGAGGTAGCGT | |
| | | | AATGACTACAAACATTTATTTAAAT | |
| | | | TCAAGTTTGTATAGGGGGACAAAA | |
| | | | TTTATTATAAAAAAATATGCTTCTG | |
| | | | GAAATAAAGATAATATTGTTAGAAA | |
| | | | TAATGATCGTGTATATATTAATGTA | |
| | | | GTAGTTAAAAATAAAGAATATAGGT | |
| | | | TAGCTACTAATGCGTCACAGGCAG | |
| | | | GCGTAGAAAAAATACTAAGTGCAT | |
| | | | TAGAAATACCTGATGTAGGAAATC | |
| | | | TAAGTCAAGTAGTAGTAATGAAGT | |
| | | | CAAAAAATGATCAAGGAATAACAA | |
| | | | ATAAATGCAAAATGAATTTACAAGA | |
| | | | TAATAATGGGAATGATATAGGCTTT | |
| | | | ATAGGATTTCATCAGTTTAATAATA | |
| | | | TAGCTAAACTAGTAGCAAGTAATT | |
| | | | GGTATAATAGACAAATAGAAAGAT | |
| | | | CTAGTAGGACTTTGGGTTGCTCAT | |
| | | | GGGAATTTATTCCTGTAGATGATG | |
| | | | GATGGGGAGAAAGGCCACTGTAA | |
| BONT/B | CCAGTAACAATAAATAAT | 31 | GTACCAGGAATATGTATAGATGTA | 32 |
| 140631 | TTTAATTATAATGATCCA | | GATAATGAAAATCTTTTTTTTATAG | |
| | ATAGATAATGATAATATA | | CAGATAAAAATAGTTTTAGTGATGA | |
| | ATAATGATGGAACCACCA | | TCTTAGTAAAAATGAAAGAGTAGA | |
| | TTTGCAAGAGGAACAGG | | ATATAATACACAAAATAATTATATA | |
| | AAGATATTATAAAGCATT | | GGAAATGATTTTCCAATAAATGAAC | |
| | TAAAATAACAGATAGAAT | | TTATACTTGATACAGATCTTATAAG | |
| | ATGGATAATACCAGAAAG | | TAAAATAGAACTTCCAAGTGAAAAT | |
| | ATATACATTTGGATATAA | | ACAGAAAGTCTTACAGATTTTAATG | |
| | ACCAGAAGATTTTAATAA | | TAGATGTACCAGTATATGAAAAAC | |
| | AAGTAGTGGAATATTTAA | | AACCAGCAATAAAAAAAGTATTTAC | |
| | TAGAGATGTATGTGAATA | | AGATGAAAATACAATATTTCAATAT | |
| | TTATGATCCAGATTATCT | | CTTTATAGTCAAACATTTCCACTTA | |
| | TAATACAAATGATAAAAA | | ATATAAGAGATATAAGTCTTACAAG | |
| | AAATATATTTTTTCAAACA | | TAGTTTTGATGATGCACTTCTTGTA | |
| | CTTATAAAACTTTTTAATA | | AGTAGTAAAGTATATAGTTTTTTTA | |
| | GAATAAAAAGTAAACCAC | | GTATGGATTATATAAAAACAGCAAA | |
| | TTGGAGAAAAACTTCTTG | | TAAAGTAGTAGAAGCAGGACTTTT | |
| | AAATGATAATAAATGGAA | | TGCAGGATGGGTAAAACAAATAGT | |
| | TACCATATCTTGGAGATA | | AGATGATTTTGTAATAGAAGCAAAT | |
| | GAAGAGTACCACTTGAA | | AAAAGTAGTACAATGGATAAAATA | |
| | GAATTTAATACAAATATA | | GCAGATATAAGTCTTATAGTACCAT | |
| | GCAAGTGTAACAGTAAAT | | ATATAGGACTTGCACTTAATGTAG | |
| | AAACTTATAAGTAATCCA | | GAGATGAAACAGCAAAAGGAAATT | |
| | GGAGAAGTAGAAAGAAA | | TTGAAAGTGCATTTGAAATAGCAG | |
| | AAAAGGAATATTTGCAAA | | GAAGTAGTATACTTCTTGAATTTAT | |
| | TCTTATAATATTTGGACC | | ACCAGAACTTCTTATACCAGTAGT | |
| | AGGACCAGTACTTAATGA | | AGGAGTATTTCTTCTTGAAAGTTAT | |
| | AAATGAAACAATAGATAT | | ATAGATAATAAAAATAAAATAATAA | |
| | AGGAATACAAAATCATTT | | AAACAATAGATAATGCACTTACAAA | |
| | TGCAAGTAGAGAAGGAT | | AAGAGTAGAAAAATGGATAGATAT | |
| | TTGGAGGAATAATGCAAA | | GTATGGACTTATAGTAGCACAATG | |
| | TGAAATTTTGTCCAGAAT | | GCTTAGTACAGTAAATACACAATTT | |
| | ATGTAAGTGTATTTAATA | | TATACAATAAAAGAAGGAATGTATA | |
| | ATGTACAAGAAAATAAAG | | AAGCACTTAATTATCAAGCACAAG | |
| | GAGCAAGTATATTTAATA | | CACTTGAAGAAATAATAAAATATAA | |
| | GAAGAGGATATTTTAGTG | | ATATAATATATATAGTGAAGAAGAA | |
| | ATCCAGCACTTATACTTA | | AAAAGTAATATAAATATAAATTTTA | |
| | TGCATGAACTTATACATG | | ATGATATAAATAGTAAACTTAATGA | |
| | TACTTCATGGACTTTATG | | TGGAATAAATCAAGCAATGGATAA | |
| | GAATAAAAGTAGATGATC | | TATAAATGATTTTATAAATGAATGT | |
| | TTCCAATAGTACCAAATG | | AGTGTAAGTTATCTTATGAAAAAAA | |
| | AAAAAAAATTTTTTATGC | | TGATACCACTTGCAGTAAAAAAAC | |
| | AAAGTACAGATACAATAC | | TTCTTGATTTTGATAATACACTTAA | |
| | AAGCAGAAGAACTTTATA | | AAAAAATCTTCTTAATTATATAGAT | |
| | CATTTGGAGGACAAGAT | | GAAAATAAACTTTATCTTATAGGAA | |
| | CCAAGTATAATAAGTCCA | | GTGTAGAAGATGAAAAAAGTAAAG | |
| | AGTACAGATAAAAGTATA | | TAGATAAATATCTTAAAACAATAAT | |
| | TATGATAAAGTACTTCAA | | ACCATTTGATCTTAGTACATATAGT | |
| | AATTTTAGAGGAATAGTA | | AATATAGAAATACTTATAAAAATAT | |
| | GATAGACTTAATAAAGTA | | TTAATAAATATAATAGTGAAATACT | |
| | CTTGTATGTATAAGTGAT | | TAATAATATAATACTTAATCTTAGA | |
| | CCAAATATAAATATAAAT | | TATAGAGATAATAATCTTATAGATC | |
| | ATATATAAAAATAAATTTA | | TTAGTGGATATGGAGCAAAAGTAG | |
| | AAGATAAATATAAATTTG | | AAGTATATGATGGAGTAAAACTTAA | |
| | TAGAAGATAGTGAAGGA | | TGATAAAAATCAATTTAAACTTACA | |
| | AAATATAGTATAGATGTA | | AGTAGTGCAGATAGTAAAATAAGA | |
| | GAAAGTTTTAATAAACTT | | GTAACACAAAATCAAAATATAATAT | |
| | TATAAAAGTCTTATGCTT | | TTAATAGTATGTTTCTTGATTTTAG | |
| | GGATTTACAGAAATAAAT | | TGTAAGTTTTTGGATAAGAATACCA | |
| | ATAGCAGAAAATTATAAA | | AAATATAGAAATGATGATATACAAA | |
| | ATAAAAACAAGAGCAAGT | | ATTATATACATAATGAATATACAAT | |
| | TATTTTAGTGATAGTCTT | | AATAAATTGTATGAAAAATAATAGT | |
| | CCACCAGTAAAAATAAAA | | GGATGGAAAATAAGTATAAGAGGA | |
| | AATCTTCTTGATAATGAA | | AATAGAATAATATGGACACTTATAG | |
| | ATATATACAATAGAAGAA | | ATATAAATGGAAAAACAAAAAGTGT | |
| | GGATTTAATATAAGTGAT | | ATTTTTTGAATATAATATAAGAGAA | |
| | AAAAATATGGGAAAAGAA | | GATATAAGTGAATATATAAATAGAT | |
| | TATAGAGGACAAAATAAA | | GGTTTTTTGTAACAATAACAAATAA | |
| | GCAATAAATAAACAAGCA | | TCTTGATAATGCAAAAATATATATA | |
| | TATGAAGAAATAAGTAAA | | AATGGAACACTTGAAAGTAATATG | |
| | GAACATCTTGCAGTATAT | | GATATAAAAGATATAGGAGAAGTA | |
| | AAAATACAAATGTGTAAA | | ATAGTAAATGGAGAAATAACATTTA | |
| | AGTGTAAAA | | AACTTGATGGAGATGTAGATAGAA | |
| | | | CACAATTTATATGGATGAAATATTT | |
| | | | TAGTATATTTAATACACAACTTAAT | |
| | | | CAAAGTAATATAAAAGAAATATATA | |
| | | | AAATACAAAGTTATAGTGAATATCT | |
| | | | TAAAGATTTTTGGGGAAATCCACTT | |
| | | | ATGTATAATAAAGAATATTATATGT | |
| | | | TTAATGCAGGAAATAAAAATAGTTA | |
| | | | TATAAAACTTGTAAAAGATAGTAGT | |
| | | | GTAGGAGAAATACTTATAAGAAGT | |
| | | | AAATATAATCAAAATAGTAATTATA | |
| | | | TAAATTATAGAAATCTTTATATAGG | |
| | | | AGAAAAATTTATAATAAGAAGAGAA | |
| | | | AGTAATAGTCAAAGTATAAATGATG | |
| | | | ATATAGTAAGAAAAGAAGATTATAT | |
| | | | ACATCTTGATCTTGTACTTCATCAT | |
| | | | GAAGAATGGAGAGTATATGCATAT | |
| | | | AAATATTTTAAAGAACAAGAAGAAA | |
| | | | AACTTTTTCTTAGTATAATAAGTGA | |
| | | | TAGTAATGAATTTTATAAAACAATA | |
| | | | GAAATAAAAGAATATGATGAACAA | |
| | | | CCAAGTTATAGTTGTCAACTTCTTT | |
| | | | TTAAAAAAGATGAAGAAAGTACAG | |
| | | | ATGATATAGGACTTATAGGAATAC | |
| | | | ATAGATTTTATGAAAGTGGAGTACT | |
| | | | TAGAAAAAAATATAAAGATTATTTT | |
| | | | TGTATAAGTAAATGGTATCTTAAAG | |
| | | | AAGTAAAAAGAAAACCATATAAAA | |
| | | | GTAATCTTGGATGTAATTGGCAATT | |
| | | | TATACCAAAAGATGAAGGATGGAC | |
| | | | AGAA | |
| BONT/C1 | CCAATAACAATAAATAAT | 33 | ACACTTGATTGTAGAGAACTTCTT | 34 |
| P18640 | TTTAATTATAGTGATCCA | | GTAAAAAATACAGATCTTCCATTTA | |
| | GTAGATAATAAAAATATA | | TAGGAGATATAAGTGATGTAAAAA | |
| | CTTTATCTTGATACACAT | | CAGATATATTTCTTAGAAAAGATAT | |
| | CTTAATACACTTGCAAAT | | AAATGAAGAAACAGAAGTAATATAT | |
| | GAACCAGAAAAAGCATTT | | TATCCAGATAATGTAAGTGTAGAT | |
| | AGAATAACAGGAAATATA | | CAAGTAATACTTAGTAAAAATACAA | |
| | TGGGTAATACCAGATAG | | GTGAACATGGACAACTTGATCTTC | |
| | ATTTAGTAGAAATAGTAA | | TTTATCCAAGTATAGATAGTGAAAG | |
| | TCCAAATCTTAATAAACC | | TGAAATACTTCCAGGAGAAAATCA | |
| | ACCAAGAGTAACAAGTC | | AGTATTTTATGATAATAGAACACAA | |
| | CAAAAAGTGGATATTATG | | AATGTAGATTATCTTAATAGTTATT | |
| | ATCCAAATTATCTTAGTA | | ATTATCTTGAAAGTCAAAAACTTAG | |
| | CAGATAGTGATAAAGATC | | TGATAATGTAGAAGATTTTACATTT | |
| | CATTTCTTAAAGAAATAA | | ACAAGAAGTATAGAAGAAGCACTT | |
| | TAAAACTTTTTAAAAGAA | | GATAATAGTGCAAAAGTATATACAT | |
| | TAAATAGTAGAGAAATAG | | ATTTTCCAACACTTGCAAATAAAGT | |
| | GAGAAGAACTTATATATA | | AAATGCAGGAGTACAAGGAGGACT | |
| | GACTTAGTACAGATATAC | | TTTTCTTATGTGGGCAAATGATGTA | |
| | CATTTCCAGGAAATAATA | | GTAGAAGATTTTACAACAAATATAC | |
| | ATACACCAATAAATACAT | | TTAGAAAAGATACACTTGATAAAAT | |
| | TTGATTTTGATGTAGATT | | AAGTGATGTAAGTGCAATAATACC | |
| | TTAATAGTGTAGATGTAA | | ATATATAGGACCAGCACTTAATATA | |
| | AAACAAGACAAGGAAATA | | AGTAATAGTGTAAGAAGAGGAAAT | |
| | ATTGGGTAAAAACAGGA | | TTTACAGAAGCATTTGCAGTAACA | |
| | AGTATAAATCCAAGTGTA | | GGAGTAACAATACTTCTTGAAGCA | |
| | ATAATAACAGGACCAAGA | | TTTCCAGAATTTACAATACCAGCAC | |
| | GAAAATATAATAGATCCA | | TTGGAGCATTTGTAATATATAGTAA | |
| | GAAACAAGTACATTTAAA | | AGTACAAGAAAGAAATGAAATAAT | |
| | CTTACAAATAATACATTT | | AAAAACAATAGATAATTGTCTTGAA | |
| | GCAGCACAAGAAGGATT | | CAAAGAATAAAAAGATGGAAAGAT | |
| | TGGAGCACTTAGTATAAT | | AGTTATGAATGGATGATGGGAACA | |
| | AAGTATAAGTCCAAGATT | | TGGCTTAGTAGAATAATAACACAAT | |
| | TATGCTTACATATAGTAA | | TTAATAATATAAGTTATCAAATGTA | |
| | TGCAACAAATGATGTAG | | TGATAGTCTTAATTATCAAGCAGG | |
| | GAGAAGGAAGATTTAGT | | AGCAATAAAAGCAAAAATAGATCTT | |
| | AAAAGTGAATTTTGTATG | | GAATATAAAAAATATAGTGGAAGT | |
| | GATCCAATACTTATACTT | | GATAAAGAAAATATAAAAAGTCAA | |
| | ATGCATGAACTTAATCAT | | GTAGAAAATCTTAAAAATAGTCTTG | |
| | GCAATGCATAATCTTTAT | | ATGTAAAAATAAGTGAAGCAATGA | |
| | GGAATAGCAATACCAAAT | | ATAATATAAATAAATTTATAAGAGA | |
| | GATCAAACAATAAGTAGT | | ATGTAGTGTAACATATCTTTTTAAA | |
| | GTAACAAGTAATATATTT | | AATATGCTTCCAAAAGTAATAGATG | |
| | TATAGTCAATATAATGTA | | AACTTAATGAATTTGATAGAAATAC | |
| | AAACTTGAATATGCAGAA | | AAAAGCAAAACTTATAAATCTTATA | |
| | ATATATGCATTTGGAGGA | | GATAGTCATAATATAATACTTGTAG | |
| | CCAACAATAGATCTTATA | | GAGAAGTAGATAAACTTAAAGCAA | |
| | CCAAAAAGTGCAAGAAA | | AAGTAAATAATAGTTTTCAAAATAC | |
| | ATATTTTGAAGAAAAAGC | | AATACCATTTAATATATTTAGTTATA | |
| | ACTTGATTATTATAGAAG | | CAAATAATAGTCTTCTTAAAGATAT | |
| | TATAGCAAAAAGACTTAA | | AATAAATGAATATTTTAATAATATAA | |
| | TAGTATAACAACAGCAAA | | ATGATAGTAAAATACTTAGTCTTCA | |
| | TCCAAGTAGTTTTAATAA | | AAATAGAAAAAATACACTTGTAGAT | |
| | ATATATAGGAGAATATAA | | ACAAGTGGATATAATGCAGAAGTA | |
| | ACAAAAACTTATAAGAAA | | AGTGAAGAAGGAGATGTACAACTT | |
| | ATATAGATTTGTAGTAGA | | AATCCAATATTTCCATTTGATTTTA | |
| | AAGTAGTGGAGAAGTAA | | AACTTGGAAGTAGTGGAGAAGATA | |
| | CAGTAAATAGAAATAAAT | | GAGGAAAAGTAATAGTAACACAAA | |
| | TTGTAGAACTTTATAATG | | ATGAAAAATATAGTATATAATAGTAT | |
| | AACTTACACAAATATTTA | | GTATGAAAGTTTTAGTATAAGTTTT | |
| | CAGAATTTAATTATGCAA | | TGGATAAGAATAAATAAATGGGTA | |
| | AAATATATAATGTACAAA | | AGTAATCTTCCAGGATATACAATAA | |
| | ATAGAAAAATATATCTTA | | TAGATAGTGTAAAAAATAATAGTG | |
| | GTAATGTATATACACCAG | | GATGGAGTATAGGAATAATAAGTA | |
| | TAACAGCAAATATACTTG | | ATTTTCTTGTATTTACACTTAAACA | |
| | ATGATAATGTATATGATA | | AAATGAAGATAGTGAACAAAGTAT | |
| | TACAAAATGGATTTAATA | | AAATTTTAGTTATGATATAAGTAAT | |
| | TACCAAAAAGTAATCTTA | | AATGCACCAGGATATAATAAATGG | |
| | ATGTACTTTTTATGGGAC | | TTTTTTGTAACAGTAACAAATAATA | |
| | AAAATCTTAGTAGAAATC | | TGATGGGAAATATGAAAATATATAT | |
| | CAGCACTTAGAAAAGTAA | | AAATGGAAAACTTATAGATACAATA | |
| | ATCCAGAAAATATGCTTT | | AAAGTAAAAGAACTTACAGGAATA | |
| | ATCTTTTTACAAAATTTTG | | AATTTTAGTAAAACAATAACATTTG | |
| | TCATAAAGCAATAGATGG | | AAATAAATAAAATACCAGATACAG | |
| | AAGAAGTCTTTATAATAA | | GACTTATAACAAGTGATAGTGATA | |
| | A | | ATATAAATATGTGGATAAGAGATTT | |
| | | | TTATATATTTGCAAAAGAACTTGAT | |
| | | | GGAAAAGATATAAATATACTTTTTA | |
| | | | ATAGTCTTCAATATACAAATGTAGT | |
| | | | AAAAGATTATTGGGGAAATGATCT | |
| | | | TAGATATAATAAAGAATATTATATG | |
| | | | GTAAATATAGATTATCTTAATAGAT | |
| | | | ATATGTATGCAAATAGTAGACAAAT | |
| | | | AGTATTTAATACAAGAAGAAATAAT | |
| | | | AATGATTTTAATGAAGGATATAAAA | |
| | | | TAATAATAAAAAGAATAAGAGGAAA | |
| | | | TACAAATGATACAAGAGTAAGAGG | |
| | | | AGGAGATATACTTTATTTTGATATG | |
| | | | ACAATAAATAATAAAGCATATAATC | |
| | | | TTTTTATGAAAAATGAAACAATGTA | |
| | | | TGCAGATAATCATAGTACAGAAGA | |
| | | | TATATATGCAATAGGACTTAGAGA | |
| | | | ACAAACAAAAGATATAAATGATAAT | |
| | | | ATAATATTTCAAATACAACCAATGA | |
| | | | ATAATACATATTATTATGCAAGTCA | |
| | | | AATATTTAAAAGTAATTTTAATGGA | |
| | | | GAAAATATAAGTGGAATATGTAGT | |
| | | | ATAGGAACATATAGATTTAGACTTG | |
| | | | GAGGAGATTGGTATAGACATAATT | |
| | | | ATCTTGTACCAACAGTAAAACAAG | |
| | | | GAAATTATGCAAGTCTTCTTGAAA | |
| | | | GTACAAGTACACATTGGGGATTTG | |
| | | | TACCAGTAAGTGAA | |
| BONT/D | ATGACATGGCCAGTAAA | 35 | AATAGTAGAGATGATAGTACATGT | 36 |
| P19321 | AGATTTTAATTATAGTGA | | ATAAAAGTAAAAAATAATAGACTTC | |
| | TCCAGTAAATGATAATGA | | CATATGTAGCAGATAAAGATAGTA | |
| | TATACTTTATCTTAGAATA | | TAAGTCAAGAAATATTTGAAAATAA | |
| | CCACAAAATAAACTTATA | | AATAATAACAGATGAAACAAATGTA | |
| | ACAACACCAGTAAAAGC | | CAAAATTATAGTGATAAATTTAGTC | |
| | ATTTATGATAACACAAAA | | TTGATGAAAGTATACTTGATGGAC | |
| | TATATGGGTAATACCAGA | | AAGTACCAATAAATCCAGAAATAG | |
| | AAGATTTAGTAGTGATAC | | TAGATCCACTTCTTCCAAATGTAAA | |
| | AAATCCAAGTCTTAGTAA | | TATGGAACCACTTAATCTTCCAGG | |
| | ACCACCAAGACCAACAA | | AGAAGAAATAGTATTTTATGATGAT | |
| | GTAAATATCAAAGTTATT | | ATAACAAAATATGTAGATTATCTTA | |
| | ATGATCCAAGTTATCTTA | | ATAGTTATTATTATCTTGAAAGTCA | |
| | GTACAGATGAACAAAAA | | AAAACTTAGTAATAATGTAGAAAAT | |
| | GATACATTTCTTAAAGGA | | ATAACACTTACAACAAGTGTAGAA | |
| | ATAATAAAACTTTTTAAAA | | GAAGCACTTGGATATAGTAATAAA | |
| | GAATAAATGAAAGAGATA | | ATATATACATTTCTTCCAAGTCTTG | |
| | TAGGAAAAAAACTTATAA | | CAGAAAAAGTAAATAAAGGAGTAC | |
| | ATTATCTTGTAGTAGGAA | | AAGCAGGACTTTTTCTTAATTGGG | |
| | GTCCATTTATGGGAGATA | | CAAATGAAGTAGTAGAAGATTTTA | |
| | GTAGTACACCAGAAGAT | | CAACAAATATAATGAAAAAAGATAC | |
| | ACATTTGATTTTACAAGA | | ACTTGATAAAATAAGTGATGTAAGT | |
| | CATACAACAAATATAGCA | | GTAATAATACCATATATAGGACCA | |
| | GTAGAAAAATTTGAAAAT | | GCACTTAATATAGGAAATAGTGCA | |
| | GGAAGTTGGAAAGTAAC | | CTTAGAGGAAATTTTAATCAAGCAT | |
| | AAATATAATAACACCAAG | | TTGCAACAGCAGGAGTAGCATTTC | |
| | TGTACTTATATTTGGACC | | TTCTTGAAGGATTTCCAGAATTTAC | |
| | ACTTCCAAATATACTTGA | | AATACCAGCACTTGGAGTATTTAC | |
| | TTATACAGCAAGTCTTAC | | ATTTTATAGTAGTATACAAGAAAGA | |
| | ACTTCAAGGACAACAAA | | GAAAAAATAATAAAAACAATAGAAA | |
| | GTAATCCAAGTTTTGAAG | | ATTGTCTTGAACAAAGAGTAAAAA | |
| | GATTTGGAACACTTAGTA | | GATGGAAAGATAGTTATCAATGGA | |
| | TACTTAAAGTAGCACCAG | | TGGTAAGTAATTGGCTTAGTAGAA | |
| | AATTTCTTCTTACATTTAG | | TAACAACACAATTTAATCATATAAA | |
| | TGATGTAACAAGTAATCA | | TTATCAAATGTATGATAGTCTTAGT | |
| | AAGTAGTGCAGTACTTG | | TATCAAGCAGATGCAATAAAAGCA | |
| | GAAAAAGTATATTTTGTA | | AAAATAGATCTTGAATATAAAAAAT | |
| | TGGATCCAGTAATAGCA | | ATAGTGGAAGTGATAAAGAAAATA | |
| | CTTATGCATGAACTTACA | | TAAAAAGTCAAGTAGAAAATCTTAA | |
| | CATAGTCTTCATCAACTT | | AAATAGTCTTGATGTAAAAATAAGT | |
| | TATGGAATAAATATACCA | | GAAGCAATGAATAATATAAATAAAT | |
| | AGTGATAAAAGAATAAGA | | TTATAAGAGAATGTAGTGTAACATA | |
| | CCACAAGTAAGTGAAGG | | TCTTTTTAAAAATATGCTTCCAAAA | |
| | ATTTTTTAGTCAAGATGG | | GTAATAGATGAACTTAATAAATTTG | |
| | ACCAAATGTACAATTTGA | | ATCTTAGAACAAAAACAGAACTTAT | |
| | AGAACTTTATACATTTGG | | AAATCTTATAGATAGTCATAATATA | |
| | AGGACTTGATGTAGAAAT | | ATACTTGTAGGAGAAGTAGATAGA | |
| | AATACCACAAATAGAAAG | | CTTAAAGCAAAAGTAAATGAAAGTT | |
| | AAGTCAACTTAGAGAAAA | | TTGAAAATACAATGCCATTTAATAT | |
| | AGCACTTGGACATTATAA | | ATTTAGTTATACAAATAATAGTCTT | |
| | AGATATAGCAAAAAGACT | | CTTAAAGATATAATAAATGAATATT | |
| | TAATAATATAAATAAAAC | | TTAATAGTATAAATGATAGTAAAAT | |
| | AATACCAAGTAGTTGGAT | | ACTTAGTCTTCAAAATAAAAAAAAT | |
| | AAGTAATATAGATAAATA | | GCACTTGTAGATACAAGTGGATAT | |
| | TAAAAAAATATTTAGTGA | | AATGCAGAAGTAAGAGTAGGAGAT | |
| | AAAATATAATTTTGATAAA | | AATGTACAACTTAATACAATATATA | |
| | GATAATACAGGAAATTTT | | CAAATGATTTTAAACTTAGTAGTAG | |
| | GTAGTAAATATAGATAAA | | TGGAGATAAAATAATAGTAAATCTT | |
| | TTTAATAGTCTTTATAGT | | AATAATAATATACTTTATAGTGCAA | |
| | GATCTTACAAATGTAATG | | TATATGAAAATAGTAGTGTAAGTTT | |
| | AGTGAAGTAGTATATAGT | | TTGGATAAAAATAAGTAAAGATCTT | |
| | AGTCAATATAATGTAAAA | | ACAAATAGTCATAATGAATATACAA | |
| | AATAGAACACATTATTTT | | TAATAAATAGTATAGAACAAAATAG | |
| | AGTAGACATTATCTTCCA | | TGGATGGAAACTTTGTATAAGAAA | |
| | GTATTTGCAAATATACTT | | TGGAAATATAGAATGGATACTTCA | |
| | GATGATAATATATATACA | | AGATGTAAATAGAAAATATAAAAGT | |
| | ATAAGAGATGGATTTAAT | | CTTATATTTGATTATAGTGAAAGTC | |
| | CTTACAAATAAAGGATTT | | TTAGTCATACAGGATATACAAATAA | |
| | AATATAGAAAATAGTGGA | | ATGGTTTTTTGTAACAATAACAAAT | |
| | CAAAATATAGAAAGAAAT | | AATATAATGGGATATATGAAACTTT | |
| | CCAGCACTTCAAAAACTT | | ATATAAATGGAGAACTTAAACAAA | |
| | AGTAGTGAAAGTGTAGTA | | GTCAAAAAATAGAAGATCTTGATG | |
| | GATCTTTTTACAAAAGTA | | AAGTAAAACTTGATAAAACAATAGT | |
| | TGTCTTAGACTTACAAAA | | ATTTGGAATAGATGAAAATATAGAT | |
| | | | GAAAATCAAATGCTTTGGATAAGA | |
| | | | GATTTTAATATATTTAGTAAAGAAC | |
| | | | TTAGTAATGAAGATATAAATATAGT | |
| | | | ATATGAAGGACAAATACTTAGAAAT | |
| | | | GTAATAAAAGATTATTGGGGAAAT | |
| | | | CCACTTAAATTTGATACAGAATATT | |
| | | | ATATAATAAATGATAATTATATAGA | |
| | | | TAGATATATAGCACCAGAAAGTAA | |
| | | | TGTACTTGTACTTGTACAATATCCA | |
| | | | GATAGAAGTAAACTTTATACAGGA | |
| | | | AATCCAATAACAATAAAAAGTGTAA | |
| | | | GTGATAAAAATCCATATAGTAGAAT | |
| | | | ACTTAATGGAGATAATATAATACTT | |
| | TTGAGATTAAATTCTCAA | | ATCAGCATCGTCGTGCCCTACATT | |
| | ATGGTAGCCAAGACATA | | GGTTTGGCATTAAACATTGGTAAT | |
| | CTATTACCTAATGTTATT | | GAGGCGCAAAAGGGGAACTTTAAA | |
| | ATAATGGGAGCAGAGCC | | GACGCCCTGGAATTATTAGGAGCA | |
| | TGATTTATTTGAAACTAA | | GGTATTCTGCTGGAGTTCGAACCT | |
| | CAGTTCCAATATTTCTCT | | GAGCTGCTGATTCCGACTATTTTA | |
| | AAGAAATAATTATATGCC | | GTGTTCACCATTAAATCCTTCTTAG | |
| | AAGCAATCACGGTTTTG | | GCTCTAGTGACAACAAAAATAAAG | |
| | GATCAATAGCTATAGTAA | | TGATTAAAGCGATCAATAATGCCC | |
| | CATTCTCACCTGAATATT | | TTAAAGAACGTGATGAGAAATGGA | |
| | CTTTTAGATTTAATGATA | | AAGAAGTCTACTCCTTCATTGTCTC | |
| | ATAGTATGAATGAATTTA | | AAATTGGATGACGAAAATCAACAC | |
| | TTCAAGATCCTGCTCTTA | | GCAGTTTAATAAACGCAAAGAACA | |
| | CATTAATGCATGAATTAA | | GATGTATCAGGCGCTGCAAAACCA | |
| | TACATTCATTACATGGAC | | GGTTAATGCGATCAAGACAATTAT | |
| | TATATGGGGCTAAAGGG | | TGAATCTAAGTACAACTCGTACAC | |
| | ATTACTACAAAGTATACT | | CCTGGAGGAGAAAAATGAACTGAC | |
| | ATAACACAAAAACAAAAT | | TAATAAGTACGATATTAAACAAATC | |
| | CCCCTAATAACAAATATA | | GAAAACGAATTGAATCAGAAAGTC | |
| | AGAGGTACAAATATTGAA | | TCCATCGCTATGAACAATATCGAT | |
| | GAATTCTTAACTTTTGGA | | CGCTTTCTGACCGAAAGCTCTATT | |
| | GGTACTGATTTAAACATT | | TCCTATTTGATGAAACTTATCAATG | |
| | ATTACTAGTGCTCAGTCC | | AAGTCAAAATCAACAAACTTCGCG | |
| | AATGATATCTATACTAAT | | AATATGATGAGAACGTAAAAACGT | |
| | CTTCTAGCTGATTATAAA | | ACCTGCTCAATTATATTATTCAACA | |
| | AAAATAGCGTCTAAACTT | | TGGGTCGATTCTGGGCGAGTCTCA | |
| | AGCAAAGTACAAGTATCT | | ACAAGAATTGAACTCGATGGTGAC | |
| | AATCCACTACTTAATCCT | | GGATACTTTGAATAACTCGATTCC | |
| | TATAAAGATGTTTTTGAA | | GTTTAAATTATCGTCATACACCGAT | |
| | GCAAAGTATGGATTAGAT | | GATAAAATTCTTATCTCGTACTTCA | |
| | AAAGATGCTAGCGGAAT | | ACAAATTCTTTAAGCGGATCAAAA | |
| | TTATTCGGTAAATATAAA | | GCAGCAGCGTCCTTAATATGCGCT | |
| | CAAATTTAATGATATTTTT | | ATAAAAACGATAAGTACGTAGATA | |
| | AAAAAATTATACAGCTTT | | CGTCTGGATACGACAGTAACATTA | |
| | ACGGAATTTGATTTAGCA | | ATATTAATGGGGACGTCTATAAATA | |
| | ACTAAATTTCAAGTTAAA | | TCCGACAAATAAAAACCAATTCGG | |
| | TGTAGGCAAACTTATATT | | GATTTATAATGATAAACTTTCGGAG | |
| | GGACAGTATAAATACTTC | | GTGAACATCAGCCAGAACGATTAT | |
| | AAACTTTCAAACTTGTTA | | ATTATTTACGATAATAAATACAAAA | |
| | AATGATTCTATTTATAATA | | ACTTCAGCATTTCTTTTTGGGTGC | |
| | TATCAGAAGGCTATAATA | | GTATCCCAAATTACGACAACAAAA | |
| | TAAATAATTTAAAGGTAA | | TTGTGAACGTGAATAACGAATACA | |
| | ATTTTAGAGGACAGAATG | | CGATCATTAATTGCATGCGCGATA | |
| | CAAATTTAAATCCTAGAA | | ACAATTCTGGTTGGAAAGTTAGCC | |
| | TTATTACACCAATTACAG | | TGAATCACAATGAGATTATCTGGA | |
| | GTAGAGGACTAGTAAAA | | CTCTTCAGGACAATGCTGGTATCA | |
| | AAAATCATTAGATTTTGT | | ACCAAAAATTAGCGTTCAACTACG | |
| | AAAAATATTGTTTCTGTA | | GTAATGCCAACGGTATTTCTGACT | |
| | AAAGGCATAAGGA | | ACATCAATAAGTGGATCTTTGTGA | |
| | | | CCATCACCAATGACCGCCTCGGC | |
| | | | GATAGCAAGCTGTACATTAACGGT | |
| | | | AACCTGATCGACCAGAAATCTATT | |
| | | | CTGAACCTGGGTAACATTCACGTA | |
| | | | AGTGACAACATCCTTTTTAAAATTG | |
| | | | TCAATTGCTCGTATACTCGTTATAT | |
| | | | CGGCATTCGCTATTTCAATATTTTC | |
| | | | GACAAAGAACTGGATGAGACGGA | |
| | | | AATCCAGACTCTGTATTCTAACGA | |
| | | | ACCGAACACCAACATCCTGAAGGA | |
| | | | CTTTTGGGGGAATTATCTTCTCTAC | |
| | | | GATAAAGAGTACTACCTTCTTAAC | |
| | | | GTGTTGAAGCCGAACAACTTCATT | |
| | | | GATCGTCGTAAGGATAGCACCTTG | |
| | | | AGCATTAACAACATTCGTAGCACC | |
| | | | ATTTTACTGGCAAACCGCCTGTAC | |
| | | | AGCGGCATTAAAGTCAAAATTCAG | |
| | | | CGTGTCAATAACTCCAGTACGAAT | |
| | | | GACAATCTGGTGCGGAAAAATGAC | |
| | | | CAAGTCTATATTAACTTTGTCGCAA | |
| | | | GCAAAACTCACCTCTTTCCATTATA | |
| | | | TGCGGATACAGCTACCACCAATAA | |
| | | | AGAAAAAACTATTAAAATCTCCTCT | |
| | | | TCCGGGAACCGCTTTAATCAGGTG | |
| | | | GTAGTTATGAACTCGGTCGGCAAC | |
| | | | AATTGTACTATGAATTTTAAAAATA | |
| | | | ATAACGGCAATAACATCGGCCTGC | |
| | | | TGGGCTTCAAAGCTGATACAGTTG | |
| | | | TGGCCAGCACCTGGTATTACACCC | |
| | | | ACATGCGTGATCATACCAATAGTA | |
| | | | ATGGCTGCTTTTGGAATTTTATTTC | |
| | | | TGAAGAGCACGGCTGGCAAGAAA | |
| | | | AA | |
| BONT/F | ATGCCAGTAGCAATAAAT | 39 | GGAACAAAAGCACCACCAAGACTT | 40 |
| P30996 | AGTTTTAATTATAATGAT | | TGTATAAGAGTAAATAATAGTGAAC | |
| | CCAGTAAATGATGATACA | | TTTTTTTTGTAGCAAGTGAAAGTAG | |
| | ATACTTTATATGCAAATA | | TTATAATGAAAATGATATAAATACA | |
| | CCATATGAAGAAAAAAGT | | CCAAAAGAAATAGATGATACAACA | |
| | AAAAAATATTATAAAGCA | | AATCTTAATAATAATTATAGAAATA | |
| | TTTGAAATAATGAGAAAT | | ATCTTGATGAAGTAATACTTGATTA | |
| | GTATGGATAATACCAGAA | | TAATAGTCAAACAATACCACAAATA | |
| | AGAAATACAATAGGAACA | | AGTAATAGAACACTTAATACACTTG | |
| | AATCCAAGTGATTTTGAT | | TACAAGATAATAGTTATGTACCAAG | |
| | CCACCAGCAAGTCTTAAA | | ATATGATAGTAATGGAACAAGTGA | |
| | AATGGAAGTAGTGCATAT | | AATAGAAGAATATGATGTAGTAGA | |
| | TATGATCCAAATTATCTT | | TTTTAATGTATTTTTTTATCTTCATG | |
| | ACAACAGATGCAGAAAA | | CACAAAAAGTACCAGAAGGAGAAA | |
| | AGATAGATATCTTAAAAC | | CAAATATAAGTCTTACAAGTAGTAT | |
| | AACAATAAAACTTTTTAA | | AGATACAGCACTTCTTGAAGAAAG | |
| | AAGAATAAATAGTAATCC | | TAAAGATATATTTTTTAGTAGTGAA | |
| | AGCAGGAAAAGTACTTCT | | TTTATAGATACAATAAATAAACCAG | |
| | TCAAGAAATAAGTTATGC | | TAAATGCAGCACTTTTTATAGATTG | |
| | AAAACCATATCTTGGAAA | | GATAAGTAAAGTAATAAGAGATTTT | |
| | TGATCATACACCAATAGA | | ACAACAGAAGCAACACAAAAAAGT | |
| | TGAATTTAGTCCAGTAAC | | ACAGTAGATAAAATAGCAGATATA | |
| | AAGAACAACAAGTGTAAA | | AGTCTTATAGTACCATATGTAGGA | |
| | TATAAAACTTAGTACAAA | | CTTGCACTTAATATAATAATAGAAG | |
| | TGTAGAAAGTAGTATGCT | | CAGAAAAAGGAAATTTTGAAGAAG | |
| | TCTTAATCTTCTTGTACTT | | CATTTGAACTTCTTGGAGTAGGAA | |
| | GGAGCAGGACCAGATAT | | TACTTCTTGAATTTGTACCAGAACT | |
| | ATTTGAAAGTTGTTGTTA | | TACAATACCAGTAATACTTGTATTT | |
| | TCCAGTAAGAAAACTTAT | | ACAATAAAAAGTTATATAGATAGTT | |
| | AGATCCAGATGTAGTATA | | ATGAAAATAAAAATAAAGCAATAAA | |
| | TGATCCAAGTAATTATGG | | AGCAATAAATAATAGTCTTATAGAA | |
| | ATTTGGAAGTATAAATAT | | AGAGAAGCAAAATGGAAAGAAATA | |
| | AGTAACATTTAGTCCAGA | | TATAGTTGGATAGTAAGTAATTGG | |
| | ATATGAATATACATTTAAT | | CTTACAAGAATAAATACACAATTTA | |
| | GATATAAGTGGAGGACA | | ATAAAAGAAAAGAACAAATGTATCA | |
| | TAATAGTAGTACAGAAAG | | AGCACTTCAAAATCAAGTAGATGC | |
| | TTTTATAGCAGATCCAGC | | AATAAAAACAGCAATAGAATATAAA | |
| | AATAAGTCTTGCACATGA | | TATAATAATTATACAAGTGATGAAA | |
| | ACTTATACATGCACTTCA | | AAAATAGACTTGAAAGTGAATATAA | |
| | TGGACTTTATGGAGCAA | | TATAAATAATATAGAAGAAGAACTT | |
| | GAGGAGTAACATATGAA | | AATAAAAAAGTAAGTCTTGCAATGA | |
| | GAAACAATAGAAGTAAAA | | AAAATATAGAAAGATTTATGACAGA | |
| | CAAGCACCACTTATGATA | | AAGTAGTATAAGTTATCTTATGAAA | |
| | GCAGAAAAACCAATAAG | | CTTATAAATGAAGCAAAAGTAGGA | |
| | ACTTGAAGAATTTCTTAC | | AAACTTAAAAAATATGATAATCATG | |
| | ATTTGGAGGACAAGATCT | | TAAAAAGTGATCTTCTTAATTATAT | |
| | TAATATAATAACAAGTGC | | ACTTGATCATAGAAGTATACTTGG | |
| | AATGAAAGAAAAAATATA | | AGAACAAACAAATGAACTTAGTGA | |
| | TAATAATCTTCTTGCAAA | | TCTTGTAACAAGTACACTTAATAGT | |
| | TTATGAAAAAATAGCAAC | | AGTATACCATTTGAACTTAGTAGTT | |
| | AAGACTTAGTGAAGTAAA | | ATACAAATGATAAAATACTTATAAT | |
| | TAGTGCACCACCAGAAT | | ATATTTTAATAGACTTTATAAAAAA | |
| | ATGATATAAATGAATATA | | ATAAAAGATAGTAGTATACTTGATA | |
| | AAGATTATTTTCAATGGA | | TGAGATATGAAAATAATAAATTTAT | |
| | AATATGGACTTGATAAAA | | AGATATAAGTGGATATGGAAGTAA | |
| | ATGCAGATGGAAGTTATA | | TATAAGTATAAATGGAAATGTATAT | |
| | CAGTAAATGAAAATAAAT | | ATATATAGTACAAATAGAAATCAAT | |
| | TTAATGAAATATATAAAA | | TTGGAATATATAATAGTAGACTTAG | |
| | AACTTTATAGTTTTACAG | | TGAAGTAAATATAGCACAAAATAAT | |
| | AAAGTGATCTTGCAAATA | | GATATAATATATAATAGTAGATATC | |
| | AATTTAAAGTAAAATGTA | | AAAATTTTAGTATAAGTTTTTGGGT | |
| | GAAATACATATTTTATAA | | AAGAATACCAAAACATTATAAACCA | |
| | AATATGAATTTCTTAAAG | | ATGAATCATAATAGAGAATATACAA | |
| | TACCAAATCTTCTTGATG | | TAATAAATTGTATGGGAAATAATAA | |
| | ATGATATATATACAGTAA | | TAGTGGATGGAAAATAAGTCTTAG | |
| | GTGAAGGATTTAATATAG | | AACAGTAAGAGATTGTGAAATAAT | |
| | GAAATCTTGCAGTAAATA | | ATGGACACTTCAAGATACAAGTGG | |
| | ATAGAGGACAAAGTATAA | | AAATAAAGAAAATCTTATATTTAGA | |
| | AACTTAATCCAAAAATAA | | TATGAAGAACTTAATAGAATAAGTA | |
| | TAGATAGTATACCAGATA | | ATTATATAAATAAATGGATATTTGT | |
| | AAGGACTTGTAGAAAAAA | | AACAATAACAAATAATAGACTTGGA | |
| | TAGTAAAATTTTGTAAAA | | AATAGTAGAATATATATAAATGGAA | |
| | GTGTAATACCAAGAAAA | | ATCTTATAGTAGAAAAAAGTATAAG | |
| | | | TAATCTTGGAGATATACATGTAAGT | |
| | | | GATAATATACTTTTTAAAATAGTAG | |
| | | | GATGTGATGATGAAACATATGTAG | |
| | | | GAATAAGATATTTTAAAGTATTTAA | |
| | | | TACAGAACTTGATAAAACAGAAATA | |
| | | | GAAACACTTTATAGTAATGAACCA | |
| | | | GATCCAAGTATACTTAAAAATTATT | |
| | | | GGGGAAATTATCTTCTTTATAATAA | |
| | | | AAAATATTATCTTTTTAATCTTCTTA | |
| | | | GAAAAGATAAATATATAACACTTAA | |
| | | | TAGTGGAATACTTAATATAAATCAA | |
| | | | CAAAGAGGAGTAACAGAAGGAAGT | |
| | | | GTATTTCTTAATTATAAACTTTATG | |
| | | | AAGGAGTAGAAGTAATAATAAGAA | |
| | | | AAAATGGACCAATAGATATAAGTA | |
| | | | ATACAGATAATTTTGTAAGAAAAAA | |
| | | | TGATCTTGCATATATAAATGTAGTA | |
| | | | GATAGAGGAGTAGAATATAGACTT | |
| | | | TATGCAGATACAAAAAGTGAAAAA | |
| | | | GAAAAAATAATAAGAACAAGTAATC | |
| | | | TTAATGATAGTCTTGGACAAATAAT | |
| | | | AGTAATGGATAGTATAGGAAATAA | |
| | | | TTGTACAATGAATTTTCAAAATAAT | |
| | | | AATGGAAGTAATATAGGACTTCTT | |
| | | | GGATTTCATAGTAATAATCTTGTAG | |
| | | | CAAGTAGTTGGTATTATAATAATAT | |
| | | | AAGAAGAAATACAAGTAGTAATGG | |
| | | | ATGTTTTTGGAGTAGTATAAGTAAA | |
| | | | GAAAATGGATGGAAAGAA | |
| BONT/G | CCAGTAAATATAAAANNN | 41 | AATACAGGAAAAAGTGAACAATGT | 42 |
| Q60393 | TTTAATTATAATGATCCA | | ATAATAGTAAATAATGAAGATCTTT | |
| | ATAAATAATGATGATATA | | TTTTTATAGCAAATAAAGATAGTTT | |
| | ATAATGATGGAACCATTT | | TAGTAAAGATCTTGCAAAAGCAGA | |
| | AATGATCCAGGACCAGG | | AACAATAGCATATAATACACAAAAT | |
| | AACATATTATAAAGCATT | | AATACAATAGAAAATAATTTTAGTA | |
| | TAGAATAATAGATAGAAT | | TAGATCAACTTATACTTGATAATGA | |
| | ATGGATAGTACCAGAAA | | TCTTAGTAGTGGAATAGATCTTCC | |
| | GATTTACATATGGATTTC | | AAATGAAAATACAGAACCATTTACA | |
| | AACCAGATCAATTTAATG | | AATTTTGATGATATAGATATACCAG | |
| | CAAGTACAGGAGTATTTA | | TATATATAAAACAAAGTGCACTTAA | |
| | GTAAAGATGTATATGAAT | | AAAAATATTTGTAGATGGAGATAGT | |
| | ATTATGATCCAACATATC | | CTTTTTGAATATCTTCATGCACAAA | |
| | TTAAAACAGATGCAGAAA | | CATTTCCAAGTAATATAGAAAATCT | |
| | AAGATAAATTTCTTAAAA | | TCAACTTACAAATAGTCTTAATGAT | |
| | CAATGATAAAACTTTTTA | | GCACTTAGAAATAATAATAAAGTAT | |
| | ATAGAATAAATAGTAAAC | | ATACATTTTTTAGTACAAATCTTGT | |
| | CAAGTGGACAAAGACTT | | AGAAAAAGCAAATACAGTAGTAGG | |
| | CTTGATATGATAGTAGAT | | AGCAAGTCTTTTTGTAAATTGGGTA | |
| | GCAATACCATATCTTGGA | | AAAGGAGTAATAGATGATTTTACAA | |
| | AATGCAAGTACACCACC | | GTGAAAGTACACAAAAAAGTACAA | |
| | AGATAAATTTGCAGCAAA | | TAGATAAAGTAAGTGATGTAAGTAT | |
| | TGTAGCAAATGTAAGTAT | | AATAATACCATATATAGGACCAGC | |
| | AAATAAAAAAATAATACA | | ACTTAATGTAGGAAATGAAACAGC | |
| | ACCAGGAGCAGAAGATC | | AAAAGAAAATTTTAAAAATGCATTT | |
| | AAATAAAAGGACTTATGA | | GAAATAGGAGGAGCAGCAATACTT | |
| | CAAATCTTATAATATTTG | | ATGGAATTTATACCAGAACTTATAG | |
| | GACCAGGACCAGTACTT | | TACCAATAGTAGGATTTTTTACACT | |
| | AGTGATAATTTTACAGAT | | TGAAAGTTATGTAGGAAATAAAGG | |
| | AGTATGATAATGAATGGA | | ACATATAATAATGACAATAAGTAAT | |
| | CATAGTCCAATAAGTGAA | | GCACTTAAAAAAAGAGATCAAAAA | |
| | GGATTTGGAGCAAGAAT | | TGGACAGATATGTATGGACTTATA | |
| | GATGATAAGATTTTGTCC | | GTAAGTCAATGGCTTAGTACAGTA | |
| | AAGTTGTCTTAATGTATT | | AATACACAATTTTATACAATAAAAG | |
| | TAATAATGTACAAGAAAA | | AAAGAATGTATAATGCACTTAATAA | |
| | TAAAGATACAAGTATATT | | TCAAAGTCAAGCAATAGAAAAAAT | |
| | TAGTAGAAGAGCATATTT | | AATAGAAGATCAATATAATAGATAT | |
| | TGCAGATCCAGCACTTA | | AGTGAAGAAGATAAAATGAATATA | |
| | CACTTATGCATGAACTTA | | AATATAGATTTTAATGATATAGATT | |
| | TACATGTACTTCATGGAC | | TTAAACTTAATCAAAGTATAAATCT | |
| | TTTATGGAATAAAAATAA | | TGCAATAAATAATATAGATGATTTT | |
| | GTAATCTTCCAATAACAC | | ATAAATCAATGTAGTATAAGTTATC | |
| | CAAATACAAAAGAATTTT | | TTATGAATAGAATGATACCACTTGC | |
| | TTATGCAACATAGTGATC | | AGTAAAAAAACTTAAAGATTTTGAT | |
| | CAGTACAAGCAGAAGAA | | GATAATCTTAAAAGAGATCTTCTTG | |
| | CTTTATACATTTGGAGGA | | AATATATAGATACAAATGAACTTTA | |
| | CATGATCCAAGTGTAATA | | TCTTCTTGATGAAGTAAATATACTT | |
| | AGTCCAAGTACAGATATG | | AAAAGTAAAGTAAATAGACATCTTA | |
| | AATATATATAATAAAGCA | | AAGATAGTATACCATTTGATCTTAG | |
| | CTTCAAAATTTTCAAGAT | | TCTTTATACAAAAGATACAATACTT | |
| | ATAGCAAATAGACTTAAT | | ATACAAGTATTTAATAATTATATAA | |
| | ATAGTAAGTAGTGCACAA | | GTAATATAAGTAGTAATGCAATACT | |
| | GGAAGTGGAATAGATAT | | TAGTCTTAGTTATAGAGGAGGAAG | |
| | AAGTCTTTATAAACAAAT | | ACTTATAGATAGTAGTGGATATGG | |
| | ATATAAAAATAAATATGA | | AGCAACAATGAATGTAGGAAGTGA | |
| | TTTTGTAGAAGATCCAAA | | TGTAATATTTAATGATATAGGAAAT | |
| | TGGAAAATATAGTGTAGA | | GGACAATTTAAACTTAATAATAGTG | |
| | TAAAGATAAATTTGATAA | | AAAATAGTAATATAACAGCACATCA | |
| | ACTTTATAAAGCACTTAT | | AAGTAAATTTGTAGTATATGATAGT | |
| | GTTTGGATTTACAGAAAC | | ATGTTTGATAATTTTAGTATAAATTT | |
| | AAATCTTGCAGGAGAATA | | TTGGGTAAGAACACCAAAATATAA | |
| | TGGAATAAAAACAAGATA | | TAATAATGATATACAAACATATCTT | |
| | TAGTTATTTTAGTGAATA | | CAAAATGAATATACAATAATAAGTT | |
| | TCTTCCACCAATAAAAAC | | GTATAAAAAATGATAGTGGATGGA | |
| | AGAAAAACTTCTTGATAA | | AAGTAAGTATAAAAGGAAATAGAA | |
| | TACAATATATACACAAAA | | TAATATGGACACTTATAGATGTAAA | |
| | TGAAGGATTTAATATAGC | | TGCAAAAAGTAAAAGTATATTTTTT | |
| | AAGTAAAAATCTTAAAAC | | GAATATAGTATAAAAGATAATATAA | |
| | AGAATTTAATGGACAAAA | | GTGATTATATAAATAAATGGTTTAG | |
| | TAAAGCAGTAAATAAAGA | | TATAACAATAACAAATGATAGACTT | |
| | AGCATATGAAGAAATAAG | | GGAAATGCAAATATATATATAAATG | |
| | TCTTGAACATCTTGTAAT | | GAAGTCTTAAAAAAAGTGAAAAAAT | |
| | ATATAGAATAGCAATGTG | | ACTTAATCTTGATAGAATAAATAGT | |
| | TAAACCAGTAATGTATAA | | AGTAATGATATAGATTTTAAACTTA | |
| | A | | TAAATTGTACAGATACAACAAAATT | |
| | | | TGTATGGATAAAAGATTTTAATATA | |
| | | | TTTGGAAGAGAACTTAATGCAACA | |
| | | | GAAGTAAGTAGTCTTTATTGGATA | |
| | | | CAAAGTAGTACAAATACACTTAAA | |
| | | | GATTTTTGGGGAAATCCACTTAGA | |
| | | | TATGATACACAATATTATCTTTTTA | |
| | | | ATCAAGGAATGCAAAATATATATAT | |
| | | | AAAATATTTTAGTAAAGCAAGTATG | |
| | | | GGAGAAACAGCACCAAGAACAAAT | |
| | | | TTTAATAATGCAGCAATAAATTATC | |
| | | | AAAATCTTTATCTTGGACTTAGATT | |
| | | | TATAATAAAAAAAGCAAGTAATAGT | |
| | | | AGAAATATAAATAATGATAATATAG | |
| | | | TAAGAGAAGGAGATTATATATATCT | |
| | | | TAATATAGATAATATAAGTGATGAA | |
| | | | AGTTATAGAGTATATGTACTTGTAA | |
| | | | ATAGTAAAGAAATACAAACACAACT | |
| | | | TTTTCTTGCACCAATAAATGATGAT | |
| | | | CCAACATTTTATGATGTACTTCAAA | |
| | | | TAAAAAAATATTATGAAAAAACAAC | |
| | | | ATATAATTGTCAAATACTTTGTGAA | |
| | | | AAAGATACAAAAACATTTGGACTTT | |
| | | | TTGGAATAGGAAAATTTGTAAAAG | |
| | | | ATTATGGATATGTATGGGATACATA | |
| | | | TGATAATTATTTTTGTATAAGTCAA | |
| | | | TGGTATCTTAGAAGAATAAGTGAA | |
| | | | AATATAAATAAACTTAGACTTGGAT | |
| | | | GTAATTGGCAATTTATACCAGTAG | |
| | | | ATGAAGGATGGACAGAA | |

Any combination of light chain and heavy chain may be expressed in a cell to make a di-chain BoNT. In some embodiments, a light chain and a heavy chain of the same serotype are expressed in a cell to form a di-chain BoNT. For example, a light chain serotype A and a heavy chain serotype A are expressed in a cell to form a di-chain BoNT. In some embodiments, a light chain and a heavy chain of different serotype are expressed in a cell to form a di-chain BoNT. For example, a light chain serotype A and a heavy chain serotype E are expressed in a cell to form a di-chain BoNT.

In some embodiments, the di-chain BoNT formed is active. For example, an active light chain serotype A and a heavy chain serotype A may be expressed in a cell to produce an active di-chain BoNT. In some embodiments, the di-chain BoNT formed is inactive. For example, an inactive light chain serotype A and a heavy chain serotype A may be expressed in a cell to produce a di-chain iBoNT.

In some embodiments, the ratio of nucleic acid sequence encoding a light chain to nucleic acid sequence encoding a heavy chain expressed in a cell is 1:1. In some embodiments, the ratio of nucleic acid sequence encoding a light chain to nucleic acid sequence encoding a heavy chain expressed in a cell is 2:1. In some embodiments, the ratio of nucleic acid sequence encoding a light chain to nucleic acid sequence encoding a heavy chain expressed in a cell is 3:1. In some embodiments, the ratio of nucleic acid sequence encoding a light chain to nucleic acid sequence encoding a heavy chain expressed in a cell is 4:1. In some embodiments, the ratio of nucleic acid sequence encoding a light chain to nucleic acid sequence encoding a heavy chain expressed in a cell is 1:2. In some embodiments, the ratio of nucleic acid sequence encoding a light chain to nucleic acid sequence encoding a heavy chain expressed in a cell is 1:3. In some embodiments, the ratio of nucleic acid sequence encoding a light chain to nucleic acid sequence encoding a heavy chain expressed in a cell is 1:4.

The di-chain BoNT made in accordance with the present invention may also be glycosylated when the light chain and the heavy chain are expressed in a host cell that has the biological machinery to glycosylate the expressed toxin. Hereinafter, a glycosylated BoNT is referred to as g-BoNT. In some embodiments, the host cell is capable of glycosylating the expressed toxin with at least one of an N-acetylglucosamine, mannose, glucose, galactose, fructose, sialic acid and/or an oligosaccharide comprising two or more of the identified saccharides. In some embodiments, eukaryotic systems may be used to produce g-BoNT, or fragments thereof. For example, yeast may be used to express large amounts of glycoprotein at low cost. However, a major draw back of using yeast is that both N- and O-glycosylation apparatus differs from that of higher eukaryotes. In some embodiments, mammalian cells are used as host for expression genes obtained from higher eukaryotes because the signal for synthesis, processing and secretion of these proteins are usually recognized by the cells. For example, Chinese Hamster Ovary (CHO) cells are very well known for production of eukaryotic proteins or glycoproteins, since these cells can grow either attached to the surface or in suspension and adapt well to growth in the absence of serum. Researchers have developed several CHO mutant cell lines carrying one or more glycosylation mutation/s. Stanley, P., Molecular and Cellular Biology, 9(2):377-383 (1989). These mutant cell lines are called “Lec” for Lectin resistant. Stanley, P. et al., Cell, 6: 121-128 (1975). These cell lines lack one or more of the key enzymes involved in the glycosylation pathway, thus resulting in the production of glycoprotein with carbohydrates of defined structure and minimal heterogeneity. Lec-1 is one such cell line which lacks a key enzyme N-acetyl Glucosaminetransferase-1. The absence of this enzyme results in the inhibition of glycosylation pathway after the carbohydrates trim down to Man(2)GlcNAc(2), leading to production of reduced, but homogeneous glycosylation (Man=manose and GlcNAc=n-acetylglucosamine).

In some embodiments, the light chain and heavy chain of the present invention are expressed in insect cells, so that the resulting di-chain BoNT is glycosylated. For example, baculovirus based expression system makes insect cell lines an ideal system for high-level transient expression of glycoproteins. Proteins that are N-glycosylated in vertebrate cells are also generally glycosylated in insect cells. The first step of N-glycosylation in insect cells is similar to that in vertebrates. Usually, the Man(9)GlcNaC(2) moiety is trimmed to shorter oligosaccharide structures of Man(3)GlcNAc(2) in both insect cells and vertebrates. In vertebrates, these shorter core structures serve as the framework for complex oligosaccharide synthesis, while in insect cells this additional, complex oligosaccharide synthesis does not appear to occur in many cases, thus leading to restricted and less heterogeneous glycosylation.

Sometimes the natural glycosylation system in insect cells may not meet the requirement of the complex glycosylation for protein therapeutics. In such a case, a special cell line may be used, such as Mimic Sf9 insect cell (available from Invitrogen, Carlsbad, Calif., USA) for high level expression of complex glycoproteins in insect cells. Hollister, J. et al., Biochemistry, 41:15093-15104 (2002); Hollister, J. et al., Glycobiology 11:1-9 (2001); Hollister, J. et al., Glycobiology, 8:473-480 (1998); Jarvis, D. et al., Curr Opin Biotechnol, 9:528-533 (1998); and Seo, N. S. et al., Protein Expr Purif, 22: 234-241. Briefly, mammalian cells require expensive media supplements and expression levels are relatively low when compared to expression in other hosts. Insect cells offer several advantages over mammalian cells—growth at room temperature, lower media costs, and production of high levels of recombinant protein. The disadvantage of using insect cells is that the majority of proteins produced do not exhibit the complex glycosylation seen in mammlian cells. This can affect protein function, structure, antigeniticity and stabililty. The Mimic Sf9 Insect Cell Line contains stably integrated mammalian glycosyltransferases, resulting in the production of biantennary N-glycans. Mimic Sf9 Insect Cells enable expression of proteins that are similar to what would be produced in mammalian cells, making them suitable for producing proteins to of the present invention.

In some embodiments, the di-chain BoNTs are glycosylated at one or more N-glycosylation sites. For example, an N-glycosylation site include the consensus pattern Asn-Xaa-Ser/Thr. It is noted, however, that the presence of the consensus tripeptide is not sufficient to conclude that an asparagine residue is glycosylated, due to the fact that the folding of the protein plays an important role in the regulation of N-glycosylation. It has been shown that the presence of proline between Asn and Ser/Thr will inhibit N-glycosylation.

In some embodiments, the g-BoNT is glycosylated at one or more O-glycosylation sites. O-glycosylation sites are usually found in helical segments which means they are uncommon in the beta-sheet structure. Currently, there is no known consensus pattern for an O-glycosylation site.

Crystal structure of BoNT/A-Allergan shows the potential sites of N-glycosylation on the surface as follows: 173-NLTR (SEQ ID NO: 3), 382-NYTI (SEQ ID NO: 4), 411-NFTK (SEQ ID NO: 5), 417-NFTG (SEQ ID NO: 6), 971-NNSG (SEQ ID NO: 7), 1010-NISD (SEQ ID NO: 8), 1198-NASQ (SEQ ID NO: 9), 1221-NLSQ (SEQ ID NO: 10). In some embodiments, g-BoNT/A (including g-iBoNT/A) is glycosylated at 173-NLTR (SEQ ID NO: 11), 382-NYTI (SEQ ID NO: 12), 411-NFTK (SEQ ID NO: 13), 417-NFTG (SEQ ID NO: 14), 971-NNSG (SEQ ID NO: 15), 1010-NISD (SEQ ID NO: 16), 1198-NASQ (SEQ ID NO: 17) and/or 1221-NLSQ (SEQ ID NO: 18). Potential sites of N-glycosylation for BoNT/E are as follows: 97-NLSG (SEQ ID NO: 19), 138-NGSG (SEQ ID NO: 20), 161-NSSN (SEQ ID NO: 21), 164-NISL (SEQ ID NO: 22), 365-NDSI (SEQ ID NO: 23), and 370-NISE. In some embodiments, g-BoNT/E (including g-iBoNT/E) is glycosylated at 97-NLSG, 138-NGSG, 161-NSSN, 164-NISL, 365-NDSI, and/or 370-NISE (SEQ ID NO: 24).

In some embodiments, BEVS-insect cells may glycosylate a protein in endoplasmic reticulum (ER) on its consensus Asn-X-Ser/Thr recognized in an appropriate context by oligosaccharyltransferase found in the ER and Golgi complex.

Like most eukaryotic ERs, insect ER enzymes can attach at least a Glc₃Man_{g}GlcNAc₂ (molecular weight of about 2600 dalton). The Glc₃Man_{g}GlcNAc₂ is the core structure that serves as the framework for complex oligosaccharide synthesis involving further GlcNAc, Gal or sialic-acid additions.

In some embodiments, a g-BoNT (including g-iBoNT) of the present invention comprises more than one Glc₃Man_{g}GlcNAc₂, for example five to twenty Glc₃Man_{g}GlcNAc₂. In some embodiments, the glycosylation constitute more than about 2% of the g-BoNT (including g-iBoNT) by weight. In some embodiments, the glycosylation constitute more than about 5% of the g-BoNT (including g-iBoNT) by weight. In some embodiments, the glycosylation constitute more than about 10% of the g-BoNT (including g-iBoNT) by weight.

In some embodiments, the g-BoNT/A or g-iBoNT/A is about 150 kDa, and the glycosylation adds about 20 to 30 kDa to the protein. In some embodiments, the g-BoNT/A or the g-iBoNT/A has about eight to twelve Glc₃Man_{g}GlcNAc₂ (molecular weight of about 2600 dalton). In some embodiments, the g-BoNT/A or g-iBoNT/A is glycosylated with Glc₃Man_{g}GlcNAc₂ at positions 173-NLTR, 382-NYTI, 411-NFTK, 417-NFTG, 971-NNSG, 1010-NISD, 1198-NASQ, 1221-NLSQ.

Di-chain BoNTs produced in accordance with the present invention may be used to treat various conditions. For example, the di-chain BoNT may be used to treat muscular disorder, autonomic nervous system disorder and pain. Non-limiting examples of neuromuscular disorders that may be treated with a modified neurotoxin include strabismus, blepharospasm, spasmodic torticollis (cervical dystonia), oromandibular dystonia and spasmodic dysphonia (largyngeal dystonia). Non-limiting examples of autonomic nervous system disorders include rhinorrhea, otitis media, excessive salivation, asthma, chronic obstructive pulmonary disease (COPD), excessive stomach acid secretion, spastic colitis and excessive sweating. Non-limiting examples of pain which may be treated in accordance to the present invention include migraine headache pain that is associated with muscle spasm, vascular disturbances, neuralgia, neuropathy and pain associated with inflammation.

An ordinarily skilled medical provider can determine the appropriate dose and frequency of administration(s) to achieve an optimum clinical result. Also, the appropriate route of administration and dosage are generally determined on a case by case basis by the attending physician. Such determinations are routine to one of ordinary skill in the art (see for example, Harrison's Principles of Internal Medicine (1998), edited by Anthony Fauci et al., 14^{th} edition, published by McGraw Hill).

The present invention also includes formulations which comprise at least one of the compositions disclosed herein, e.g, di-chain BoNT, di-chain iBoNT, NTNH, active g-BoNT, g-iBoNT, etc. In some embodiments, the formulations comprise at least one of a di-chain BoNT produced in accordance with the present invention in a pharmacologically acceptable carrier, such as sterile physiological saline, sterile saline with 0.1% gelatin, or sterile saline with 1.0 mg/ml bovine serum albumin.

In order that the invention disclosed herein may be more efficiently understood, examples are provided below. It should be understood that these examples are for illustrative purposes only and are not to be construed as limiting the invention in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1

### Co-Expression of BoNT-LC and BoNT-HC in Insect Cells with Baculovirus Expression System

Eukaryotic expression systems employing insect cell hosts may be based upon either plasmid vectors or plasmid-virion hybrid vectors. Examples of insect hosts include the common fruit fly, Drosophila melanogaster, the mosquito (Aedes albopictus), the fall army worm (Spodoptera frugiperda), the cabbage looper (Trichoplusia ni), the salt marsh caterpillar (Estigmene acrea) or the silkworm (Bombyx mori). Heterologous protein overexpression is often in suspension cell cultures, however, one of the advantages of plasmid-virion systems is that the recombinant virus may also be injected into larval host hemocel or even fed to the mature host.

Plasmid-based vector systems provide a mechanism for both transient and long-term expression of recombinant protein. This expression system is exemplified by the Drosophila Expression System (DES) available from Invitrogen (Carlsbad, Calif.). The transfection of competent D. melanogaster cells with engineered plasmid will mediate the transient (2-7 days) expression of heterologous protein. Establishing transformed cells for longer term expression of protein requires that the host cells be cotransfected with a “selection” vector, which results in the stable integration of the expression cassette into the host genome. The DES system offers means for either constitutive or inducible expression. Constitutive expression is mediated using the Ac5 Drosophila promoter, whereas copper-inducible expression is driven by the metallothionein promoter. The DES vectors are designed with multiple cloning sites for insertion of the heterologous protein gene in any of three reading frames, and a choice of vectors provides for the expression of a variety of C-terminal fusion tags: V5 epitope for identification of expressed protein with V5 epitope antibody, polyhistidine peptide for simplified purification with metal chelate affinity resin, and the BiP secretion leader peptide.

In some embodiments, the plasmid-virion system is based upon the large, double stranded DNA baculovirus. The Autographica californica (alfalfa looper) nuclear polyhedrosis virus (AcNPV) virion is the most common source of the “expression cassette” for this system. Another source is the Bombyx mori (silkworm) NPV virion (BmNPV). One advantage of the baculovirus-insect expression system is the large native size of the viral genome. In the expression cassettes, many elements of the native genome unnecessary for viral replication and production are removed, allowing the insertion of a large heterologous gene or several genes (each under its own promoter in a multipromoter cassette) encoding the protein of interest for expression. Thus, the plasmid-virion system enables the expression of large proteins and/or the various protein components of large hetero-oligomeric complexes. Additionally, the virion has a broad host range, so any of a number of established insect cell lines can be used for overproduction of recombinant protein or inject larval host hemocel for in situ studies.

The baculovirus expression cassette contains all the genetic information needed for propagation of progeny virus, so no helper virus is needed in the transfection process. The biology of the virus provides a simple means, using plaque morphology, to identify transformed host cells. Heterologous protein genes are under the control of the late-stage baculovirus p10 and polyhedrin promoters, and recombinant protein is, in most cases, the sole product produced. Cells harboring the baculovirus expression cassette integrated in their genomes thereby produce relatively high amounts of heterologous protein, and most of this protein is easily extracted from the cytoplasm or harvested from extracellular culture filtrate (when the expression cassette includes a secretory leader fusion peptide engineered to the recombinant protein). Additionally, some viral vectors are fitted with hybrid early/late promoters that permit the processing of glycosylated or secreted proteins.

The process of creating and expressing heterologous protein begins with the engineering of the heterologous protein gene into a “transfer plasmid.” This plasmid vector may contain all the elements for autonomous replication in Escherichia coli, a bacterial selection marker (an ampicillin resistance gene, for example), and elements of the baculovirus genome. The heterologous protein gene is inserted in a specific orientation and location into the plasmid so it is flanked by elements of the baculovirus genome. Successfully engineered plasmids are then cotransfected with viral expression vector (essentially wild-type baculovirus DNA with p10 and/or polyhedrin genes removed) into permissive host cells. Cell-mediated double recombination between viral sequences flanking the heterologous protein gene and the corresponding sequences of the viral expression vector results in the incorporation of the heterologous protein gene into the viral genome. Hence, recombinant progeny viruses will produce heterologous protein late in their life cycle.

Over 30 different transfer vectors and 3 different baculovirus expression vectors are available from Novagen (EMD Biosciences Inc., Novagen Brand, Madison, Wis.). Many baculovirus expression vectors have a deleted polyhedron gene, with only the promoter remaining for driving expression of the protein of interest, but the BacVector-2000 lacks polyhedron and several additional non-essential genes. The BacVector-3000 is similar to the BacVector-2000, but further lacks protease and chitinase genes that reduce degradation of expressed proteins and decrease cell lysis. Transfer vectors from Novagen allow positive screening with the gus reporter gene, as well as N- and C-terminal peptide tags (cellulose binding domain, polyhistidine, and S-Tag™) to facilitate identification and purification, and secretory leader peptide (gp64) to direct extracellular export of the expressed protein product. There is also a choice of early, early/late, or very late (polyhedrin, p10, or pg64) promoters in the transfer vectors.

pBAC™-1, pBAC4x-1 and pBACgus-1 are baculovirus transfer plasmid vectors designed for simplified cloning and expression of target genes in insect cells. For example, the multipromoter transfer vector, pBAC4x-1, allows the engineering of up to four target genes under the control of separate promoters (two polyhedrin and two p10, each of which is upstream of unique cloning sites for sequential insertion of target genes, and the homologous promoters are in opposite orientations to minimize recombination), enabling expression of up to four different proteins simultaneously in insect cells. For virus surface display, Novagen's pBACsurf-1 incorporates a gp64 secretory signal peptide and anchoring sequences in fusions. The cloning of PCR products directly into transfer vectors is also possible with ligation-independent cloning-competent pBAC2, 7, and 8 vectors.

For co-expression of BoNT-LC and BoNT-HC using the baculoviral system, BoNT-LC and BoNT-HC may be subcloned into the pBAC4x-1 transfer plasmid. The pBAC4x-1 transfer vector contains a large tract of AcNPV sequence flanking the subcloning region to facilitate homologous recombination. Co-transfection of the transfer recombinant plasmid and Autographa californica nuclear polyhedrosis virus (AcNPV) DNA into insect Sf9 cells allows recombination between homologous sites, transferring the heterologous gene from the transfer plasmid to the AcNPV DNA. AcNPV infection of Sf9 cells results in the shut-off of host gene expression allowing for a high rate of recombinant mRNA and protein production. Thus, after the cell-mediated double recombination between viral sequences and the corresponding sequences of transfer vector results in the incorporation of the heterologous protein gene into the viral genome, the BoNT-LC and BoNT-HC genes will each be under control of its own promoter, and recombinant progeny baculoviruses will co-express, separately, both the BoNT-LC and BoNT-HC proteins in the same transfected insect cells.

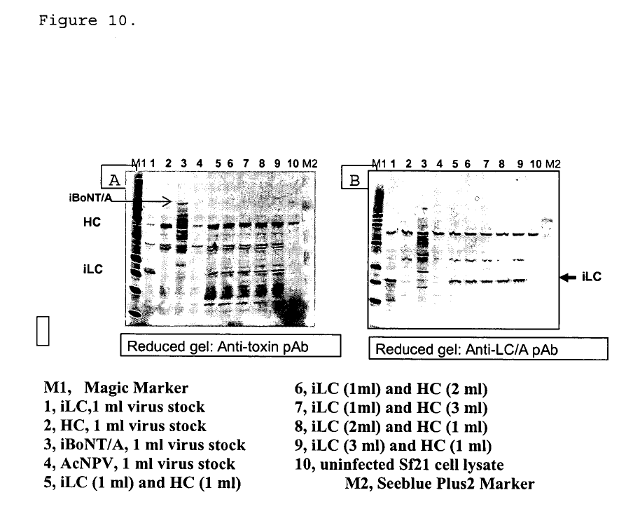

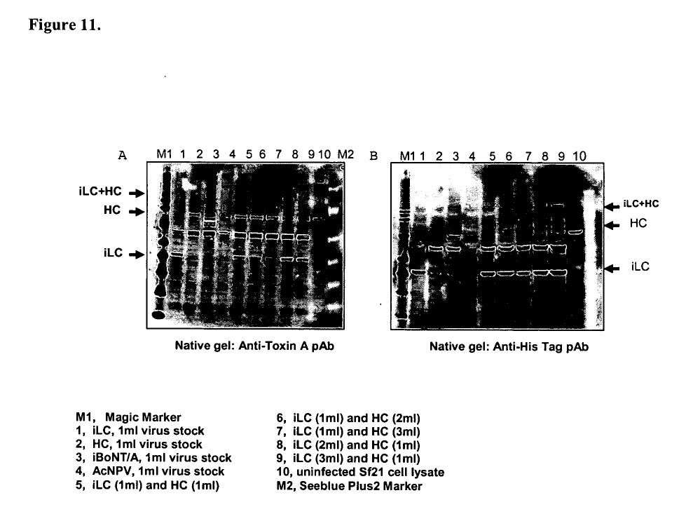

FIGS. 10 and 11 show data that BEVS has the capacity of di-chain formation of iBoNT/A in co-infection of iLC and HC recombinant baculovirus.

### Example 2

### Co-Expression of BoNT-LC and BoNT-HC in Yeast Cells

Yeast hosts that can be used for heterologous protein expression include Saccharomyces cerevisiae, Schizosaccharomyces pombe, Pichia pastoris, Hansela polymorpha, Kluyveromyces lactis, and Yarrowia lipolytica. A multitude of strains and an extensive knowledge base on the genetics and life cycle of unicellular yeasts are readily available, and several methods of transformation, including lithium acetate and electroporation-mediated transformation of intact yeast cells, are known to those of ordinary skill in the art. Yeasts are attractive as expression hosts for a number of reasons. They can be rapidly grown on minimal (inexpensive) media. Recombinants can be easily selected by complementation, using any one of a number of selectable markers. Expressed proteins can be specifically engineered for cytoplasmic localization or for extracellular export. Also, yeasts are well-suited for large scale fermentation to produce large quantities of heterologous protein. P. pastoris. K. lactis and Y. lipolytica have been extensively utilized in the industrial-scale production of metabolites and native proteins (for example, β-galactosidase). The methylotrophic yeasts, H. polymorpha and P. pastoris, both of which can grow using methanol as the sole carbon source, provide another host alternative for many researchers. P. pastoris has produced some of the highest heterologous protein yields to date (12 g/L fermentation culture), in some cases 10 to 100-fold greater than yields from S. cerevisiae. In P. pastoris, growth in methanol is mediated by alcohol oxidase, an enzyme whose de novo synthesis is tightly regulated by the alcohol oxidase promoter (AOX1). The enzyme has a very low specific activity. To compensate for its low specific activity, it is overproduced, accounting for more than 30 percent of total soluble protein in methanol-induced cells. The AOX1 promoter has been characterized and incorporated into a series of P. pastoris expression vectors. For example, one P. pastoris expression system is available from Invitrogen (Carlsbad, Calif.). By engineering a heterologous protein gene downstream of the genomic AOX1 promoter, one can induce the its overproduction and secretion in the medium. Because proteins produced in P. pastoris are typically posttranslationally modified, folded and processed (including disulfide bond formation) similarly to those in higher eukaryotes, the fermentation of genetically engineered P. pastoris provides an excellent means for expressing heterologous proteins. A number of proteins have been produced using this system, including tetanus toxin fragment, Bordatella pertussis pertactin, human serum albumin and lysozyme.

Yeast vectors for protein expression generally contain a plasmid origin of replication, an antibiotic resistance “marker” gene (to aid cloning and screening of plasmid constructs in E. coli), a constitutive or inducible promoter (to drive expression of the heterologous gene), and a termination signal, and may further include a signal sequence (encoding secretion leader peptides), and/or fusion protein genes (to facilitate purification). Vectors which can integrate into the yeast genome for stable transfection of heterologous sequences are also available.

The Easyselect™ Pichia Expression Kit (Invitrogen, Carlsbad, Calif.) includes the pPICZ series of vectors, P. pastoris strains, reagents for transformation, sequencing primers, media, and a comprehensive manual. Other vectors and strains are also widely available. For example, a his4−, arg4− P. pastoris host strain, which has defects in enzymes required for the synthesis of histidine and arginine, can be used in combination with vectors containing the his4+ and arg4+ marker genes for selection of complementation. Thus, using recombinant DNA methods standard in the art, the full-length BoNT-LC and BoNT-HC can be subcloned into the appropriate reading frame for in-frame expression, using cloning sites into the Pichia expression vectors pARG815 (complementing arg4− in the host) and pAO815 (complementing his4− in the host), respectively, and cotransformed into the host strain. Transfectants coexpressing both BoNT/A-LC and BoNT/A-HC peptides can thereby be selected based upon their ability to grow on media lacking histidine and arginine.

In some embodiments, the BoNT-LC and BoNT-HC genes can be subcloned, in tandem, into a single expression vector, with each gene under control of a separate promoter, and with 3′ transcription terminator sequences separating them from adjacent genes. Thus, the BoNT-LC and BoNT-HC gene products can be independently expressed by one vector construct in the same transfected cells.

Protein expression can be induced by growth on methanol-containing media, and cultures of clone coexpressing BoNT-LC and BoNT-HC can be harvested 60 h after induction, lysed in a buffer containing Triton X-100, centrifuged, and samples of the soluble and insoluble fractions of the cell lysates can be analysed by SDS-PAGE followed by Western blotting with an antibody to the BoNT-LC and BoNT-HC peptides to confirm their expression. Alternatively, if the vectors also encode epitope tags, well-characterized antibodies are readily available for confirmation of the expression products and/or complexes by Western blot analysis.

It will be understood by those of ordinary skill in the art that other eukaryotic expression vectors can also be employed in the present invention. In some embodiments, plant cells (for example, Arabidopsis thaliana, Zea mays, Nicotiana benthamina and Nicotiana tabacum) can be used in combination with vectors (for example, the T-DNA of Agrobacterium tumefaciens, or viruses based on the tobacco mosaic virus (TMV) or potato virus X (PVX) for expression of heterologous gene products. In some embodiments, amphibian cells (for example, Xenopus laevis oocytes or Xenopus cell-free extracts) in combination with recombinantly engineered expression vectors can be used as systems for the expression of heterologous proteins. In some embodiments, mammalian cells (for example, Chinese Hamster Ovary (CHO) cells or HEK 293 cells) can be used in combination with viral or virion-based expression systems (such as adenovirus-based expression systems) for the expression of heterologous gene products, and are thus within the scope of this invention.

### Example 3

### Expression of BoNT/A-LC in BEVS

(1) Construction of Wild-Type or Mutant BoNT/A-LC into pBAC-1 and pBACgus-1

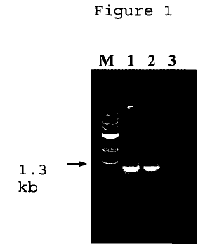

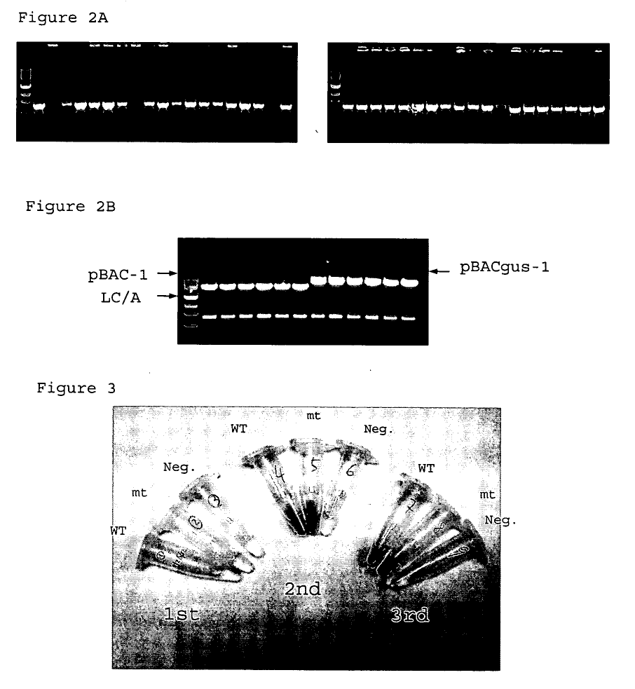

The PCR primers have been designed to amplify either wild-type BoNT/A-LC with Hall-A strain genomic DNA as template, or mutant LC H227Y with pNTP55 as template. The sense PCR primer 5′-CA GGA TCC ATG CCA TTT GTT MT AAA CAA TTT-3′ (SEQ ID NO: 25) with restriction site BamHI at 5′ end. Whereas, the antisense PCR primer 5′-CCCCCTCGAG CTTATTGTATCCTTTATCTAATGA-3′ (SEQ ID NO: 26) with XhoI restriction site at 3′ end. PCR amplified BoNT/A-LC fragment is about 1.3 kb (FIG. 1). Both wild type and mutated BoNT/A-LC inserts were cloned into pBAC-1 and pBACgus-1 transfer vectors at BamHI and XhoI cloning sites. The positive clones were selected and confirmed by PCR Screening (FIG. 2A), restriction enzymes digestion (FIG. 2B), and DNA sequencing.

(2) Co-Transfection of AcNPV with the Transfer Plasmid for Generating Recombinant Baculovirus In Vivo to Make Baculovirally-Expressed BoNT/A-LC

As described above, we have subcloned both wild type and inactive mutant BoNT/A-LC (H227Y) into a transfer vectors, pBAC-1 and pBACgus-1. Each transfer vector contains a large tract of AcNPV sequence flanking the subcloning region to facilitate homologous recombination. Co-transfection of the transfer recombinant plasmid and Autographa californica nuclear polyhedrosis virus (AcNPV) DNA into insect Sf9 cells allows recombination between homologous sites, transferring the heterologous gene from the vector to the AcNPV DNA. AcNPV infection of Sf9 cells results in the shut-off of host gene expression allowing for a high rate of recombinant mRNA and protein production.

For each transfection, 1.25×10⁶ exponentially growing Sf9 cells were seeded. The cells were allowed to attach to the plate for 20-min. During this 20-min incubation, the transfection mixture was prepared. A 500-ng of transfer plasmid LC/A gene, either wild type or mutant, 100-ng of linearized AcNPV, and 5 ul of Eufectin were respectively mixed in a sterile polystyrene tube. This DNA/Eufectin mixture was incubated at RT for 15 min. The medium instead of plasmid DNA was used as a negative control. After the DNA/Eufectin 15-min incubation was completed, 0.45 ml of room temperature medium (no antibiotics or serum) was added to the DNA/Eufectin mixture. The entire 0.5-ml of this mixture was added to the 1 ml of medium covering the cells in the plate. After 1-hour incubation at 27° C., 6 ml of medium containing 5% serum and antibiotics were added and the resultants were incubated at 27° C. for 5 days (1^{st} run). The transfection samples were listed in the Table 2 below.

TABLE 2
rLC transfection samples

| | transfer plasmids | description of insert |
|---|---|---|
| 1 | pBAC-1/BoNT/A-LC, | LC of BoNT/A, inactive mutant |
| | H227Y | H227Y (mt) |
| 2 | pBAC-1/BoNT/A-LC | LC of BoNT/A, wild type (wt) |
| 3 | pBACgus-1/BoNT/A-LC, | LC of BoNT/A, inactive mutant |
| | H227Y | H227Y (mt) |
| 4 | pBACgus-1/BoNT/A-LC | LC of BoNT/A, wild type (wt) |
| 5 | AcNPV only | Baculovirus vector alone, |
| | | negative control |

(3) Amplification of Recombinant Baculoviruses

High titer recombinant virus is critical for expression of a target protein. At the end of the 1^{st} run transfection incubation, the medium containing recombinant viruses was harvested from each 60-mm dish and all the virus-containing media were used to infect fresh naïve cells. Fresh medium was used to replace the virus stock after 1 hour infection and the cells were further incubated at 27° C. for 5-7 days (2^{nd} run amplification). Above steps were repeated until the titer of recombinant virus was high enough to express a detectable target protein. The virus stock was used for PCR to confirm the presence of the LC/A gene. The high-titered viruses were used to infect the insect Sf21 cells and the cell lysates were used to determine the presence of the LC/A protein.

(4) Determination of Recombinant Baculovirus by a Reporter Gene Assay: Beta-Glucuronidase Enzymatic Activity Assay

The transfer vector pBACgus-1 carries the gus gene encoding enzyme beta-Glucuronidase under control of the late basic protein promoter (P_{6,9}), which serves as a reporter to verify recombinant viruses by using the enzymatic reaction with its substrate X-Gluc. About five days post-transfection of each run, a 100 ul sample of the medium from each dish was taken and combined with 5 ul substrate X-Gluc (20 mg/ml). After incubation of a few hours or over-night (lower titer of viruses), recombinant pBACgus-containing viruses expressing beta-Glucuronidase was indicated by the blue staining (FIG. 3).

As shown in FIG. 3, both wild type (WT) and inactive mutant (mt) LC/A in pBACgus-1 transfer vector were incorporated into the recombinant baculoviruses as indicated by the respective medium that stained blue at the second run (6 days post infection) and the third run (5 days infection). However, they did not show blue color at the first run (5 days post transfection), which may be due to the low titer of recombinant baculovirus generated. Negative control (AcNPV vector alone) did not show any blue color at all three runs, as expected, suggesting that there were no recombinant baculoviruses generated since the essential regions for making a recombinant baculovirus are associated with the transfer plasmid.

(5) Determination of rBoNT/A-LC Expression by SDS-PAGE, Western Blotting by Anti-LC/A Antibody and Anti-His-Tag (Tagged on LC/A Gene) Monoclonal Antibody

a) Expression of rLC/A Indicated by SDS-PAGE and Coomassie Blue Staining

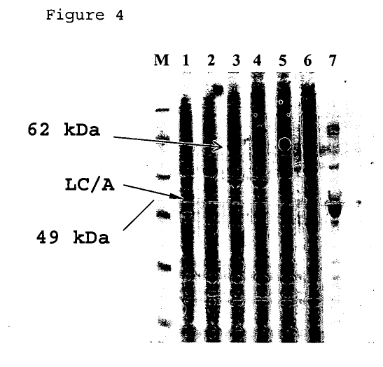

Expression of BoNT/A-LC was assessed by separation using SDS-PAGE of total cell extracts followed with the Coomassie blue staining (FIG. 4). A potential target protein migrating with the right molecular weight (50 kDa) was revealed only in presence of the cells harboring the recombinant baculoviruses of BoNT/A-LC (lane 1-4, FIG. 4), which is absent in the cells without the recombinant baculoviruses (vector alone, lane 5, FIG. 4) or in the cells alone (cells alone control, lane 6, FIG. 4). Notice that a protein migrating as 62 kDa, present only in the cells harboring pBACgus-1/LC/A but not the cells with pBAC-1/LC/A or vector alone or cells alone, is likely the reporter beta-Glucuronidase.

Methods: The 2×10⁵ cells (equal numbers of cells for all samples) were resuspended in 100 ul TE buffer (10 mM Tris-HCl, pH 8.0, 1 mM EDTA). 100 ul of 2× lysis buffer with reducing agent and proteinase inhibitors were mixed with the cell suspension. The mixture was heated at 95° C. for 5 min and immediately 20 ul of the above sample was loaded in each lane of the precast gel system (4-12% SDS-PAGE Nupage, Invitrogen). Notice that equal amount of proteins were loaded for all the lanes.

b) Expression of rLC/A was Confirmed by SDS-PAGE and Western Blotting Using Specific Anti-LC/A Polyclonal Antibody and Specific Anti-His-Tag (Tagged on the C-Terminal LC/A Gene) Monoclonal Antibody

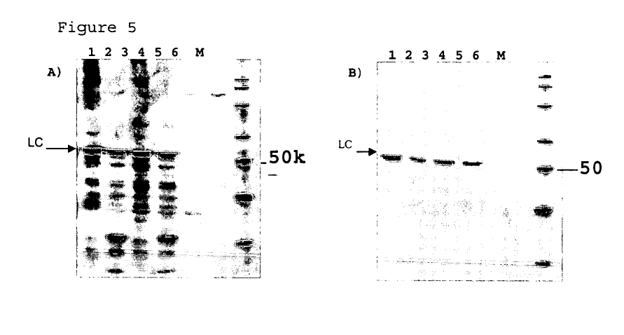

The expression of recombinant LC/A was further determined with a specific anti-LC/A polyclonal antibody (pAb) for Western blot analysis. Two duplicating protein blots were probed with either anti-LC polyclonal antibody (FIG. 5A) or anti-His tag monoclonal antibody (FIG. 5B). Both antibodies specifically recognized the 50-kDa protein only in rLC/A-containing cells (lanes 1-4, not in vector alone or cell alone controls (lanes 5 and 6, FIG. 3).

The data clearly demonstrated that we have successfully expressed both wild type and inactive mutant rBoNT/A-LC in BEVS. The experiments also indicated that the expression of recombinant BoNT/A-LC is not toxic to insect cells and BEVS is a feasible system to express an active toxin.

(6) Evaluation of the Endopeptidase Enzymatic Activity of rBoNT/A-LC, Both Wild Type and Inactive Mutant, Expressed in BEVS

The endopeptidase enzymatic activity of both wild type and mutant rBoNT/A-LC was determined by GFP-SNAP cleavage assay. In principle, this is an in vitro fluorescence release assay for quantifying the protease activity of botulinum neurotoxins. It combines the ease and simplicity of a recombinant substrate with the sensitivity that can be obtained with a fluorescent signal. It is capable of measuring the activity of BoNT/A at low picomolar concentrations.

Briefly, the high titer of recombinant viruses containing either wild type LC/A or the inactive mutant LC/A from 3^{rd} run was used to infect the insect Sf21 cells. After 3 days post-infection, cells were harvested. 1.2×10⁶ cells from each infection were pelleted and resuspended in 100 ul reaction buffer (50 mM HEPES, pH 7.4; 10 uM ZnCl₂; 0.1% (v/v) Tween-20; no DTT; protease inhibitor cocktail). Cells were lysed on ice for 45 min. After spin down the cell debris at 14,000 rpm for 10 min at 4° C., supernatant was collected and analyzed for protein concentration by the BCA assay. For each recombinant LC/A lysate, both 5 ul (3 ug) and 20 ul (12 ug) were diluted in toxin reaction buffer and added to black v-bottom 96-well plates (Whatman) in 25 ul aliquots. The procedure of GFP-SNAP assay was illustrated in previous quarterly reports (refer to Lance Steward, and Marcella Gilmore). This was the first time of application of GFP-SNAP assay on measuring LC/A activity using the whole cell lysate.

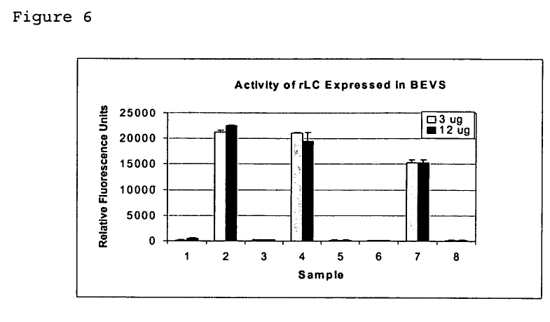

The endopeptidase enzymatic activity of baculovirally-expressed recombinant LC/A was shown in FIG. 6. The wild type LC/A, transfected in both transfer vectors pBAC-1 and pBACgus-1, showed significant high activity. There was no significant difference between the samples of 3 ug and 12 ug, suggesting that the activity of LC/A in 3 ug lysate reached the maximum. Whereas, little or no activity was shown in the inactive mutant LC/A, vector alone control, cells alone control, and substrate alone control, indicating that GFP-SNAP25 cleavage assay specifically detected the LC/A wild type. Taken together, the data of GFP-SNAP assay using the baculovirally-expressed LC/A demonstrated that active LC/A was successfully expressed in BEVS. As such, the wild type LC/A expressed in BEVS is endopeptidase enzymatically active while the inactive mutant LC was not active.

### Example 4

### Construction of BoNT/A-HC Recombinant Baculovirus Expression Vector

(1) PCR and TOPO TA Cloning

The full-length BoNT/A-HC was amplified by PCR and the amplified product was subcloned into TOPO-TA cloning vector. Total genomic DNA from C. botulinum Hall A strain was used as the template in PCR reaction. The following primers were used to generate the BoNT/A HC DNA fragment: The sense PCR primer is 5′-CA GGA TCC ATG GCA TTA AAT GAT TTA TGT ATC-3′ (SEQ ID NO: 27) with a BamHI restriction site at 5′end and the antisense PCR primer is 5′-TGT AAA CTC GAG CAG TGG CCT TTC TCC CCA TCC-3′ (SEQ ID NO: 28) with Xho I restriction site at 3′ end.

(2) Subcloning BoNT/A HC into pBAC-1 and pBACgus-1 Transfer Vectors

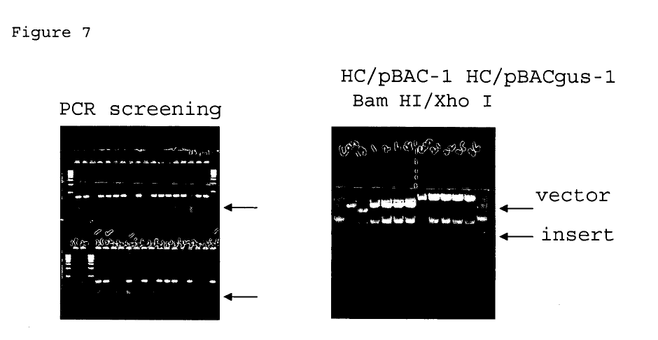

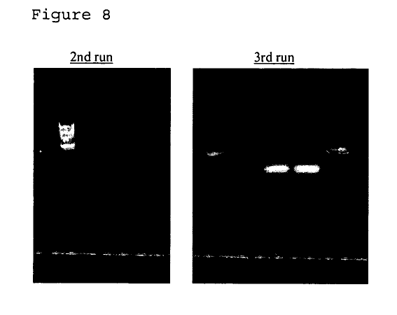

The BoNT/A HC DNA fragment (about 2.6 Kb) was cloned into pBAC-1 and pBACgus-1 transfer vectors at BamHI/XhoI sites. The right clone was identified by restriction enzyme digestion, PCR, and DNA sequencing. Subcloning of BoNT/A-HC into pBAC-1 or pBACgus-1 vector as confirmed by PCR. (FIG. 7). The insert of 2.6 kb was shown by PCR screening (the left panel, indicated by the arrow). It is also confirmed by restriction digestion (BamHI/XhoI) (the right panel): 2.6 kb is the insert and the slower migrated band is the vectors: either pBAC-1 or pBACgus-1.

(3) Co-Transfection of AcNPV and Transfer Plasmid to Making Recombinant Baculovirus In Vivo Insect Cell

The target HC gene was inserted into a transfer vector, either pBAC-1 or pBACgus-1. The transfer recombinant plasmid was co-transfected into insect host Sf9 cells with the linearized virus (AcNPV) DNA. In the transfer vector, HC gene was engineered with flanking sequences, which are homologous to the baculovirus genome. During virus replication, the target HC gene can be incorporated into the baculovirus genome at a specific locus by in vivo homologous recombination. As a result, the recombinant viruses can produce recombinant protein and also infect additional insect cells thereby producing additional recombinant viruses.

Briefly, for each transfection, 2.5×10⁶ Sf9 cells were seeded on a 60 mm dish and incubated for 20-30 min at 27° C. for cell attachment. Meanwhile, in a 1.5 ml tube, 500 ng of transfer plasmid HC gene, 100 ng of linearized AcNPV and 5 ul of Eufection transfection reagent were assembled and this DNA/Eufectin mixture was incubated at RT for 15 min. The transfection control plasmid provided with the kit was used as a positive control to verify the generation of recombinant virus. The medium instead of plasmid DNA was used as a negative control. After the DNA/Eufectin incubation was complete, 0.45 ml of medium was added to the mixture and then 0.3 ml of the mixture was transferred tol ml of medium covering the cells and incubated at 27° C. for 1 hour. Finally 6 ml of medium with serum and antibiotics was added and incubated at 27° C. for 3-4 days.

(4) Amplification of Recombinant Baculovirus

To prepare the high titer recombinant virus is critical for expression of target protein. At the end of the transfection incubation, the medium containing recombinant viruses was harvested from the 60 mm dish, and all the virus-containing medium were used to infect naive cells. Fresh medium was changed after 1 hour infection and the cells were further incubated at 27° C. for 5-7 days (2^{nd} run amplification). Above steps were repeated until the titer of recombinant virus was high enough to express detectable target protein. The high-titered viruses were used to determine the presence of the HC gene and the protein expression.

Determination of Recombinant Baculovirus

PCR Analysis

Insertion of the HC gene can be verified by PCR analysis of DNA recovered from the amplified virus stock.

As shown in FIG. 8, the recombinant virus DNA was isolated from 2^{nd} run and 3^{rd} run amplified virus. This material was used as the template; specific oligonucleotides from HC gene were designed as the PCR primers.

The 350 bp HC fragments were amplified from both #6 and #36 virus clones transfections. PCR signal from 3^{rd} run is much stronger than that from 2^{nd} run, which is probably due to the higher titer of the recombinant virus.

Liquid Overlay Assay

The transfer control plasmid and pBACgus-1 transfer plasmid provide the ability to visualize recombinants by staining with the colorimetric substrate X-Gluc, which stains for beta-glucuronidase (Gus) activity. In this assay, 40 ug of X-Gluc was added to 100 ul aliquots of the amplified virus supernatant. With the presence of Gus gene, the aliquots will turn to blue within the period of time. Positive control and #36/pBACgus-1 clones were turned to blue at 2^{nd} run and 3^{rd} run recombinant virus amplification. As similar to PCR result, signal was much stronger at the 3^{rd} run than at the 2^{nd} run because of the higher titer of the viruses.

Morphological Change of Insect Cells

Healthy insect Sf9 cells attach well to the bottom of the plate forming a clear monolayer and the cell numbers double every 72 hours. Infected cells, uniformly round, enlarged, with enlarged nuclei, do not attach well and stop dividing.

(5) Determination of rBoNT/A HC Expression

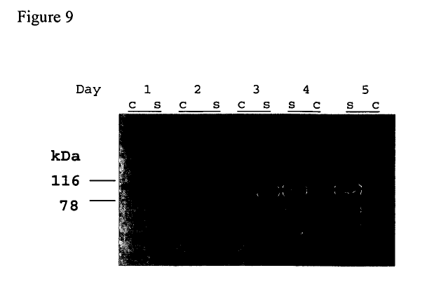

Accurate titers of virus stocks and healthy, actively dividing cells are the key to obtain the optimal protein expression. To optimize expression condition, the infection time-course was performed from day 1 to day 5. Western blotting was used to monitor the specific HC protein expression as follows. Briefly, cell lysates from day 1 to day 5 were subjected to SDS-PAGE and immumoblot analysis with anti-Toxin polyclonal antibody (1:5000 dilution) which specifically recognizes HC target protein. As shown in FIG. 9, the target protein, 100 kDa of rBoNT/A-HC was detected from day 2 post-infection. The intensity of the specific signal was increased with the increasing infection time from day 3 to day 5. No band was recognized by the anti-toxin pAb in the baculovirus vector alone (FIG. 9). In the experiment, equivalent amounts of total protein were loaded in each lane.

### Example 5

### Amplification of Recombinant Baculoviruses

High titer recombinant virus is critical for expression of a target protein. At the end of the 1^{st} run transfection incubation, the medium containing recombinant viruses was harvested from each 60-mm dish and all the virus-containing media were used to infect fresh naïve cells. Fresh medium was used to replace the virus stock after 1 hour infection and the cells were further incubated at 27° C. for 5-7 days (2^{nd} run amplification). Above steps were repeated until the titer of recombinant virus was high enough to express a detectable target protein. The virus stock was used for PCR to confirm the presence of the LC/A gene. The high-titered viruses were used to infect the insect Sf21 cells and the cell lysates were used to determine the presence of the LC/A protein.

Determination of recombinant baculovirus by a reporter gene assay: beta-Glucuronidase enzymatic activity assay. The transfer vector pBACgus-1 carries the gus gene encoding enzyme beta-Glucuronidase under control of the late basic protein promoter (P_{6,9}), which serves as a reporter to verify recombinant viruses by using the enzymatic reaction with its substrate X-Gluc. About five days post-transfection of each run, a 100 ul sample of the medium from each dish was taken and combined with 5 ul substrate X-Gluc (20 mg/ml). After incubation of a few hours or over-night (lower titer of viruses), recombinant pBACgus-containing viruses expressing beta-Glucuronidase was indicated by the blue staining.

### Example 6

### Co-Infecting Insect Cells with Recombinant LC and HC Baculoviruses, whereby the LC and the HC Forms a Disulfide Bridge

The construction and amplification of LC and HC recombinant baculovirus were shown in Examples 3 and 4. Sf21 cells were co-infected with recombinant baculovirus expressing iLC and HC. In this experiment, Sf12 cells were infected with recombinant baculovirus of iLC and HC. After three days post infection, Sf21 cells were harvested and resuspended in 300 ul of lysis buffer (10 mM Tris-Cl pH 7.5, 130 mM NaCl, 1% Triton X-100, 10 mM NaF, 10 mM NaPi, 10 mM NaPiPi, and EDTA-free protease inhibitors). After 45 minutes incubation on ice, cells were centrifuged at 14,000 rpm for 10 minutes at 4 degrees Celsius. Supernatant of each sample was collected. The protein concentration was determined by BCA protein assay. Each supernatant was mixed with equal volume of 2× lysis buffer which contained protease inhibitors with/without reducing agent. These samples were heated at 95 degrees Celsius for 5 minutes and then loaded on 4-12% SDS-Nupage gels.

In order to confirm the expression of both iLC and HC in Sf21 insect cells, Western blot assays were carried out. To achieve this, polyclonal antibodies against toxin A and LC-A were used. iLC was expressed in Sf21 cells when they were infected with 1 ml of iLC recombinant baculovirus, and also co-infected with variable volumes of iLC and HC baculovirus. Comparing to the iLC expression in sample 5, 6, 7 that were infected with 1 ml of iLC virus, the higher iLC expression level of sample 8 that was infected with 2 ml of iLC virus, and sample 9 that was infected with 3 ml of iLC virus, was observed. This suggested that higher titer of virus produces a higher expression level of target protein.

HC was expressed as well when Sf21 cells were infected with 1 ml of HC recombinant baculovirus, and also co-infected with variable volumes of iLC and HC baculovirus. The expression level of HC did not show significant difference among the cells when they were infected with 1 ml (sample 2, 8 and 9), 2 ml (sample 6) or 3 ml (sample 7) of HC recombinant baculovirus. This may result from low titer of virus.

After the confirmation of the co-expression of iLC and HC in Sf21 cells, the subsequent non-reduced Western blot assays were conducted to assess the oligomerization of iLC and HC. Anti-toxin A and anti-His tag polyclonal antibodies were used to determine iLC and HC, since they contain C-terminus His tag. The results from both anti-toxin A and anti-His tag antibodies revealed that the iLC (50 kDa) and the HC (100 kDa) dimerized to form a protein with a molecular mass of 150 kDa, the same as that of a single chain iBoNT. Furthermore, the band pattern visualized by means of anti-toxin A and anti-LC antibodies shows that the homo-oligomerization, such as iLC-iLC and HC-HC, were not detectable in the non-reduced SDS Western blots. See FIGS. 10 and 11.

### Example 7

### Expressed of BoNT/A-LC in Insect Cells with Baculovirus Expression System is Specifically Recognized by Both Anti-BoNT/A-LC pAb and His-Tag mAb

Expression of rLC/A was confirmed by SDS-PAGE and Western blotting using specific anti-LC/A polyclonal antibody and specific anti-His-tag (tagged on the C-terminal LC/A gene) monoclonal antibody.

The expression of recombinant LC/A was further determined with a specific anti-LC/A polyclonal antibody (pAb) for Western blot analysis. Two duplicating protein blots were probed with either anti-LC polyclonal antibody or anti-His tag monoclonal antibody. Both antibodies specifically recognized the 50-kDa protein only in rLC/A-containing cells.

The data clearly demonstrated that we have successfully expressed both wild serotype and inactive mutant rBoNT/A-LC in BEVS. The experiments also indicated that the expression of recombinant BoNT/A-LC is not toxic to insect cells and BEVS is a feasible system to express an active toxin.

### Example 8

### Expressed BoNT/A-LC in Insect Cells with Baculovirus Expression System Specifically Cleaves SNAP25 as Shown by GFP-SNAP25 Cleavage Assay

Evaluation of the endopeptidase enzymatic activity of rBoNT/A-LC, both wild serotype and inactive mutant, expressed in BEVS.

The endopeptidase enzymatic activity of both wild serotype and mutant rBoNT/A-LC was determined by GFP-SNAP cleavage assay. In principle, this is an in vitro fluorescence release assay for quantifying the protease activity of botulinum neurotoxins. It combines the ease and simplicity of a recombinant substrate with the sensitivity that can be obtained with a fluorescent signal. It is capable of measuring the activity of BoNT/A at low picomolar concentrations.

Briefly, the high titer of recombinant viruses containing either wild serotype LC/A or the inactive mutant LC/A from 3^{rd} run was used to infect the insect Sf21 cells. After 3 days post-infection, cells were harvested. 1.2×10⁶ cells from each infection were pelleted and resuspended in 100 ul reaction buffer (50 mM HEPES, pH 7.4; 10 uM ZnCl₂; 0.1% (v/v) Tween-20; no DTT; protease inhibitor cocktail). Cells were lysed on ice for 45 min. After spin down the cell debris at 14,000 rpm for 10 min at 4° C., supernatant was collected and analyzed for protein concentration by the BCA assay. For each recombinant LC/A lysate, both 5 ul (3 ug) and 20 ul (12 ug) were diluted in toxin reaction buffer and added to black v-bottom 96-well plates (Whatman) in 25 ul aliquots. Reagents: 2× Toxin Rxn Buffer (100 mM HEPES, pH 7.2; 0.2% (v/v) TWEEN-20; 20 μM ZnCl2; 20 mM DTT).

Assay Rinse Buffer (50 mM HEPES, pH 7.4); 8M Guanadine Hydrochloride (Pierce); Co2+ Resin (Talon Superflow Metal Affinity Resin from BD Biosciences); GFP-SNAP25 (134-206) fusion protein substrate Purified.

Procedure of LC/A as a positive control: 100 uL Rxn of 50 mM Hepes, pH 7.4, 10 mM DTT, 10 uM ZnCl₂, 0.1 mg/mL BSA, 60 ug GFP-SNAP-His, 0.0001-1.0 ug/mL rLC/A for 1 hr incubation; terminated by 8M Guanadine Hydrochloride (1 M final concentration); added 100 uL Co²⁺ resin and incubated 15 min before spin and pass over resin twice. The eluted samples were assayed to measure the fluorescent unit by absorbance of an innovative microplate reader.

The endopeptidase enzymatic activity of baculovirally-expressed recombinant LC/A was observed. The wild serotype LC/A, transfected in both transfer vectors pBAC-1 and pBACgus-1, showed significant high activity. There was no significant difference between the samples of 3 ug and 12 ug, suggesting that the activity of LC/A in 3 ug lysate reached the maximum. Whereas, little or no activity was shown in the inactive mutant LC/A, vector alone control, cells alone control, and substrate alone control, indicating that GFP-SNAP25 cleavage assay specifically detected the LC/A wild serotype. Taken together, the data of GFP-SNAP assay using the baculovirally-expressed LC/A demonstrated that active LC/A was successfully expressed in BEVS. As such, the wild serotype LC/A expressed in BEVS is endopeptidase enzymatically active while the inactive mutant LC was not active.

### Example 9

### Exemplary Methods for Co-Expressing NTNH and Active or iBoNT in Insect Cells

A second baculoviral construct expressing the NTNH gene can be used to coinfect the system of Example 3, whereby high levels of expression of recombinant LC, HC and NTNH proteins are coexpressed. In some embodiments, the cells may be infected with the construct expressing the LC, HC and the construct expressing the NTNH simultaneously. In some embodiments, the cells may be infected with the construct expressing the single chain HC, LC and the construct expressing the NTNH sequentially, in which the construct expressing the LC and HC may be infected before or after the construct expressing the NTNH.

Again using recombinant DNA technology, a transfer vector for use with baculovirus to infect Spodoptera frugiperda cells is constructed to contain the gene of interest (in this case, the gene encoding NTNH gene [residues 963-4556 of Genbank Accession U63808]). A recombinant baculovirus with the NTNH gene under the control of the promoter for the polyhedrin gene of baculovirus is obtained by recombination in the same manner as described in Example 1 or 2. The recombinant baculovirus expressing the NTNH gene thus obtained is purified and amplified, and along with the recombinant baculovirus expressing the LC and HC cDNAs, both recombinant baculoviral vectors are then used to infect cells of Spodoptera frugiperda in order to express both heterologous proteins. The co-expression of the two proteins in insect cells should produce a properly nicked iBoNT/A protein.

Once expressed, the NTNH protein may facilitate the co-expressed LC and HC to form a LC-HC disulfide bridge. Moreover, the insect cells may grow and secrete the processed di-chain BoNT of interest directly into the culture medium.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

A number of publications and patents have been cited herein. The disclosures of these publications and patents are incorporated in their entirety by reference herein. Further, the following U.S. Patents are incorporated by reference herein: Ser. No. 10/732,703 and No. 10/715,810.

What is claimed is:

1. A method of manufacturing a di-chain botulinum toxin, the method comprises expressing a botulinum toxin light chain and a botulinum toxin heavy chain separately in a same cell, whereby the light chain forms a disulfide bridge with the heavy chain to form a di-chain botulinum toxin.

2. The method of claim 1 wherein a vector is used for expressing the botulinum toxin light chain and the botulinum heavy chain in the cell.

3. The method of claim 2 wherein a single vector is used for expressing the botulinum toxin light chain and the botulinum toxin heavy chain.

4. The method of claim 2 wherein a first vector is used for expressing the botulinum toxin light chain and a second vector is used for expressing the botulinum toxin heavy chain.

5. The method of claim 2, 3 or 4 wherein the vector is a viral-based expression vector, plasmid-based expression vector, yeast expression vector, bacterial expression vector, a plant expression vector, an amphibian expression vector, a mammalian expression vector or a recombinant baculovirus vector.

6. The method of claim 2, 3, or 4 wherein the vector is a recombinant baculovirus vector.

7. The method of claim 3 wherein the vector is a recombinant baculovirus vector.

8. The method of claim 1, wherein the cell is a prokaryotic cell.

9. The method of claim 8, wherein the prokaryotic cell is an Escherichia coli cell, Clostridium botulinum cell, Clostridium tetani cell, Clostridium beratti cell, Clostridium butyricum cell, or Clostridium perfringens cell.

10. The method of claim 1, wherein the cell is a eukaryotic cell.

11. The method of claim 10, wherein the eukaryotic cell is an insect cell.

12. The method of claim 11, wherein the insect cell is a Spodoptera frugiperda cell, Aedes albopictus cell, Trichoplusia ni cell, Estigmene acrea cell, Bombyx mori cell or Drosophila melanogaster cell.

13. The method of claim 10, wherein the eukaryotic cell is a yeast cell.

14. The method of claim 13, wherein the yeast cell is a Saccharomyces cerevisiae cell, Schizosaccharomyces pombe cell, Pichia pastoris cell, Hansenula polymorpha cell, Kluyveromyces lactis cell or Yarrowia lipolytica cell.

15. The method of claim 10, wherein the eukaryotic cell is a plant cell, an amphibian cell or a mammalian cell.

16. The method of claim 1, wherein the botulinum toxin light chain is a light chain of Clostridium botulinum toxin serotypes A, B, C1, D, E, F or G.

17. The method of claim 1, wherein the botulinum toxin heavy chain is a heavy chain of Clostridium botulinum toxin serotypes A, B, C1, D, E, F or G.

18. The method of claim 1 wherein the light chain is of a serotype that is the same as that of the heavy chain serotype.

19. The method of claim 1 wherein the light chain is of a serotype that is different from the heavy chain serotype.

20. The method of claim 1 further comprises expressing one or more accessory protein in the cell, whereby the accessory protein facilitates the disulfide bridge formation between the light chain and the heavy chain.

21. The method of claim 20, wherein the accessory protein is an NTNH, HA70, HA34, HA17, GroES, GroEL, a disulfide isomerase or a heat shock protein.

22. A vector comprising a baculovirus promoter operably linked to a light chain of a botulinum toxin or a heavy chain of a botulinum toxin.

23. The vector of claim 22 wherein the promoter is a polyhedrin or polypeptide 10 (p10) promoter.

24. The vector of claim 22 wherein the light chain is a light chain of botulinum toxin serotype A, B, C1, D, E, F or G.

25. The vector of claim 22 wherein the heavy chain is a heavy chain of botulinum toxin serotype A, B, C1, D, E, F or G.

26. The vector of claim 22 which is a baculovirus vector.

27. A host cell comprising a vector of claim 23, 24, 25 or 26.

28. The host cell of claim 27 being a prokaryotic cell.

29. The host cell of claim 28, wherein the prokaryotic cell is an Escherichia coli cell, Clostridium botulinum cell, Clostridium tetani cell, Clostridium beraffi cell, Clostridium butyricum cell, or Clostridium perfringens cell.

30. The host cell of claim 27 being a eukaryotic cell.

31. The host cell of claim 30, wherein the eukaryotic cell is an insect cell.

32. The host cell of claim 31, wherein the insect cell is a Spodoptera frugiperda cell, Aedes albopictus cell, Trichoplusia ni cell, Estigmene acrea cell, Bombyx mori cell or Drosophila melanogaster cell.

33. The host cell of claim 31, wherein the insect cell is an Sf9 cell, an Sf21 cell, or a BTI-Tn-5B1-4 cell.

34. The host cell of claim 31, wherein the eukaryotic cell is a yeast cell.

35. The host cell of claim 32, wherein the yeast cell is a Saccharomyces cerevisiae cell, Schizosaccharomyces pombe cell, Pichia pastoris cell, Hansenula polymorpha cell, Kluyveromyces lactis cell or Yarrowia lipolytica cell.

36. A host cell comprising a vector operably harboring a nucleic acid sequence encoding a botulinum toxin light chain, and a nucleic acid sequence encoding a botulinum toxin heavy chain, wherein the light chain and the heavy chain are expressed in the cell as independent peptides.

37. The host cell of claim 36, wherein the cell is an insect cell.

38. The host cell of claim 36, wherein the cell is an Sf9 cell, an Sf21 cell, or a BTI-Tn-5B1-4 cell.

39. The host cell of claim 36, wherein the vector comprises a baculovirus promoter operably linked to a light chain of a botulinum toxin or a heavy chain of a botulinum toxin.

40. The host cell of claim 39, wherein the promoter is a polyhedrin or polypeptide 10 (p10) promoter.

41. The host cell of claim 39, wherein the light chain is a light chain of botulinum toxin serotype A, B, C1, D, E, F or G.

42. The host cell of claim 39, wherein the heavy chain is a heavy chain of botulinum toxin serotype A, B, C1, D, E, F or G.

43. The host cell of claim 39, wherein the vector is a baculovirus vector.

44. A cell comprising a first vector operably harboring a nucleic acid sequence encoding a botulinum toxin light chain and a second vector operably harboring a nucleic acid sequence encoding a botulinum toxin heavy chain, wherein the light chain and the heavy chain are expressed in the cell as independent peptides.

45. A di-chain botulinum toxin made by expressing a botulinum toxin light chain and a botulinum toxin heavy chain separately in a same cell, whereby the light chain forms a disulfide bridge with the heavy chain to form a di-chain botulinum toxin.

46. The toxin of claim 45 wherein a vector is used for expressing the botulinum toxin light chain and the botulinum heavy chain in the cell.

47. The toxin of claim 46 wherein a single vector is used for expressing the botulinum toxin light chain and the botulinum toxin heavy chain.

48. The toxin of claim 46 wherein a first vector is used for expressing the botulinum toxin light chain and a second vector is used for expressing the botulinum toxin heavy chain.

49. The toxin of claim 46, 47 or 48 wherein the vector is a viral-based expression vector, plasmid-based expression vector, yeast expression vector, bacterial expression vector, a plant expression vector, an amphibian expression vector, a mammalian expression vector or a recombinant baculovirus vector.

50. The toxin of claim 46, 47 or 48 wherein the vector is a recombinant baculovirus vector.

51. The toxin of claim 45, wherein the cell is a prokaryotic cell.

52. The toxin of claim 51, wherein the prokaryotic cell is an Escherichia coli cell, Clostridium botulinum cell, Clostridium tetani cell, Clostridium beratti cell, Clostridium butyricum cell, or Clostridium perfringens cell.

53. The toxin of claim 45, wherein the cell is a eukaryotic cell.

54. The toxin of claim 53, wherein the eukaryotic cell is an insect cell.

55. The toxin of claim 54, wherein the insect cell is a Spodoptera frugiperda cell, Aedes albopictus cell, Trichoplusia ni cell, Estigmene acrea cell, Bombyx mori cell or Drosophila melanogaster cell.

56. The toxin of claim 53, wherein the eukaryotic cell is a yeast cell.

57. The toxin of claim 56, wherein the yeast cell is a Saccharomyces cerevisiae cell, Schizosaccharomyces pombe cell, Pichia pastoris cell, Hansenula polymorpha cell, Kluyveromyces lactis cell or Yarrowia lipolytica cell.

58. The toxin of claim 53, wherein the eukaryotic cell is a plant cell, an amphibian cell or a mammalian cell.

59. The method of claim 45, wherein the botulinum toxin light chain is a light chain of Clostridium botulinum toxin serotypes A, B, C1, D, E, F or G.

60. The method of claim 45, wherein the botulinum toxin heavy chain is a heavy chain of Clostridium botulinum toxin serotypes A, B, C1, D, E, F or G.

61. The method of claim 45 wherein the light chain is of a serotype that is the same as that of the heavy chain serotype.

62. The method of claim 45 wherein the light chain is of a serotype that is different from the heavy chain serotype.

63. The method of claim 45 further comprises expressing one or more accessory protein in the cell, whereby the accessory protein facilitates the disulfide bridge formation between the light chain and the heavy chain.

64. The method of claim 63, wherein the accessory protein is an NTNH, HA70, HA34, HA17, GroES, GroEL, a disulfide isomerase or a heat shock protein.


SEQUENCE LISTING (48 sequences)

SEQ ID NO: 1 - protein, 5 aa, Artificial Sequence
  zinc binding motif; X = any amino acid residue
HEXXH

SEQ ID NO: 2 - protein, 5 aa, Artificial Sequence
  zinc binding motif; X = any amino acid residue
GTXXN

SEQ ID NO: 3 - protein, 4 aa, Artificial Sequence
  potential sites of N-glycosylation on the surface of Botulinum Toxin Type A
NLTR

SEQ ID NO: 4 - protein, 4 aa, Artificial Sequence
  potential sites of N-glycosylation on the surface of Botulinum Toxin Type A
NYTI

SEQ ID NO: 5 - protein, 4 aa, Artificial Sequence
  potential sites of N-glycosylation on the surface of Botulinum Toxin Type A
NFTK

SEQ ID NO: 6 - protein, 4 aa, Artificial Sequence
  potential sites of N-glycosylation on the surface of Botulinum Toxin Type A
NFTG

SEQ ID NO: 7 - protein, 4 aa, Artificial Sequence
  potential sites of N-glycosylation on the surface of Botulinum Toxin Type A
NNSG

SEQ ID NO: 8 - protein, 4 aa, Artificial Sequence
  potential sites of N-glycosylation on the surface of Botulinum Toxin Type A
NISD

SEQ ID NO: 9 - protein, 4 aa, Artificial Sequence
  potential sites of N-glycosylation on the surface of Botulinum Toxin Type A
NASQ

SEQ ID NO: 10 - protein, 4 aa, Artificial Sequence
  potential sites of N-glycosylation on the surface of Botulinum Toxin Type A
NLSQ

SEQ ID NO: 11 - protein, 4 aa, Artificial Sequence
  potential sites of N-glycosylation on the surface of Botulinum Toxin Type A
NLTR

SEQ ID NO: 12 - protein, 4 aa, Artificial Sequence
  potential sites of N-glycosylation on the surface of Botulinum Toxin Type A
NYTI

SEQ ID NO: 13 - protein, 4 aa, Artificial Sequence
  potential sites of N-glycosylation on the surface of Botulinum Toxin Type A
NFTK

SEQ ID NO: 14 - protein, 4 aa, Artificial Sequence
  potential sites of N-glycosylation on the surface of Botulinum Toxin Type A
NFTG

SEQ ID NO: 15 - protein, 4 aa, Artificial Sequence
  potential sites of N-glycosylation on the surface of Botulinum Toxin Type A
NNSG

SEQ ID NO: 16 - protein, 4 aa, Artificial Sequence
  potential sites of N-glycosylation on the surface of Botulinum Toxin Type A
NISD

SEQ ID NO: 17 - protein, 4 aa, Artificial Sequence
  potential sites of N-glycosylation on the surface of Botulinum Toxin Type A
NASQ

SEQ ID NO: 18 - protein, 4 aa, Artificial Sequence
  potential sites of N-glycosylation on the surface of Botulinum Toxin Type A
NLSQ

SEQ ID NO: 19 - protein, 4 aa, Artificial Sequence
  potential sites of N-glycosylation on the surface of Botulinum Toxin Type E
NLSG

SEQ ID NO: 20 - protein, 4 aa, Artificial Sequence
  potential sites of N-glycosylation on the surface of Botulinum Toxin Type E
NGSG

SEQ ID NO: 21 - protein, 4 aa, Artificial Sequence
  potential sites of N-glycosylation on the surface of Botulinum Toxin Type E
NSSN

SEQ ID NO: 22 - protein, 4 aa, Artificial Sequence
  potential sites of N-glycosylation on the surface of Botulinum Toxin Type E
NISL

SEQ ID NO: 23 - protein, 4 aa, Artificial Sequence
  potential sites of N-glycosylation on the surface of Botulinum Toxin Type E
NDSI

SEQ ID NO: 24 - protein, 4 aa, Artificial Sequence
  potential sites of N-glycosylation on the surface of Botulinum Toxin Type E
NISE

SEQ ID NO: 25 - DNA, 32 bp, Artificial Sequence
  sense PCR primer to Botulinum Toxin Type A
caggatccatgccatttgttaataaacaattt

SEQ ID NO: 26 - DNA, 34 bp, Artificial Sequence
  antisense PCR primer to Botulinum Toxin Type A
ccccctcgagcttattgtatcctttatctaatga

SEQ ID NO: 27 - DNA, 32 bp, Artificial Sequence
  sense PCR primer to Botulinum Toxin Type A
caggatccatggcattaaatgatttatgtatc

SEQ ID NO: 28 - DNA, 33 bp, Artificial Sequence
  antisense PCR primer to Botulinum Toxin Type A
tgtaaactcgagcagtggcctttctccccatcc

SEQ ID NO: 29 - DNA, 1312 bp, Artificial Sequence
  nucleic sequence of LC
atgccatttgttaataaacaatttaattataaagatcctgtaaatggtgttgatattgcttatataaaaattccaaatgcaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtaagagggataataacttcta

SEQ ID NO: 30 - DNA, 2547 bp, Artificial Sequence
  nucleic acid sequence of HC
gcattaaatgatttatgtatcaaagttaataattgggacttgttttttagtccttcagaagataattttactaatgatctaaataaaggagaagaaattacatctgatactaatatagaagcagcagaagaaaatattagtttagatttaatacaacaatattatttaacctttaattttgataatgaacctgaaaatatttcaatagaaaatctttcaagtgacattataggccaattagaacttatgcctaatatagaaagatttcctaatggaaaaaagtatgagttagataaatatactatgttccattatcttcgtgctcaagaatttgaacatggtaaatctaggattgctttaacaaattctgttaacgaagcattattaaatcctagtcgtgtttatacatttttttcttcagactatgtaaagaaagttaataaagctacggaggcagctatgtttttaggctgggtagaacaattagtatatgattttaccgatgaaactagcgaagtaagtactacggataaaattgcggatataactataattattccatatataggacctgctttaaatataggtaatatgttatataaagatgattttgtaggtgctttaatattttcaggagctgttattctgttagaatttataccagagattgcaatacctgtattaggtacttttgcacttgtatcatatattgcgaataaggttctaaccgttcaaacaatagataatgctttaagtaaaagaaatgaaaaatgggatgaggtctataaatatatagtaacaaattggttagcaaaggttaatacacagattgatctaataagaaaaaaaatgaaagaagctttagaaaatcaagcagaagcaacaaaggctataataaactatcagtataatcaatatactgaggaagagaaaaataatattaattttaatattgatgatttaagttcgaaacttaatgagtctataaataaagctatgattaatataaataaatttttgaatcaatgctctgtttcatatttaatgaattctatgatcccttatggtgttaaacggttagaagattttgatgctagtcttaaagatgcattattaaagtatatatatgataatagaggaactttaattggtcaagtagatagattaaaagataaagttaataatacacttagtacagatataccttttcagctttccaaatacgtagataatcaaagattattatctacatttactgaatatattaagaatattattaatacttctatattgaatttaagatatgaaagtaatcatttaatagacttatctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtattttaaaagacttttggggtgattatttacaatatgataaaccatactatatgttaaatttatatgatccaaataaatatgtcgatgtaaataatgtaggtattagaggttatatgtatcttaaagggcctagaggtagcgtaatgactacaaacatttatttaaattcaagtttgtatagggggacaaaatttattataaaaaaatatgcttctggaaataaagataatattgttagaaataatgatcgtgtatatattaatgtagtagttaaaaataaagaatataggttagctactaatgcgtcacaggcaggcgtagaaaaaatactaagtgcattagaaatacctgatgtaggaaatctaagtcaagtagtagtaatgaagtcaaaaaatgatcaaggaataacaaataaatgcaaaatgaatttacaagataataatgggaatgatataggctttataggatttcatcagtttaataatatagctaaactagtagcaagtaattggtataatagacaaatagaaagatctagtaggactttgggttgctcatgggaatttattcctgtagatgatggatggggagaaaggccactgtaa

SEQ ID NO: 31 - DNA, 1320 bp, Artificial Sequence
  nucleic acid sequence of LC
ccagtaacaataaataattttaattataatgatccaatagataatgataatataataatgatggaaccaccatttgcaagaggaacaggaagatattataaagcatttaaaataacagatagaatatggataataccagaaagatatacatttggatataaaccagaagattttaataaaagtagtggaatatttaatagagatgtatgtgaatattatgatccagattatcttaatacaaatgataaaaaaaatatattttttcaaacacttataaaactttttaatagaataaaaagtaaaccacttggagaaaaacttcttgaaatgataataaatggaataccatatcttggagatagaagagtaccacttgaagaatttaatacaaatatagcaagtgtaacagtaaataaacttataagtaatccaggagaagtagaaagaaaaaaaggaatatttgcaaatcttataatatttggaccaggaccagtacttaatgaaaatgaaacaatagatataggaatacaaaatcattttgcaagtagagaaggatttggaggaataatgcaaatgaaattttgtccagaatatgtaagtgtatttaataatgtacaagaaaataaaggagcaagtatatttaatagaagaggatattttagtgatccagcacttatacttatgcatgaacttatacatgtacttcatggactttatggaataaaagtagatgatcttccaatagtaccaaatgaaaaaaaattttttatgcaaagtacagatacaatacaagcagaagaactttatacatttggaggacaagatccaagtataataagtccaagtacagataaaagtatatatgataaagtacttcaaaattttagaggaatagtagatagacttaataaagtacttgtatgtataagtgatccaaatataaatataaatatatataaaaataaatttaaagataaatataaatttgtagaagatagtgaaggaaaatatagtatagatgtagaaagttttaataaactttataaaagtcttatgcttggatttacagaaataaatatagcagaaaattataaaataaaaacaagagcaagttattttagtgatagtcttccaccagtaaaaataaaaaatcttcttgataatgaaatatatacaatagaagaaggatttaatataagtgataaaaatatgggaaaagaatatagaggacaaaataaagcaataaataaacaagcatatgaagaaataagtaaagaacatcttgcagtatataaaatacaaatgtgtaaaagtgtaaaa

SEQ ID NO: 32 - DNA, 2550 bp, Artificial Sequence
  nucleic acid sequence of HC
gtaccaggaatatgtatagatgtagataatgaaaatcttttttttatagcagataaaaatagttttagtgatgatcttagtaaaaatgaaagagtagaatataatacacaaaataattatataggaaatgattttccaataaatgaacttatacttgatacagatcttataagtaaaatagaacttccaagtgaaaatacagaaagtcttacagattttaatgtagatgtaccagtatatgaaaaacaaccagcaataaaaaaagtatttacagatgaaaatacaatatttcaatatctttatagtcaaacatttccacttaatataagagatataagtcttacaagtagttttgatgatgcacttcttgtaagtagtaaagtatatagtttttttagtatggattatataaaaacagcaaataaagtagtagaagcaggactttttgcaggatgggtaaaacaaatagtagatgattttgtaatagaagcaaataaaagtagtacaatggataaaatagcagatataagtcttatagtaccatatataggacttgcacttaatgtaggagatgaaacagcaaaaggaaattttgaaagtgcatttgaaatagcaggaagtagtatacttcttgaatttataccagaacttcttataccagtagtaggagtatttcttcttgaaagttatatagataataaaaataaaataataaaaacaatagataatgcacttacaaaaagagtagaaaaatggatagatatgtatggacttatagtagcacaatggcttagtacagtaaatacacaattttatacaataaaagaaggaatgtataaagcacttaattatcaagcacaagcacttgaagaaataataaaatataaatataatatatatagtgaagaagaaaaaagtaatataaatataaattttaatgatataaatagtaaacttaatgatggaataaatcaagcaatggataatataaatgattttataaatgaatgtagtgtaagttatcttatgaaaaaaatgataccacttgcagtaaaaaaacttcttgattttgataatacacttaaaaaaaatcttcttaattatatagatgaaaataaactttatcttataggaagtgtagaagatgaaaaaagtaaagtagataaatatcttaaaacaataataccatttgatcttagtacatatagtaatatagaaatacttataaaaatatttaataaatataatagtgaaatacttaataatataatacttaatcttagatatagagataataatcttatagatcttagtggatatggagcaaaagtagaagtatatgatggagtaaaacttaatgataaaaatcaatttaaacttacaagtagtgcagatagtaaaataagagtaacacaaaatcaaaatataatatttaatagtatgtttcttgattttagtgtaagtttttggataagaataccaaaatatagaaatgatgatatacaaaattatatacataatgaatatacaataataaattgtatgaaaaataatagtggatggaaaataagtataagaggaaatagaataatatggacacttatagatataaatggaaaaacaaaaagtgtattttttgaatataatataagagaagatataagtgaatatataaatagatggttttttgtaacaataacaaataatcttgataatgcaaaaatatatataaatggaacacttgaaagtaatatggatataaaagatataggagaagtaatagtaaatggagaaataacatttaaacttgatggagatgtagatagaacacaatttatatggatgaaatattttagtatatttaatacacaacttaatcaaagtaatataaaagaaatatataaaatacaaagttatagtgaatatcttaaagatttttggggaaatccacttatgtataataaagaatattatatgtttaatgcaggaaataaaaatagttatataaaacttgtaaaagatagtagtgtaggagaaatacttataagaagtaaatataatcaaaatagtaattatataaattatagaaatctttatataggagaaaaatttataataagaagagaaagtaatagtcaaagtataaatgatgatatagtaagaaaagaagattatatacatcttgatcttgtacttcatcatgaagaatggagagtatatgcatataaatattttaaagaacaagaagaaaaactttttcttagtataataagtgatagtaatgaattttataaaacaatagaaataaaagaatatgatgaacaaccaagttatagttgtcaacttctttttaaaaaagatgaagaaagtacagatgatataggacttataggaatacatagattttatgaaagtggagtacttagaaaaaaatataaagattatttttgtataagtaaatggtatcttaaagaagtaaaaagaaaaccatataaaagtaatcttggatgtaattggcaatttataccaaaagatgaaggatggacagaa

SEQ ID NO: 33 - DNA, 1344 bp, Artificial Sequence
  nucleic acid sequence of LC
ccaataacaataaataattttaattatagtgatccagtagataataaaaatatactttatcttgatacacatcttaatacacttgcaaatgaaccagaaaaagcatttagaataacaggaaatatatgggtaataccagatagatttagtagaaatagtaatccaaatcttaataaaccaccaagagtaacaagtccaaaaagtggatattatgatccaaattatcttagtacagatagtgataaagatccatttcttaaagaaataataaaactttttaaaagaataaatagtagagaaataggagaagaacttatatatagacttagtacagatataccatttccaggaaataataatacaccaataaatacatttgattttgatgtagattttaatagtgtagatgtaaaaacaagacaaggaaataattgggtaaaaacaggaagtataaatccaagtgtaataataacaggaccaagagaaaatataatagatccagaaacaagtacatttaaacttacaaataatacatttgcagcacaagaaggatttggagcacttagtataataagtataagtccaagatttatgcttacatatagtaatgcaacaaatgatgtaggagaaggaagatttagtaaaagtgaattttgtatggatccaatacttatacttatgcatgaacttaatcatgcaatgcataatctttatggaatagcaataccaaatgatcaaacaataagtagtgtaacaagtaatatattttatagtcaatataatgtaaaacttgaatatgcagaaatatatgcatttggaggaccaacaatagatcttataccaaaaagtgcaagaaaatattttgaagaaaaagcacttgattattatagaagtatagcaaaaagacttaatagtataacaacagcaaatccaagtagttttaataaatatataggagaatataaacaaaaacttataagaaaatatagatttgtagtagaaagtagtggagaagtaacagtaaatagaaataaatttgtagaactttataatgaacttacacaaatatttacagaatttaattatgcaaaaatatataatgtacaaaatagaaaaatatatcttagtaatgtatatacaccagtaacagcaaatatacttgatgataatgtatatgatatacaaaatggatttaatataccaaaaagtaatcttaatgtactttttatgggacaaaatcttagtagaaatccagcacttagaaaagtaaatccagaaaatatgctttatctttttacaaaattttgtcataaagcaatagatggaagaagtctttataataaa

SEQ ID NO: 34 - DNA, 2526 bp, Artificial Sequence
  nucleic acid sequence of HC
acacttgattgtagagaacttcttgtaaaaaatacagatcttccatttataggagatataagtgatgtaaaaacagatatatttcttagaaaagatataaatgaagaaacagaagtaatatattatccagataatgtaagtgtagatcaagtaatacttagtaaaaatacaagtgaacatggacaacttgatcttctttatccaagtatagatagtgaaagtgaaatacttccaggagaaaatcaagtattttatgataatagaacacaaaatgtagattatcttaatagttattattatcttgaaagtcaaaaacttagtgataatgtagaagattttacatttacaagaagtatagaagaagcacttgataatagtgcaaaagtatatacatattttccaacacttgcaaataaagtaaatgcaggagtacaaggaggactttttcttatgtgggcaaatgatgtagtagaagattttacaacaaatatacttagaaaagatacacttgataaaataagtgatgtaagtgcaataataccatatataggaccagcacttaatataagtaatagtgtaagaagaggaaattttacagaagcatttgcagtaacaggagtaacaatacttcttgaagcatttccagaatttacaataccagcacttggagcatttgtaatatatagtaaagtacaagaaagaaatgaaataataaaaacaatagataattgtcttgaacaaagaataaaaagatggaaagatagttatgaatggatgatgggaacatggcttagtagaataataacacaatttaataatataagttatcaaatgtatgatagtcttaattatcaagcaggagcaataaaagcaaaaatagatcttgaatataaaaaatatagtggaagtgataaagaaaatataaaaagtcaagtagaaaatcttaaaaatagtcttgatgtaaaaataagtgaagcaatgaataatataaataaatttataagagaatgtagtgtaacatatctttttaaaaatatgcttccaaaagtaatagatgaacttaatgaatttgatagaaatacaaaagcaaaacttataaatcttatagatagtcataatataatacttgtaggagaagtagataaacttaaagcaaaagtaaataatagttttcaaaatacaataccatttaatatatttagttatacaaataatagtcttcttaaagatataataaatgaatattttaataatataaatgatagtaaaatacttagtcttcaaaatagaaaaaatacacttgtagatacaagtggatataatgcagaagtaagtgaagaaggagatgtacaacttaatccaatatttccatttgattttaaacttggaagtagtggagaagatagaggaaaagtaatagtaacacaaaatgaaaatatagtatataatagtatgtatgaaagttttagtataagtttttggataagaataaataaatgggtaagtaatcttccaggatatacaataatagatagtgtaaaaaataatagtggatggagtataggaataataagtaattttcttgtatttacacttaaacaaaatgaagatagtgaacaaagtataaattttagttatgatataagtaataatgcaccaggatataataaatggttttttgtaacagtaacaaataatatgatgggaaatatgaaaatatatataaatggaaaacttatagatacaataaaagtaaaagaacttacaggaataaattttagtaaaacaataacatttgaaataaataaaataccagatacaggacttataacaagtgatagtgataatataaatatgtggataagagatttttatatatttgcaaaagaacttgatggaaaagatataaatatactttttaatagtcttcaatatacaaatgtagtaaaagattattggggaaatgatcttagatataataaagaatattatatggtaaatatagattatcttaatagatatatgtatgcaaatagtagacaaatagtatttaatacaagaagaaataataatgattttaatgaaggatataaaataataataaaaagaataagaggaaatacaaatgatacaagagtaagaggaggagatatactttattttgatatgacaataaataataaagcatataatctttttatgaaaaatgaaacaatgtatgcagataatcatagtacagaagatatatatgcaataggacttagagaacaaacaaaagatataaatgataatataatatttcaaatacaaccaatgaataatacatattattatgcaagtcaaatatttaaaagtaattttaatggagaaaatataagtggaatatgtagtataggaacatatagatttagacttggaggagattggtatagacataattatcttgtaccaacagtaaaacaaggaaattatgcaagtcttcttgaaagtacaagtacacattggggatttgtaccagtaagtgaa

SEQ ID NO: 35 - DNA, 1326 bp, Artificial Sequence
  nucleic acid sequence of LC
atgacatggccagtaaaagattttaattatagtgatccagtaaatgataatgatatactttatcttagaataccacaaaataaacttataacaacaccagtaaaagcatttatgataacacaaaatatatgggtaataccagaaagatttagtagtgatacaaatccaagtcttagtaaaccaccaagaccaacaagtaaatatcaaagttattatgatccaagttatcttagtacagatgaacaaaaagatacatttcttaaaggaataataaaactttttaaaagaataaatgaaagagatataggaaaaaaacttataaattatcttgtagtaggaagtccatttatgggagatagtagtacaccagaagatacatttgattttacaagacatacaacaaatatagcagtagaaaaatttgaaaatggaagttggaaagtaacaaatataataacaccaagtgtacttatatttggaccacttccaaatatacttgattatacagcaagtcttacacttcaaggacaacaaagtaatccaagttttgaaggatttggaacacttagtatacttaaagtagcaccagaatttcttcttacatttagtgatgtaacaagtaatcaaagtagtgcagtacttggaaaaagtatattttgtatggatccagtaatagcacttatgcatgaacttacacatagtcttcatcaactttatggaataaatataccaagtgataaaagaataagaccacaagtaagtgaaggattttttagtcaagatggaccaaatgtacaatttgaagaactttatacatttggaggacttgatgtagaaataataccacaaatagaaagaagtcaacttagagaaaaagcacttggacattataaagatatagcaaaaagacttaataatataaataaaacaataccaagtagttggataagtaatatagataaatataaaaaaatatttagtgaaaaatataattttgataaagataatacaggaaattttgtagtaaatatagataaatttaatagtctttatagtgatcttacaaatgtaatgagtgaagtagtatatagtagtcaatataatgtaaaaaatagaacacattattttagtagacattatcttccagtatttgcaaatatacttgatgataatatatatacaataagagatggatttaatcttacaaataaaggatttaatatagaaaatagtggacaaaatatagaaagaaatccagcacttcaaaaacttagtagtgaaagtgtagtagatctttttacaaaagtatgtcttagacttacaaaa

SEQ ID NO: 36 - DNA, 2502 bp, Artificial Sequence
  nucleic acid sequence of HC
aatagtagagatgatagtacatgtataaaagtaaaaaataatagacttccatatgtagcagataaagatagtataagtcaagaaatatttgaaaataaaataataacagatgaaacaaatgtacaaaattatagtgataaatttagtcttgatgaaagtatacttgatggacaagtaccaataaatccagaaatagtagatccacttcttccaaatgtaaatatggaaccacttaatcttccaggagaagaaatagtattttatgatgatataacaaaatatgtagattatcttaatagttattattatcttgaaagtcaaaaacttagtaataatgtagaaaatataacacttacaacaagtgtagaagaagcacttggatatagtaataaaatatatacatttcttccaagtcttgcagaaaaagtaaataaaggagtacaagcaggactttttcttaattgggcaaatgaagtagtagaagattttacaacaaatataatgaaaaaagatacacttgataaaataagtgatgtaagtgtaataataccatatataggaccagcacttaatataggaaatagtgcacttagaggaaattttaatcaagcatttgcaacagcaggagtagcatttcttcttgaaggatttccagaatttacaataccagcacttggagtatttacattttatagtagtatacaagaaagagaaaaaataataaaaacaatagaaaattgtcttgaacaaagagtaaaaagatggaaagatagttatcaatggatggtaagtaattggcttagtagaataacaacacaatttaatcatataaattatcaaatgtatgatagtcttagttatcaagcagatgcaataaaagcaaaaatagatcttgaatataaaaaatatagtggaagtgataaagaaaatataaaaagtcaagtagaaaatcttaaaaatagtcttgatgtaaaaataagtgaagcaatgaataatataaataaatttataagagaatgtagtgtaacatatctttttaaaaatatgcttccaaaagtaatagatgaacttaataaatttgatcttagaacaaaaacagaacttataaatcttatagatagtcataatataatacttgtaggagaagtagatagacttaaagcaaaagtaaatgaaagttttgaaaatacaatgccatttaatatatttagttatacaaataatagtcttcttaaagatataataaatgaatattttaatagtataaatgatagtaaaatacttagtcttcaaaataaaaaaaatgcacttgtagatacaagtggatataatgcagaagtaagagtaggagataatgtacaacttaatacaatatatacaaatgattttaaacttagtagtagtggagataaaataatagtaaatcttaataataatatactttatagtgcaatatatgaaaatagtagtgtaagtttttggataaaaataagtaaagatcttacaaatagtcataatgaatatacaataataaatagtatagaacaaaatagtggatggaaactttgtataagaaatggaaatatagaatggatacttcaagatgtaaatagaaaatataaaagtcttatatttgattatagtgaaagtcttagtcatacaggatatacaaataaatggttttttgtaacaataacaaataatataatgggatatatgaaactttatataaatggagaacttaaacaaagtcaaaaaatagaagatcttgatgaagtaaaacttgataaaacaatagtatttggaatagatgaaaatatagatgaaaatcaaatgctttggataagagattttaatatatttagtaaagaacttagtaatgaagatataaatatagtatatgaaggacaaatacttagaaatgtaataaaagattattggggaaatccacttaaatttgatacagaatattatataataaatgataattatatagatagatatatagcaccagaaagtaatgtacttgtacttgtacaatatccagatagaagtaaactttatacaggaaatccaataacaataaaaagtgtaagtgataaaaatccatatagtagaatacttaatggagataatataatacttcatatgctttataatagtagaaaatatatgataataagagatacagatacaatatatgcaacacaaggaggagaatgtagtcaaaattgtgtatatgcacttaaacttcaaagtaatcttggaaattatggaataggaatatttagtataaaaaatatagtaagtaaaaataaatattgtagtcaaatatttagtagttttagagaaaatacaatgcttcttgcagatatatataaaccatggagatttagttttaaaaatgcatatacaccagtagcagtaacaaattatgaaacaaaacttcttagtacaagtagtttttggaaatttataagtagagatccaggatgggtagaa

SEQ ID NO: 37 - DNA, 1269 bp, Artificial Sequence
  nucleic acid sequence of LC
gatccccaaaaattaatagttttaattataatgatcctgttaatgatagaacaattttatatattaaaccaggcggttgtcaagaattttataaatcatttaatattatgaaaaatatttggataattccagagagaaatgtaattggtacaaccccccaagattttcatccgcctacttcattaaaaaatggagatagtagttattatgaccctaattatttacaaagtgatgaagaaaaggatagatttttaaaaatagtcacaaaaatatttaatagaataaataataatctttcaggagggattttattagaagaactgtcaaaagctaatccatatttagggaatgataatactccagataatcaattccatattggtgatgcatcagcagttgagattaaattctcaaatggtagccaagacatactattacctaatgttattataatgggagcagagcctgatttatttgaaactaacagttccaatatttctctaagaaataattatatgccaagcaatcacggttttggatcaatagctatagtaacattctcacctgaatattcttttagatttaatgataatagtatgaatgaatttattcaagatcctgctcttacattaatgcatgaattaatacattcattacatggactatatggggctaaagggattactacaaagtatactataacacaaaaacaaaatcccctaataacaaatataagaggtacaaatattgaagaattcttaacttttggaggtactgatttaaacattattactagtgctcagtccaatgatatctatactaatcttctagctgattataaaaaaatagcgtctaaacttagcaaagtacaagtatctaatccactacttaatccttataaagatgtttttgaagcaaagtatggattagataaagatgctagcggaatttattcggtaaatataaacaaatttaatgatatttttaaaaaattatacagctttacggaatttgatttagcaactaaatttcaagttaaatgtaggcaaacttatattggacagtataaatacttcaaactttcaaacttgttaaatgattctatttataatatatcagaaggctataatataaataatttaaaggtaaattttagaggacagaatgcaaatttaaatcctagaattattacaccaattacaggtagaggactagtaaaaaaaatcattagattttgtaaaaatattgtttctgtaaaaggcataagga

SEQ ID NO: 38 - DNA, 2490 bp, Artificial Sequence
  nucleic acid sequence of HC
aaaagtatctgtatcgaaatcaataatggcgaactgtttttcgtcgcatctgaaaactcgtataacgatgacaatatcaacacaccgaaagaaattgatgacactgtcacttctaacaacaattacgaaaacgacctggaccaggtgatcctcaatttcaatagcgaaagcgcacccggcctgagcgatgaaaaacttaatctcacgattcagaacgacgcctacattccaaaatacgatagtaatggtacatctgatattgaacagcatgatgtcaacgaattaaatgttttcttttacctcgatgcccagaaagtgccggaaggtgagaacaacgtaaatctgacctcttcgattgatacggcattattagaacagccgaaaatttatactttcttttcgtccgaatttattaacaatgttaacaaaccggttcaagcggcgttattcgtttcctggattcagcaagttcttgtagattttacaaccgaggctaatcagaagagcacggtggataagatcgccgacatcagcatcgtcgtgccctacattggtttggcattaaacattggtaatgaggcgcaaaaggggaactttaaagacgccctggaattattaggagcaggtattctgctggagttcgaacctgagctgctgattccgactattttagtgttcaccattaaatccttcttaggctctagtgacaacaaaaataaagtgattaaagcgatcaataatgcccttaaagaacgtgatgagaaatggaaagaagtctactccttcattgtctcaaattggatgacgaaaatcaacacgcagtttaataaacgcaaagaacagatgtatcaggcgctgcaaaaccaggttaatgcgatcaagacaattattgaatctaagtacaactcgtacaccctggaggagaaaaatgaactgactaataagtacgatattaaacaaatcgaaaacgaattgaatcagaaagtctccatcgctatgaacaatatcgatcgctttctgaccgaaagctctatttcctatttgatgaaacttatcaatgaagtcaaaatcaacaaacttcgcgaatatgatgagaacgtaaaaacgtacctgctcaattatattattcaacatgggtcgattctgggcgagtctcaacaagaattgaactcgatggtgacggatactttgaataactcgattccgtttaaattatcgtcatacaccgatgataaaattcttatctcgtacttcaacaaattctttaagcggatcaaaagcagcagcgtccttaatatgcgctataaaaacgataagtacgtagatacgtctggatacgacagtaacattaatattaatggggacgtctataaatatccgacaaataaaaaccaattcgggatttataatgataaactttcggaggtgaacatcagccagaacgattatattatttacgataataaatacaaaaacttcagcatttctttttgggtgcgtatcccaaattacgacaacaaaattgtgaacgtgaataacgaatacacgatcattaattgcatgcgcgataacaattctggttggaaagttagcctgaatcacaatgagattatctggactcttcaggacaatgctggtatcaaccaaaaattagcgttcaactacggtaatgccaacggtatttctgactacatcaataagtggatctttgtgaccatcaccaatgaccgcctcggcgatagcaagctgtacattaacggtaacctgatcgaccagaaatctattctgaacctgggtaacattcacgtaagtgacaacatcctttttaaaattgtcaattgctcgtatactcgttatatcggcattcgctatttcaatattttcgacaaagaactggatgagacggaaatccagactctgtattctaacgaaccgaacaccaacatcctgaaggacttttgggggaattatcttctctacgataaagagtactaccttcttaacgtgttgaagccgaacaacttcattgatcgtcgtaaggatagcaccttgagcattaacaacattcgtagcaccattttactggcaaaccgcctgtacagcggcattaaagtcaaaattcagcgtgtcaataactccagtacgaatgacaatctggtgcggaaaaatgaccaagtctatattaactttgtcgcaagcaaaactcacctctttccattatatgcggatacagctaccaccaataaagaaaaaactattaaaatctcctcttccgggaaccgctttaatcaggtggtagttatgaactcggtcggcaacaattgtactatgaattttaaaaataataacggcaataacatcggcctgctgggcttcaaagctgatacagttgtggccagcacctggtattacacccacatgcgtgatcataccaatagtaatggctgcttttggaattttatttctgaagagcacggctggcaagaaaaa

SEQ ID NO: 39 - DNA, 1308 bp, Artificial Sequence
  nucleic acid sequence of LC
atgccagtagcaataaatagttttaattataatgatccagtaaatgatgatacaatactttatatgcaaataccatatgaagaaaaaagtaaaaaatattataaagcatttgaaataatgagaaatgtatggataataccagaaagaaatacaataggaacaaatccaagtgattttgatccaccagcaagtcttaaaaatggaagtagtgcatattatgatccaaattatcttacaacagatgcagaaaaagatagatatcttaaaacaacaataaaactttttaaaagaataaatagtaatccagcaggaaaagtacttcttcaagaaataagttatgcaaaaccatatcttggaaatgatcatacaccaatagatgaatttagtccagtaacaagaacaacaagtgtaaatataaaacttagtacaaatgtagaaagtagtatgcttcttaatcttcttgtacttggagcaggaccagatatatttgaaagttgttgttatccagtaagaaaacttatagatccagatgtagtatatgatccaagtaattatggatttggaagtataaatatagtaacatttagtccagaatatgaatatacatttaatgatataagtggaggacataatagtagtacagaaagttttatagcagatccagcaataagtcttgcacatgaacttatacatgcacttcatggactttatggagcaagaggagtaacatatgaagaaacaatagaagtaaaacaagcaccacttatgatagcagaaaaaccaataagacttgaagaatttcttacatttggaggacaagatcttaatataataacaagtgcaatgaaagaaaaaatatataataatcttcttgcaaattatgaaaaaatagcaacaagacttagtgaagtaaatagtgcaccaccagaatatgatataaatgaatataaagattattttcaatggaaatatggacttgataaaaatgcagatggaagttatacagtaaatgaaaataaatttaatgaaatatataaaaaactttatagttttacagaaagtgatcttgcaaataaatttaaagtaaaatgtagaaatacatattttataaaatatgaatttcttaaagtaccaaatcttcttgatgatgatatatatacagtaagtgaaggatttaatataggaaatcttgcagtaaataatagaggacaaagtataaaacttaatccaaaaataatagatagtataccagataaaggacttgtagaaaaaatagtaaaattttgtaaaagtgtaataccaagaaaa

SEQ ID NO: 40 - DNA, 2514 bp, Artificial Sequence
  nucleic acid sequence of HC
ggaacaaaagcaccaccaagactttgtataagagtaaataatagtgaacttttttttgtagcaagtgaaagtagttataatgaaaatgatataaatacaccaaaagaaatagatgatacaacaaatcttaataataattatagaaataatcttgatgaagtaatacttgattataatagtcaaacaataccacaaataagtaatagaacacttaatacacttgtacaagataatagttatgtaccaagatatgatagtaatggaacaagtgaaatagaagaatatgatgtagtagattttaatgtatttttttatcttcatgcacaaaaagtaccagaaggagaaacaaatataagtcttacaagtagtatagatacagcacttcttgaagaaagtaaagatatattttttagtagtgaatttatagatacaataaataaaccagtaaatgcagcactttttatagattggataagtaaagtaataagagattttacaacagaagcaacacaaaaaagtacagtagataaaatagcagatataagtcttatagtaccatatgtaggacttgcacttaatataataatagaagcagaaaaaggaaattttgaagaagcatttgaacttcttggagtaggaatacttcttgaatttgtaccagaacttacaataccagtaatacttgtatttacaataaaaagttatatagatagttatgaaaataaaaataaagcaataaaagcaataaataatagtcttatagaaagagaagcaaaatggaaagaaatatatagttggatagtaagtaattggcttacaagaataaatacacaatttaataaaagaaaagaacaaatgtatcaagcacttcaaaatcaagtagatgcaataaaaacagcaatagaatataaatataataattatacaagtgatgaaaaaaatagacttgaaagtgaatataatataaataatatagaagaagaacttaataaaaaagtaagtcttgcaatgaaaaatatagaaagatttatgacagaaagtagtataagttatcttatgaaacttataaatgaagcaaaagtaggaaaacttaaaaaatatgataatcatgtaaaaagtgatcttcttaattatatacttgatcatagaagtatacttggagaacaaacaaatgaacttagtgatcttgtaacaagtacacttaatagtagtataccatttgaacttagtagttatacaaatgataaaatacttataatatattttaatagactttataaaaaaataaaagatagtagtatacttgatatgagatatgaaaataataaatttatagatataagtggatatggaagtaatataagtataaatggaaatgtatatatatatagtacaaatagaaatcaatttggaatatataatagtagacttagtgaagtaaatatagcacaaaataatgatataatatataatagtagatatcaaaattttagtataagtttttgggtaagaataccaaaacattataaaccaatgaatcataatagagaatatacaataataaattgtatgggaaataataatagtggatggaaaataagtcttagaacagtaagagattgtgaaataatatggacacttcaagatacaagtggaaataaagaaaatcttatatttagatatgaagaacttaatagaataagtaattatataaataaatggatatttgtaacaataacaaataatagacttggaaatagtagaatatatataaatggaaatcttatagtagaaaaaagtataagtaatcttggagatatacatgtaagtgataatatactttttaaaatagtaggatgtgatgatgaaacatatgtaggaataagatattttaaagtatttaatacagaacttgataaaacagaaatagaaacactttatagtaatgaaccagatccaagtatacttaaaaattattggggaaattatcttctttataataaaaaatattatctttttaatcttcttagaaaagataaatatataacacttaatagtggaatacttaatataaatcaacaaagaggagtaacagaaggaagtgtatttcttaattataaactttatgaaggagtagaagtaataataagaaaaaatggaccaatagatataagtaatacagataattttgtaagaaaaaatgatcttgcatatataaatgtagtagatagaggagtagaatatagactttatgcagatacaaaaagtgaaaaagaaaaaataataagaacaagtaatcttaatgatagtcttggacaaataatagtaatggatagtataggaaataattgtacaatgaattttcaaaataataatggaagtaatataggacttcttggatttcatagtaataatcttgtagcaagtagttggtattataataatataagaagaaatacaagtagtaatggatgtttttggagtagtataagtaaagaaaatggatggaaagaa

SEQ ID NO: 41 - DNA, 1323 bp, Artificial Sequence
  nucleic acid sequence of LC
ccagtaaatataaaannntttaattataatgatccaataaataatgatgatataataatgatggaaccatttaatgatccaggaccaggaacatattataaagcatttagaataatagatagaatatggatagtaccagaaagatttacatatggatttcaaccagatcaatttaatgcaagtacaggagtatttagtaaagatgtatatgaatattatgatccaacatatcttaaaacagatgcagaaaaagataaatttcttaaaacaatgataaaactttttaatagaataaatagtaaaccaagtggacaaagacttcttgatatgatagtagatgcaataccatatcttggaaatgcaagtacaccaccagataaatttgcagcaaatgtagcaaatgtaagtataaataaaaaaataatacaaccaggagcagaagatcaaataaaaggacttatgacaaatcttataatatttggaccaggaccagtacttagtgataattttacagatagtatgataatgaatggacatagtccaataagtgaaggatttggagcaagaatgatgataagattttgtccaagttgtcttaatgtatttaataatgtacaagaaaataaagatacaagtatatttagtagaagagcatattttgcagatccagcacttacacttatgcatgaacttatacatgtacttcatggactttatggaataaaaataagtaatcttccaataacaccaaatacaaaagaattttttatgcaacatagtgatccagtacaagcagaagaactttatacatttggaggacatgatccaagtgtaataagtccaagtacagatatgaatatatataataaagcacttcaaaattttcaagatatagcaaatagacttaatatagtaagtagtgcacaaggaagtggaatagatataagtctttataaacaaatatataaaaataaatatgattttgtagaagatccaaatggaaaatatagtgtagataaagataaatttgataaactttataaagcacttatgtttggatttacagaaacaaatcttgcaggagaatatggaataaaaacaagatatagttattttagtgaatatcttccaccaataaaaacagaaaaacttcttgataatacaatatatacacaaaatgaaggatttaatatagcaagtaaaaatcttaaaacagaatttaatggacaaaataaagcagtaaataaagaagcatatgaagaaataagtcttgaacatcttgtaatatatagaatagcaatgtgtaaaccagtaatgtataaa

SEQ ID NO: 42 - DNA, 2565 bp, Artificial Sequence
  nucleic acid sequence of HC
aatacaggaaaaagtgaacaatgtataatagtaaataatgaagatcttttttttatagcaaataaagatagttttagtaaagatcttgcaaaagcagaaacaatagcatataatacacaaaataatacaatagaaaataattttagtatagatcaacttatacttgataatgatcttagtagtggaatagatcttccaaatgaaaatacagaaccatttacaaattttgatgatatagatataccagtatatataaaacaaagtgcacttaaaaaaatatttgtagatggagatagtctttttgaatatcttcatgcacaaacatttccaagtaatatagaaaatcttcaacttacaaatagtcttaatgatgcacttagaaataataataaagtatatacattttttagtacaaatcttgtagaaaaagcaaatacagtagtaggagcaagtctttttgtaaattgggtaaaaggagtaatagatgattttacaagtgaaagtacacaaaaaagtacaatagataaagtaagtgatgtaagtataataataccatatataggaccagcacttaatgtaggaaatgaaacagcaaaagaaaattttaaaaatgcatttgaaataggaggagcagcaatacttatggaatttataccagaacttatagtaccaatagtaggattttttacacttgaaagttatgtaggaaataaaggacatataataatgacaataagtaatgcacttaaaaaaagagatcaaaaatggacagatatgtatggacttatagtaagtcaatggcttagtacagtaaatacacaattttatacaataaaagaaagaatgtataatgcacttaataatcaaagtcaagcaatagaaaaaataatagaagatcaatataatagatatagtgaagaagataaaatgaatataaatatagattttaatgatatagattttaaacttaatcaaagtataaatcttgcaataaataatatagatgattttataaatcaatgtagtataagttatcttatgaatagaatgataccacttgcagtaaaaaaacttaaagattttgatgataatcttaaaagagatcttcttgaatatatagatacaaatgaactttatcttcttgatgaagtaaatatacttaaaagtaaagtaaatagacatcttaaagatagtataccatttgatcttagtctttatacaaaagatacaatacttatacaagtatttaataattatataagtaatataagtagtaatgcaatacttagtcttagttatagaggaggaagacttatagatagtagtggatatggagcaacaatgaatgtaggaagtgatgtaatatttaatgatataggaaatggacaatttaaacttaataatagtgaaaatagtaatataacagcacatcaaagtaaatttgtagtatatgatagtatgtttgataattttagtataaatttttgggtaagaacaccaaaatataataataatgatatacaaacatatcttcaaaatgaatatacaataataagttgtataaaaaatgatagtggatggaaagtaagtataaaaggaaatagaataatatggacacttatagatgtaaatgcaaaaagtaaaagtatattttttgaatatagtataaaagataatataagtgattatataaataaatggtttagtataacaataacaaatgatagacttggaaatgcaaatatatatataaatggaagtcttaaaaaaagtgaaaaaatacttaatcttgatagaataaatagtagtaatgatatagattttaaacttataaattgtacagatacaacaaaatttgtatggataaaagattttaatatatttggaagagaacttaatgcaacagaagtaagtagtctttattggatacaaagtagtacaaatacacttaaagatttttggggaaatccacttagatatgatacacaatattatctttttaatcaaggaatgcaaaatatatatataaaatattttagtaaagcaagtatgggagaaacagcaccaagaacaaattttaataatgcagcaataaattatcaaaatctttatcttggacttagatttataataaaaaaagcaagtaatagtagaaatataaataatgataatatagtaagagaaggagattatatatatcttaatatagataatataagtgatgaaagttatagagtatatgtacttgtaaatagtaaagaaatacaaacacaactttttcttgcaccaataaatgatgatccaacattttatgatgtacttcaaataaaaaaatattatgaaaaaacaacatataattgtcaaatactttgtgaaaaagatacaaaaacatttggactttttggaataggaaaatttgtaaaagattatggatatgtatgggatacatatgataattatttttgtataagtcaatggtatcttagaagaataagtgaaaatataaataaacttagacttggatgtaattggcaatttataccagtagatgaaggatggacagaa

SEQ ID NO: 43 - protein, 4 aa, Artificial Sequence
  potential sites of N-glycosylation on the surface of Botulinum Toxin Type A
NISE

SEQ ID NO: 44 - protein, 4 aa, Artificial Sequence
  potential sites of N-glycosylation on the surface of Botulinum Toxin Type E
NLSG

SEQ ID NO: 45 - protein, 4 aa, Artificial Sequence
  potential sites of N-glycosylation on the surface of Botulinum Toxin Type E
NGSG

SEQ ID NO: 46 - protein, 4 aa, Artificial Sequence
  potential sites of N-glycosylation on the surface of Botulinum Toxin Type E
NSSN

SEQ ID NO: 47 - protein, 4 aa, Artificial Sequence
  potential sites of N-glycosylation on the surface of Botulinum Toxin Type E
NISL

SEQ ID NO: 48 - protein, 4 aa, Artificial Sequence
  potential sites of N-glycosylation on the surface of Botulinum Toxin Type E
NDSI